(19) United States
(12) Patent Application Publication
(10) Pub. No.: US 2025/0099242 A1
(43) Pub. Date: Mar. 27, 2025

(54) LEAFLET PERFORATION TOOLS AND ASSOCIATED METHODS

(71) Applicant: EDWARDS LIFESCIENCES CORPORATION, Irvine, CA (US)

(72) Inventors: Eitan Atias, Netanya (IL); Noa Axelrod Manela, Kadima (IL)

(73) Assignee: EDWARDS LIFESCIENCES CORPORATION, Irvine, CA (US)

(21) Appl. No.: 18/971,408

(22) Filed: Dec. 6, 2024

Related U.S. Application Data
(63) Continuation of application No. PCT/US2023/026269, filed on Jun. 26, 2023.
(60) Provisional application No. 63/447,453, filed on Feb. 22, 2023.
(60) Provisional application No. 63/355,739, filed on Jun. 27, 2022.

(51) Int. Cl. A61F 2/24; A61M 25/09

(52) U.S. Cl. CPC A61F 2/2433; A61M 25/09

(57) ABSTRACT

Leaflet perforation tools and associated methods. A leaflet perforation tool can comprise a catheter, a lacerating member configured to pierce a leaflet to form a pilot puncture, and an expansion member configured to expand the pilot puncture into a leaflet opening. In another example, a leaflet perforation tool can comprise a catheter and a nosecone that comprises at least one blade. The nosecone can form a leaflet opening in a leaflet, and blade can form a leaflet slit that is connected to the leaflet opening. A method can comprise forming a leaflet opening within a leaflet with a leaflet perforation tool, positioning a replacement prosthetic valve within the leaflet opening, and radially expanding the replacement prosthetic valve. A method also can comprise forming a pilot puncture within a leaflet and expanding the pilot puncture into a leaflet opening.

### CROSS-REFERENCE TO RELATED APPLICATIONS

This application is a continuation of International Application No. PCT/US2023/026269, filed Jun. 26, 2023, which claims the benefit of U.S. Provisional Application No. 63/355,739, filed Jun. 27, 2022, and U.S. Provisional Application No. 63/447,453, filed Feb. 22, 2023, the contents of which are herein incorporated by reference in their entirety.

### FIELD

The present disclosure relates to prosthetic heart valves, and to methods and devices for modifying existing valvular structures (for example, leaflets or commissures of a native heart valve or previously-implanted prosthetic valve) prior to or during implantation of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

As surgical approaches for valve replacement become available for younger patients, patient lifetime may exceed the corresponding lifetime of the implanted prosthetic valve. Valve-in-valve (ViV) procedures have been developed to mount a new prosthetic valve within the previously-implanted prosthetic valve. However, such procedures may pose a risk of coronary artery obstruction. In particular, the leaflets of the previously-implanted prosthetic valve may block access to the coronary ostia if subsequent intervention is require or may inhibit blood flow through the frame of the new prosthetic valve to the coronary ostia. A similar problem may occur when a prosthetic valve is percutaneously expanded within a native aortic valve. In some instances, the native aortic valve may be abnormal, for example, a bicuspid aortic valve (BAV) having two leaflets. The BAV leaflets may be stiffer than normal and/or define a non-circular geometry, which may compromise the ability for a prosthetic valve having a cylindrical geometry to be successfully implanted therein. For example, a patient having a prosthetic heart valve implanted within the non-circular geometry of an unmodified BAV may be at an increased risk for annular rupture and/or reduced hemodynamic performance.

### SUMMARY

Existing methods, which rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

Described herein are examples of tools, assemblies and methods for implanting prosthetic valves and modifying leaflets of an existing valvular structure in a patient's heart.

According to one aspect of the disclosure, a leaflet perforation tool comprises a catheter comprising a lumen, and a lacerating member disposed within the lumen of the catheter. The lacerating member is configured to pierce a leaflet of a host valvular structure to form a pilot puncture in the leaflet.

In some aspects, the catheter comprises a distal end portion configured to be inserted into a patient's vasculature.

In some aspects, the leaflet perforation tool further comprises an expansion member supported by the distal end portion of the catheter.

In some aspects, the expansion member is configured to be inserted within the pilot puncture and to be selectively transitioned between a radially compressed configuration and a radially expanded configuration.

In some aspects, when the expansion member is received within the pilot puncture, transitioning the expansion member from the radially compressed configuration to the radially expanded configuration is configured to expand the pilot puncture to form a leaflet opening.

In some aspects, the lacerating member comprises a distal end portion that is configured to be selectively translated in a proximal direction and in a distal direction relative to the distal end portion of the catheter.

In some aspects, the lacerating member comprises a needle.

In some aspects, the lacerating member comprises a lumen that is configured to receive a perforation tool guidewire of the leaflet perforation tool.

In some aspects, the lacerating member comprises a guidewire.

In some aspects, the leaflet perforation tool comprises an RF energy source coupled to the guidewire that provides RF energy to a tip of the guidewire.

In some aspects, the expansion member extends at least substantially around a circumference of the catheter.

In some aspects, the expansion member comprises an inflatable balloon that is configured to be selectively inflated and deflated to transition the expansion member between the radially compressed configuration and the radially expanded configuration.

In some aspects, the expansion member comprises an expandable frame comprising a collapsible support structure that is radially expandable and compressible to transition the expandable frame between the radially compressed configuration and the radially expanded configuration.

In some aspects, the collapsible support structure comprises a plurality of frame members arranged and connected such that the collapsible support structure expands radially as the collapsible support structure is compressed axially.

In some aspects, the expandable frame comprises a distal end portion and a proximal end portion that are operatively coupled to one another via a screw mechanism, wherein rotation of the screw mechanism in a first direction causes a distance between the distal end portion and the proximal end portion to decrease and produce radial expansion of the expandable frame, and wherein rotation of the screw mechanism is a second direction causes a distance between the distal end portion and the proximal end portion to increase and produce radial compression of the expandable frame.

In some aspects, the leaflet perforation tool further comprises one or more positioning arms supported by the catheter and configured to facilitate positioning the catheter relative to the host valvular structure.

According to one aspect of the disclosure, a method of implanting a replacement prosthetic valve within a host valvular structure comprises forming, with a leaflet perforation tool, a leaflet opening within a leaflet of the host valvular structure.

In some aspects, the method further comprises positioning a replacement prosthetic valve in a radially compressed configuration within the leaflet opening.

In some aspects, the method further comprises radially expanding the replacement prosthetic valve.

In some aspects, the forming the leaflet opening comprises forming, with the leaflet perforation tool, a pilot puncture within the leaflet.

In some aspects, the forming the leaflet opening further comprises inserting a portion of the leaflet perforation tool into the pilot puncture, and expanding the pilot puncture to form the leaflet opening.

In some aspects, the leaflet perforation tool comprises a catheter and a lacerating member disposed within a lumen of the catheter, and the forming the pilot puncture within the leaflet comprises translating the lacerating member in a distal direction relative to the catheter to pierce the leaflet to form the pilot puncture.

In some aspects, the leaflet perforation tool comprises a catheter and an expansion member supported by the catheter, and the forming the leaflet opening comprises forming, with the leaflet perforation tool, a pilot puncture within the leaflet, positioning the expansion member within the pilot puncture, and transitioning the expansion member from a radially compressed configuration to a radially expanded configuration to expand the pilot puncture into the leaflet opening.

In some aspects, the leaflet perforation tool comprises a lacerating member disposed within a lumen of the catheter, and the forming the pilot puncture within the leaflet comprises translating the lacerating member in a distal direction relative to the catheter to pierce the leaflet to form the pilot puncture.

In some aspects, the method further comprises, subsequent to the forming the pilot puncture and prior to the transitioning the expansion member from the radially compressed configuration to the radially expanded configuration, retracting the lacerating member away from the leaflet.

In some aspects, the expansion member comprises an inflatable balloon, and the transitioning the expansion member from the radially compressed configuration to the radially expanded configuration comprises inflating the inflatable balloon to transition the inflatable balloon from a deflated state to an inflated state.

In some aspects, the positioning the replacement prosthetic valve within the leaflet opening comprises advancing the replacement prosthetic valve into the leaflet opening via a replacement valve guidewire.

In some aspects, the positioning the leaflet perforation tool relative to the leaflet comprises advancing the leaflet perforation tool toward the leaflet via the replacement valve guidewire.

In some aspects, the positioning the leaflet perforation tool relative to the leaflet comprises advancing the leaflet perforation tool toward the leaflet via a perforation tool guidewire that extends alongside the replacement valve guidewire.

In some aspects, the radially expanding the replacement prosthetic valve comprises inflating a valve delivery inflatable balloon.

In some aspects, the leaflet is a first leaflet, wherein the leaflet opening is a first leaflet opening formed in the first leaflet, and the method further comprises forming a second leaflet opening within a second leaflet of the host valvular structure.

According to some aspects of the disclosure, a leaflet perforation tool comprises a catheter comprising a distal end portion configured to be inserted into a patient's vasculature, a lacerating member, and an expansion member supported by the distal end portion of the catheter. The catheter comprises a lumen, and the lacerating member is disposed within the lumen of the catheter. The lacerating member is configured to pierce a leaflet of a host valvular structure to form a pilot puncture in the leaflet. The expansion member is configured to be inserted within the pilot puncture and to be selectively transitioned between a radially compressed configuration and a radially expanded configuration. When the expansion member is received within the pilot puncture, transitioning the expansion member from the radially compressed configuration to the radially expanded configuration expands the pilot puncture to form a leaflet opening.

According to some aspects of the disclosure, a leaflet perforation tool comprises a catheter comprising a distal end portion configured to be inserted into a patient's vasculature and a nosecone coupled to the distal end portion of the catheter. The nosecone comprises a main body with an external surface and at least one blade coupled to the main body. The nosecone is configured to be inserted through and form a leaflet opening in a leaflet of a host valvular structure.

According to some aspects of the disclosure, a leaflet perforation tool comprises a catheter comprising a distal end portion configured to be inserted into a patient's vasculature and a nosecone coupled to the distal end portion of the catheter. The nosecone comprises a main body with an external surface and at least one blade recessed within the main body. The leaflet perforation tool is configured such that, when the nosecone is inserted within a leaflet of a host valvular structure, the nosecone forms a leaflet opening within the leaflet and the blade forms a leaflet slit that is connected to the leaflet opening.

According to some aspects of the disclosure, a method of implanting a replacement prosthetic valve within a host valvular structure comprises forming, with a leaflet perforation tool, a leaflet opening within a leaflet of a host valvular structure. The method further comprises positioning a replacement prosthetic valve in a radially compressed configuration within the leaflet opening and radially expanding the replacement prosthetic valve.

According to some aspects of the disclosure, a method comprises forming a pilot puncture within a leaflet of a host valvular structure with a laceration member of a leaflet perforation tool. The method further comprises positioning an expansion member of the leaflet perforation tool within the pilot puncture and transitioning the expansion member from a radially compressed configuration to a radially expanded configuration to expand the pilot puncture into the leaflet opening.

According to some aspects of the disclosure, a method comprises forming, with a leaflet perforation tool, a tear or opening within a leaflet of a host valvular structure. The leaflet perforation tool comprises a catheter and a nosecone coupled to a distal end of the catheter, and the nosecone comprises a main body and at least one blade coupled to the main body.

According to some aspects of the disclosure, there is provided a delivery assembly, comprising a delivery apparatus and a prosthetic valve. The delivery apparatus comprises a handle, a first balloon, a second balloon, a balloon catheter extending from the handle, and a perforating member. The first balloon is configured to transition between deflated and inflated states thereof. The second balloon is configured to transition between deflated and inflated states thereof. The balloon catheter defines a balloon catheter lumen. The balloon catheter is in fluid communication with at least one of the first balloon and the second balloon. The perforating member is configured to pierce a host leaflet of a host valvular structure to form a pilot puncture in the host leaflet. The first balloon is configured to be inserted within the pilot puncture.

According to some aspects of the disclosure, there is provided a method of implanting a prosthetic valve within a host valvular structure, the method comprising advancing a delivery assembly that includes a delivery apparatus carrying a prosthetic valve in a radially compressed state, to a host valvular structure. The delivery apparatus further comprises a perforating member, a first balloon, and a second balloon. The method further comprises forming, with the perforating member and the first balloon, a leaflet opening within a host leaflet of the host valvular structure. The method further comprises inflating the second balloon, while the prosthetic valve is disposed therearound, inside the host valvular structure, so as to radially expand the prosthetic valve.

According to some aspects of the disclosure, there is provided a delivery assembly comprising a guest prosthetic valve and a delivery apparatus. The guest prosthetic comprises a frame movable between a radially compressed and a radially expanded configuration. The delivery apparatus comprises a handle, a balloon catheter extending from the handle, a valve expansion balloon mounted on the balloon catheter, a hole dilation balloon positioned distal to the valve expansion balloon, and a perforating member. The balloon catheter defines a balloon catheter lumen. The valve expansion is in fluid communication with the balloon catheter lumen, and is configured to transition between deflated and inflated states thereof. The hole dilation balloon is in fluid communication with the valve expansion balloon, and is configured to transition between deflated and inflated states thereof. The perforating member is configured to pierce a host leaflet of a host valvular structure to form a pilot puncture in the host leaflet, when a distal end portion of the perforating member is positioned distal to the hole dilation balloon. The maximum diameter of the valve expansion balloon in its inflated state is greater than the maximum diameter of the hole dilation balloon in its inflated state. The hole dilation balloon is configured to be inserted within the pilot puncture. Inflation of the hole dilation balloon, when positioned within the pilot puncture, is configured to expand the pilot puncture to form a leaflet opening. When the guest prosthetic valve is disposed around the valve expansion balloon and positioned within the host valvular structure, inflation of the valve expansion balloon expands the guest prosthetic valve to implant the guest prosthetic valve in the host valvular structure.

According to some aspects of the disclosure, there is provided a delivery assembly, comprising a guest prosthetic valve and a delivery apparatus. The guest prosthetic valve comprises a frame movable between a radially compressed and a radially expanded configuration. The delivery apparatus comprises a handle, a balloon catheter extending from the handle, a nosecone shaft extending through the balloon catheter lumen, a nosecone attached to a distal end of the nosecone shaft, a valve expansion balloon mounted on the balloon catheter, a hole dilation balloon positioned distal to the valve expansion balloon, and a perforating member. The balloon catheter defines a balloon catheter lumen. The nosecone shaft defines a nosecone shaft lumen. The valve expansion balloon is in fluid communication with the balloon catheter lumen, and is configured to transition between deflated and inflated states thereof. The hole dilation balloon is in fluid communication with the nosecone shaft lumen, and is configured to transition between deflated and inflated states thereof. The perforating member is configured to pierce a host leaflet of a host valvular structure to form a pilot puncture in the host leaflet, when a distal end portion of the perforating member is positioned distal to the hole dilation balloon. The maximum diameter of the valve expansion balloon in its inflated state is greater than the maximum diameter of the hole dilation balloon in its inflated state. The valve expansion balloon and the hole dilation balloon are fluidly separated from each other. The hole dilation balloon is configured to be inserted within the pilot puncture. Inflation of the hole dilation balloon, when positioned within the pilot puncture, is configured to expand the pilot puncture to form a leaflet opening. When the guest prosthetic valve is disposed around the valve expansion balloon and positioned within the host valvular structure, inflation of the valve expansion balloon expands the guest prosthetic valve to implant the guest prosthetic valve in the host valvular structure.

According to some aspects of the disclosure, there is provided a delivery assembly, comprising a guest prosthetic valve and a delivery apparatus. The guest prosthetic valve comprises a frame movable between a radially compressed and a radially expanded configuration. The delivery apparatus comprises a handle, a balloon catheter extending from the handle, an inner balloon mounted on the balloon catheter, an outer balloon disposed around the inner balloon, and a perforating member. The balloon catheter defines a balloon catheter lumen, and is in fluid communication with the balloon catheter lumen. The inner balloon comprises an inner balloon wall, wherein the inner balloon is configured to transition between deflated and inflated states thereof. The outer balloon comprises an outer balloon wall, wherein the outer balloon is configured to transition between deflated and inflated states thereof. The perforating member is configured to pierce a host leaflet of a host valvular structure to form a pilot puncture in the host leaflet, when a distal end portion of the perforating member is positioned distal to the inner. The maximum diameter of the outer balloon in its inflated state is greater than the maximum diameter of the inner balloon in its inflated state. The inner balloon is configured to transition from a sealed configuration to an unsealed configuration. The inner and outer balloons are configured to be inserted within the pilot puncture. Inflation of the inner balloon, when the inner and outer balloons are positioned within the pilot puncture, is configured to expand the pilot puncture to form a leaflet opening. When the guest prosthetic valve is disposed around the outer balloon and positioned within the host valvular structure, inflation of the outer balloon expands the guest prosthetic valve to implant the guest prosthetic valve in the host valvular structure.

According to some aspects of the disclosure, there is provided a delivery assembly, comprising a guest prosthetic valve and a delivery apparatus. The guest prosthetic valve comprises a frame movable between a radially compressed and a radially expanded configuration. The delivery apparatus comprises a handle, a balloon catheter extending from the handle, a nosecone shaft extending through the balloon catheter lumen, a nosecone attached to a distal end of the nosecone shaft, an outer balloon mounted on the balloon catheter, an inner balloon disposed inside the outer balloon, and a perforating member. The balloon catheter defines a balloon catheter lumen. The nosecone shaft defines a nosecone shaft lumen. The outer balloon is in fluid communication with the balloon catheter lumen, and is configured to transition between deflated and inflated states thereof. The inner balloon is in fluid communication with the nosecone shaft lumen, and is configured to transition between deflated and inflated states thereof. The perforating member is configured to pierce a host leaflet of a host valvular structure to form a pilot puncture in the host leaflet, when a distal end portion of the perforating member is positioned distal to the inner balloon. The maximum diameter of the outer balloon in its inflated state is greater than the maximum diameter of the inner balloon in its inflated state. The inner balloon and the outer balloon are fluidly separated from each other. The inner and outer balloons are configured to be inserted within the pilot puncture. Inflation of the inner balloon via the nosecone shaft lumen, when the inner and outer balloons are positioned within the pilot puncture, is configured to expand the pilot puncture to form a leaflet opening. When the guest prosthetic valve is disposed around the outer balloon and positioned within the host valvular structure, inflation of the outer balloon via the balloon catheter lumen expands the guest prosthetic valve to implant the guest prosthetic valve in the host valvular structure

According to some aspects of the disclosure, there is provided a method of implanting a guest prosthetic valve within a host valvular structure, the method comprising advancing a delivery assembly that comprises a delivery apparatus carrying a guest prosthetic valve in a radially compressed state, to a host valvular structure. The delivery apparatus comprises a perforating member, a hole dilation balloon, and a valve expansion balloon proximal to the hole dilation balloon. The method further comprises forming, with the perforating member, a pilot puncture within a host leaflet of the host valvular structure. The method further comprises positioning the hole dilation balloon, in a deflated state thereof, within the pilot puncture. The method further comprises inflating the hole dilation balloon to expand the pilot puncture and form a leaflet opening within the host leaflet. The hole dilation balloon is inflatable to a maximum diameter D1. The method further comprises deflating the hole dilation balloon. The method further comprises positioning the valve expansion balloon in a deflated state thereof, along with the guest prosthetic valve disposed in a compressed state over the valve expansion balloon, inside the host valvular structure. The method further comprises inflating the valve expansion balloon to a diameter that exceeds D1, so as to radially expand the guest prosthetic valve.

According to some aspects of the disclosure, there is provided a method of implanting a guest prosthetic valve within a host valvular structure, the method comprising advancing a delivery assembly that comprises a delivery apparatus carrying a guest prosthetic valve in a radially compressed state, to a host valvular structure. The delivery apparatus comprises an inner balloon, an outer balloon disposed around the inner balloon, and a perforating member. The inner balloon is in a sealed configuration, in which it is fluidly sealed from a cavity of the outer balloon, during advancing of the delivery assembly to the host valvular structure. The method further comprises forming, with the perforating member, a pilot puncture within a host leaflet of the host valvular structure. The method further comprises positioning the inner and outer balloons, in deflated states thereof, within the pilot puncture. The method further comprises inflating the inner balloon to expand the pilot puncture and form a leaflet opening within the host leaflet. The inner balloon is inflatable to a maximum diameter D1. The method further comprises inflating the outer balloon with the guest prosthetic valve disposed in a compressed state thereover, inside the host valvular structure to a diameter that exceeds D1, so as to radially expand the guest prosthetic valve.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a cross-sectional view of a native aortic valve.

FIG. 2A shows a cross-sectional view of a prosthetic valve implanted in the native aortic valve of FIG. 1, according to an example.

FIG. 2B shows the implanted prosthetic valve of FIG. 2A as viewed from the ascending aorta, according to an example.

FIG. 3 shows a valve-in-valve implantation within the native aortic valve of FIG. 1, according to an example.

FIG. 4A is a simplified side view of a leaflet perforation tool positioned on an outflow side of an existing valvular structure according to an example.

FIG. 4B is a simplified side view of the leaflet perforation tool of FIG. 4A with a needle piercing the existing valvular structure.

FIG. 4C is a simplified side view of the leaflet perforation tool of FIG. 4A with an inflatable balloon positioned within the existing valvular structure in a deflated state.

FIG. 4D is a simplified side view of the leaflet perforation tool of FIG. 4A with an inflatable balloon positioned within the existing valvular structure in an inflated state.

FIG. 4E is a simplified side view of the leaflet perforation tool of FIG. 4A with the inflatable balloon deflated within the leaflet opening.

FIG. 4F is a simplified side view of the leaflet perforation tool of FIG. 4A retracted from the valvular structure.

FIG. 4G is a simplified side view of a replacement prosthetic valve positioned in a crimped configuration inside the leaflet opening formed in the existing valvular structure.

FIG. 4H is a simplified side view of the replacement prosthetic valve of FIG. 4G expanded inside the existing valvular structure.

FIG. 5A shows a leaflet perforation tool with an inflatable balloon positioned within a valvular structure in a deflated state according to another example.

FIG. 5B shows the leaflet perforation tool of FIG. 5A with the inflatable balloon in an inflated state.

FIG. 5C shows a replacement prosthetic valve positioned in a crimped configuration thereof within a leaflet opening formed in the valvular structure of FIG. 5B.

FIG. 6A shows the valvular structure of FIGS. 5A-5B with a perforated leaflet.

FIG. 6B shows a prosthetic valve in a radially expanded configuration within the valvular structure of a previously implanted prosthetic valve according to an example.

FIG. 7A shows an expandable frame for expanding a perforation in an existing valvular structure in a radially compressed configuration according to an example.

FIG. 7B shows the expandable frame of FIG. 7A in a radially expanded configuration.

FIG. 8 is a flowchart depicting examples of methods of implanting a replacement prosthetic valve within a host valvular structure using a leaflet perforation tool that comprises an expansion member.

FIG. 9 shows a leaflet perforation tool with aligned blades according to an example.

FIG. 10 is a cross-sectional view of the leaflet perforation tool of FIG. 9 as viewed along line 10-10 in FIG. 9.

FIG. 11 is a cross-sectional view of the leaflet perforation tool of FIG. 9 positioned on an outflow side of an existing valvular structure according to an example.

FIG. 12A shows the leaflet perforation tool of FIG. 9 positioned on an outflow side of an existing valvular structure with a lacerating member forming a pilot puncture in a leaflet according to an example.

FIG. 12B shows the leaflet perforation tool of FIG. 12A with a nosecone inserted within the pilot puncture to form a leaflet opening according to an example.

FIG. 13A shows a leaflet opening and a pair of leaflet slits that may be formed by a leaflet perforation tool according to an example.

FIG. 13B shows the leaflet opening of FIG. 12A upon being further expanded by opening the leaflet slits.

FIG. 14 shows a leaflet perforation tool with offset blades according to an example.

FIG. 15 is a cross-sectional view of the leaflet perforation tool of FIG. 14 as viewed along the line 15-15 in FIG. 14.

FIG. 16A shows the leaflet perforation tool of FIG. 14 positioned on an outflow side of an existing valvular structure with a lacerating member forming a pilot puncture in a leaflet according to an example.

FIG. 16B shows the leaflet perforation tool of FIG. 16A with a nosecone inserted within the pilot puncture to form a leaflet opening according to an example.

FIG. 17 is a flowchart depicting examples of methods of implanting a replacement prosthetic valve within a valvular structure using a leaflet perforation tool that comprises one or more blades coupled to a nosecone.

FIG. 18 shows an exemplary delivery assembly comprising a delivery apparatus carrying a balloon expandable prosthetic device, wherein the delivery assembly includes a tandem balloon arrangement.

FIG. 19 shows a cross sectional view of an exemplary delivery apparatus comprising an intermediate connector disposed between a hole dilation balloon and a valve expansion balloon.

FIG. 20 shows a cross sectional view of an exemplary delivery apparatus comprising a hole dilation balloon and a valve expansion balloon attached to each other at attachment ends thereof which are spaced from an inner catheter extending through the cavities of the balloons.

FIG. 21 shows a cross sectional view of an exemplary delivery apparatus comprising a hole dilation balloon comprising a thinner wall than the wall of the valve expansion balloon.

FIG. 22 shows a cross sectional view of an exemplary delivery apparatus comprising a non-uniform unitary balloon.

FIG. 23A is a simplified side view of a delivery apparatus with a tandem balloon arrangement, positioned on an outflow side of a host leaflet according to an example.

FIG. 23B is a simplified side view of the delivery apparatus of FIG. 23A with a needle piercing the host leaflet.

FIG. 23C is a simplified side view of the delivery apparatus of FIG. 23A with a hole dilation balloon positioned within the host leaflet in a deflated state.

FIG. 23D is a simplified side view of the delivery apparatus of FIG. 23A with the hole dilation balloon positioned within the host leaflet in an inflated state.

FIG. 24A shows the delivery apparatus of FIG. 23A with the hole dilation balloon positioned within a pilot puncture of the host leaflet in a deflated state.

FIG. 24B shows the hole dilation balloon of FIG. 24A inflated within the host leaflet.

FIG. 25A is a simplified side view of the delivery apparatus of FIG. 23A with the hole dilation balloon positioned within a leaflet opening in a deflated state.

FIG. 25B is a simplified side view of the delivery apparatus of FIG. 25A with a guest prosthetic valve compressed over a deflated valve expansion balloon and positioned within the leaflet opening of the host leaflet, while a cover shaft is disposed between the guest prosthetic valve and the valve expansion balloon.

FIG. 25C is a simplified side view of the delivery apparatus of FIG. 25A with the guest prosthetic valve compressed over a deflated valve expansion balloon and positioned within the leaflet opening of the host leaflet, while a cover shaft is no longer disposed around the valve expansion balloon.

FIG. 25D is a simplified side view of the delivery apparatus of FIG. 25A with the guest prosthetic valve expanded by the inflated valve expansion balloon inside the host valvular structure.

FIG. 26A is a simplified side view of a delivery apparatus with a tandem balloon arrangement, wherein a hole dilation balloon is positioned within a leaflet opening in a deflated state, and a cover shaft is disposed around a guest prosthetic valve compressed over a deflated valve expansion balloon.

FIG. 26B is a simplified side view of the delivery apparatus of FIG. 26A with the guest prosthetic valve compressed over the deflated valve expansion balloon and covered by the cover shaft, while the prosthetic valve is positioned within the leaflet opening.

FIG. 26C is a simplified side view of the delivery apparatus of FIG. 26A with the guest prosthetic valve compressed over a deflated valve expansion balloon and positioned within the leaflet opening of the host leaflet, while a cover shaft no longer disposed around the guest prosthetic valve.

FIG. 27 shows a cross-sectional view of an exemplary delivery apparatus with a tandem balloon arrangement, comprising a hole dilation balloon and a valve expansion balloon sealed from each other.

FIG. 28 shows a perspective view of an exemplary delivery assembly comprising a delivery apparatus carrying a balloon expandable prosthetic device, wherein the delivery assembly includes a balloon-in-balloon arrangement.

FIG. 29 shows a cross sectional view of an exemplary delivery apparatus comprising a balloon-in-balloon arrangement, with the inner balloon having a thinner wall than that of the outer balloon.

FIG. 30 shows a cross sectional view of an exemplary delivery apparatus comprising a balloon-in-balloon arrangement, with the inner balloon comprising weakened portions.

FIG. 31A shows the delivery apparatus of FIG. 30 with the inner balloon inflated.

FIG. 31B shows the delivery apparatus of FIG. 30 with the inner balloon torn and the outer balloon in an inflated state.

FIG. 32A shows an exemplary inner balloon in a sealed configuration, the inner balloon wall comprising a tear-line, with a ripcord attached at a distal end thereof to the tear-line.

FIG. 32B shows the inner balloon of FIG. 32A in an unsealed configuration, with the ripcord pulled to form a tear along the tear-line.

FIG. 33A shows an exemplary inner balloon comprising a flexible port folded over itself by a loop attached to a release cord to retain the inner balloon in a sealed configuration.

FIG. 33B shows the inner balloon of FIG. 33A in an unsealed configuration, with the flexible port unfolded by removing the loop therefrom.

FIG. 34A is a simplified side view of a delivery apparatus with a balloon-in-balloon arrangement, positioned on an outflow side of a host leaflet according to an example.

FIG. 34B is a simplified side view of the delivery apparatus of FIG. 34A with a needle piercing the host leaflet.

FIG. 34C is a simplified side view of the delivery apparatus of FIG. 34A with both balloons positioned within the host leaflet, in deflated states of both balloons.

FIG. 34D is a simplified side view of the delivery apparatus of FIG. 34A with both balloons positioned within the host leaflet in an inflated state of the inner balloon and a deflated state of the outer balloon.

FIG. 34E is a simplified side view of the delivery apparatus of FIG. 34A with both balloons positioned within a leaflet opening formed in the host leaflet, in deflated states of both balloons.

FIG. 34F is a simplified side view of the delivery apparatus of FIG. 34A with both balloons positioned on an outflow side of a host leaflet, and a guest prosthetic valve disposed around the outer balloon in deflated states of both balloon.

FIG. 34G is a simplified side view of the delivery apparatus of FIG. 34A with the guest prosthetic valve compresses over the outer balloon, and positioned, along with both balloon, in deflated states thereof, inside the leaflet opening.

FIG. 34H is a simplified side view of the delivery apparatus of FIG. 34A with the guest prosthetic valve expanded by the inflated outer balloon, with the inner balloon in an unsealed configuration.

FIG. 35 shows a cross sectional view of an exemplary delivery apparatus with a balloon-in-balloon arrangement, comprising an inner balloon in fluid communication with a lumen of the nosecone shaft, and an outer balloon in fluid communication with a lumen of the balloon catheter.

FIG. 36 is a flow chart depicting examples of methods of implanting a prosthetic valve within a host valvular structure, utilizing a delivery apparatus comprising a first balloon and a second balloon.

FIG. 37 is a flow chart depicting examples of methods of implanting a prosthetic valve within a host valvular structure, utilizing a delivery apparatus comprising a hole dilation balloon and a valve expansion balloon proximal to the hole dilation balloon.

FIG. 38 is a flow chart depicting examples of methods of implanting a prosthetic valve within a host valvular structure, utilizing a delivery apparatus comprising an inner balloon and an outer balloon disposed around the inner balloon.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic valve assembly and implantation and structures of the prosthetic valve, “proximal” refers to a position, direction, or portion of a component that is closer to the user and a handle of the delivery system or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or.”

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

### Examples of the Disclosed Technology

Described herein are devices and methods for implanting prosthetic valves and modifying leaflets of an existing valvular structure in a patient's heart. Prior to or during implantation of the prosthetic valve within the existing valvular structure, each device, such as a leaflet perforation tool and/or a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some examples, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

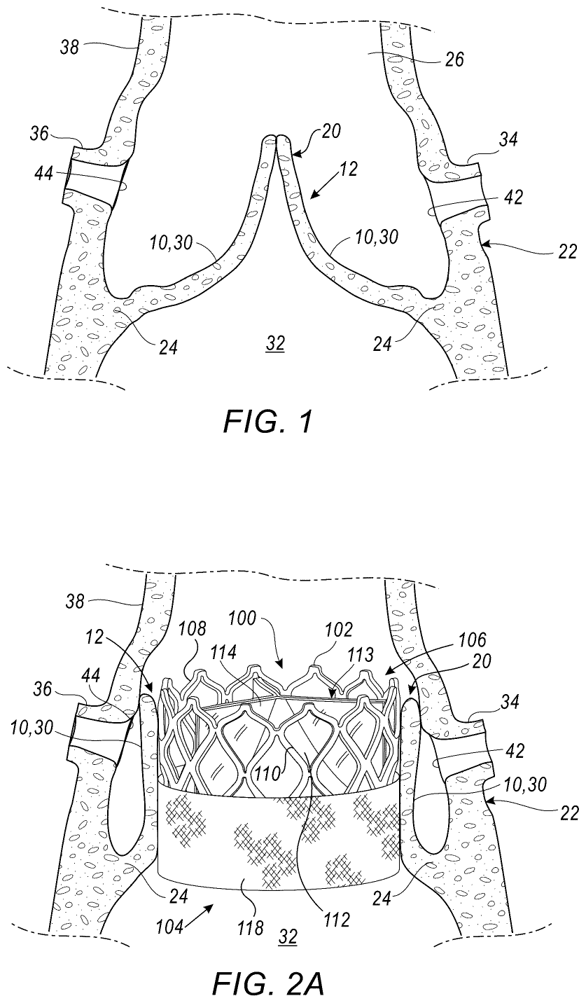

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures 40 (see FIG. 1B) by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

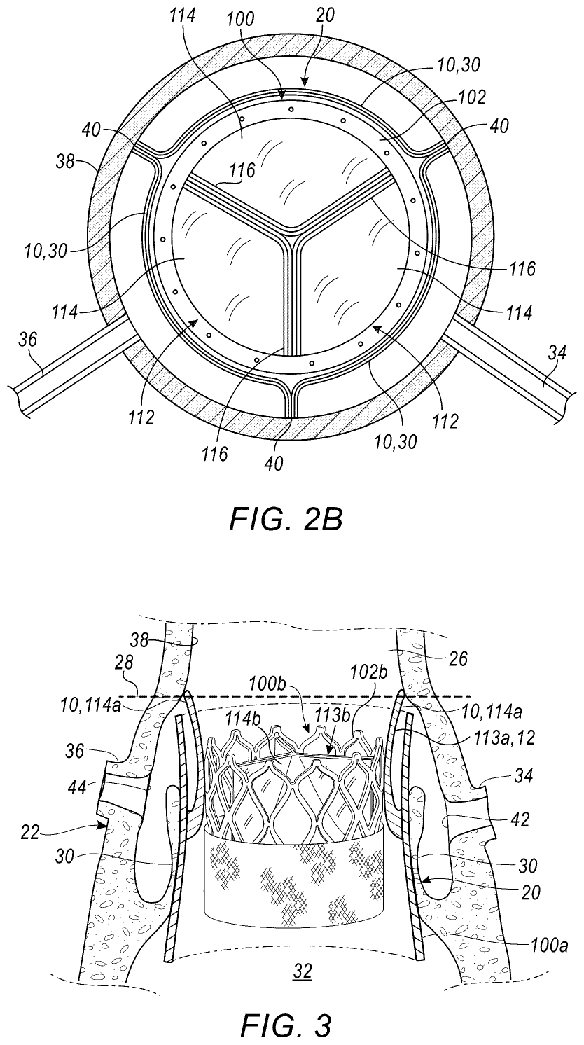

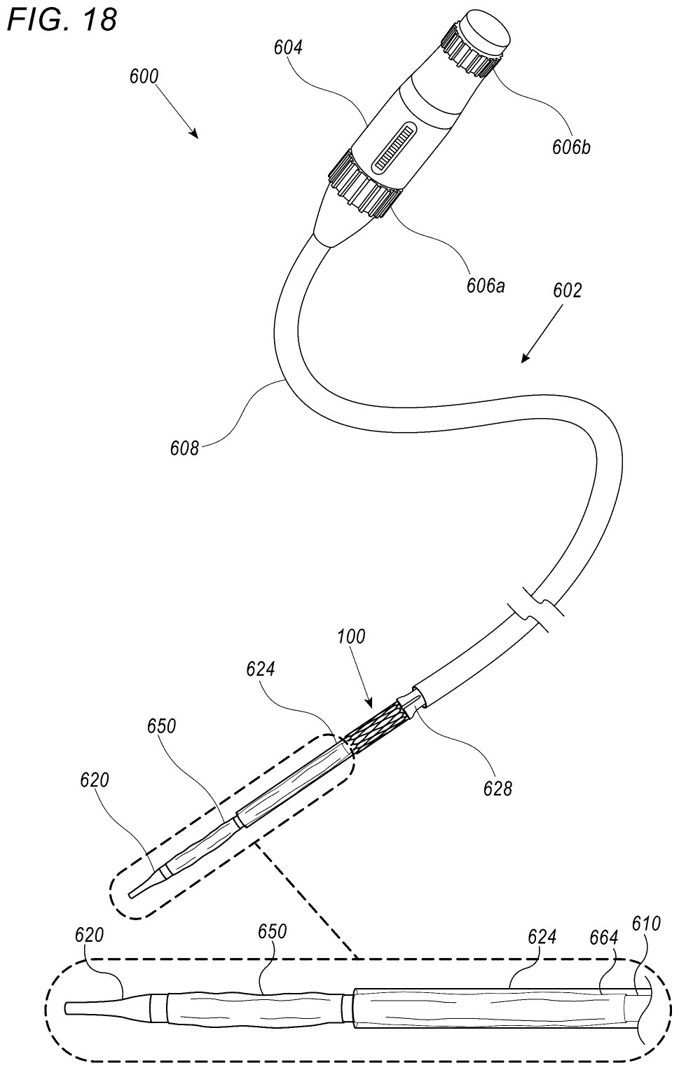

FIGS. 2A-2B show an exemplary prosthetic valve 100 that can be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (such as delivery apparatus 602 described below with respect to FIG. 18, as well as other examples of delivery apparatuses described throughout the current disclosure) in the radially compressed state during delivery, and then expanded to the radially expanded state once the prosthetic valve reaches the implantation site. The expanded state may include a range of diameters to which the valve may expand, between the compressed state and a maximum diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and the like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic valve is mounted within the existing, degrading prosthetic valve in order to restore proper function. FIG. 3 illustrates an exemplary hypothetical coronary artery obstruction that could occur in some cases from implantation of a prosthetic valve 100^{b} within a previously implanted prosthetic valve 100a (for example, after a ViV procedure). In this example, the prosthetic valve 100^{b} is the guest valve or new valve, and the prosthetic valve 100a is the host valve or old valve. In this example, the prosthetic valve 100a was previously implanted within the orifice of the native aortic valve 20. Each of the prosthetic valves 100a, 100^{b} can have the general structure of the prosthetic valve 100 described with reference to FIGS. 2A-2B, though in some examples, each of the prosthetic valves 100a, 100^{b} can be a different type of prosthetic valve. For example, a balloon expandable guest valve 100^{b} can be implanted inside a previously implanted mechanically expandable or self-expandable host valve 100a.

During implantation of the prosthetic valve 100^{b}, the prosthetic valve 100^{b} is positioned within a central region defined between the leaflets 114a of the prosthetic valve 100a, which now take the role of host leaflet 10. The prosthetic valve 100^{b} is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100^{b} can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100^{b} is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic valve (known as a “surgical valve”) or a transcatheter heart valve. The term “guest valve”, as used herein, refers to a prosthetic valve implanted in a host valve, which can be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100^{b} is implanted. It is to be understood that any reference to a “prosthetic valve” throughout the disclosure and the claims, without any specific reference to any of the terms “guest” or “host”, refers to a guest prosthetic valve that can be implanted in a native valve or in a previously implanted prosthetic valve. The terms “guest prosthetic valve” and “replacement prosthetic valve”, as used herein, are interchangeable. The terms “prosthetic valve” and “prosthetic heart valve”, as used herein, are interchangeable.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36. Additionally or alternatively, when the existing valvular structure is a BAV, the modification can allow for improved mounting of the cylindrical prosthetic valve, which would otherwise be compromised by the non-circular geometry of the native BAV.

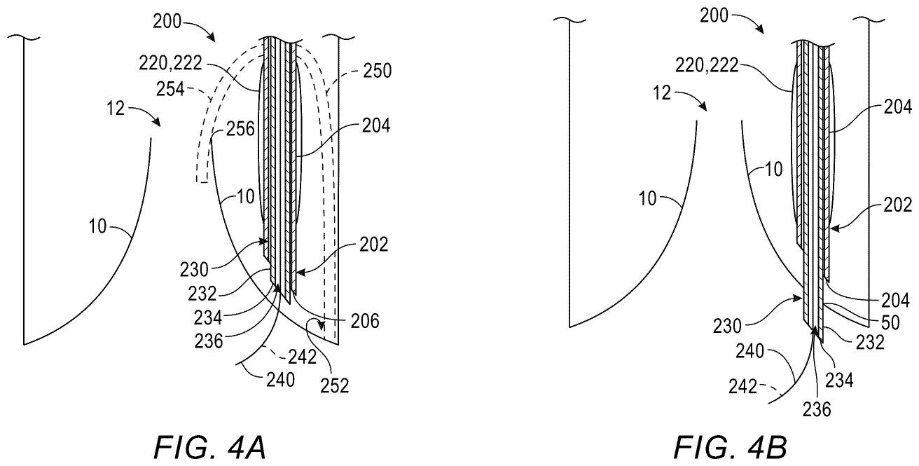

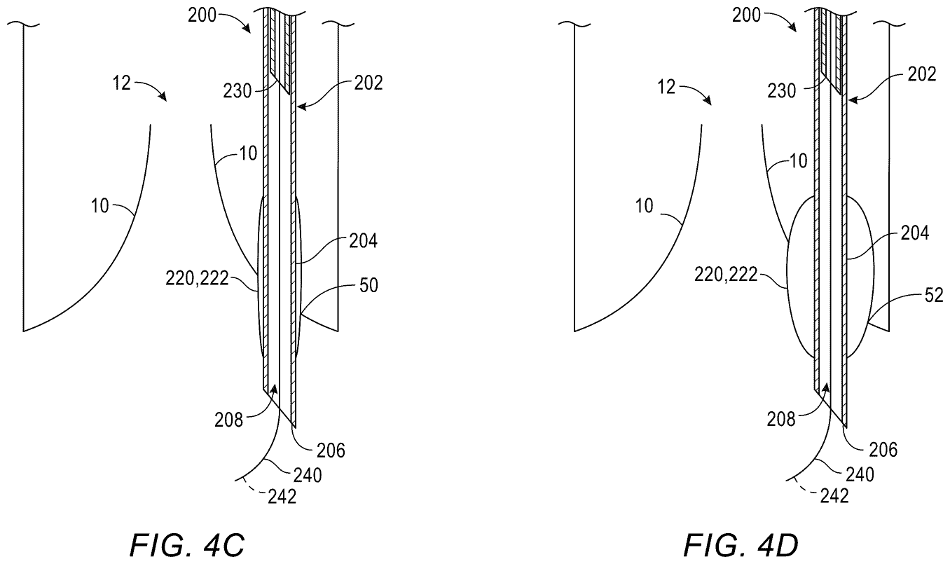

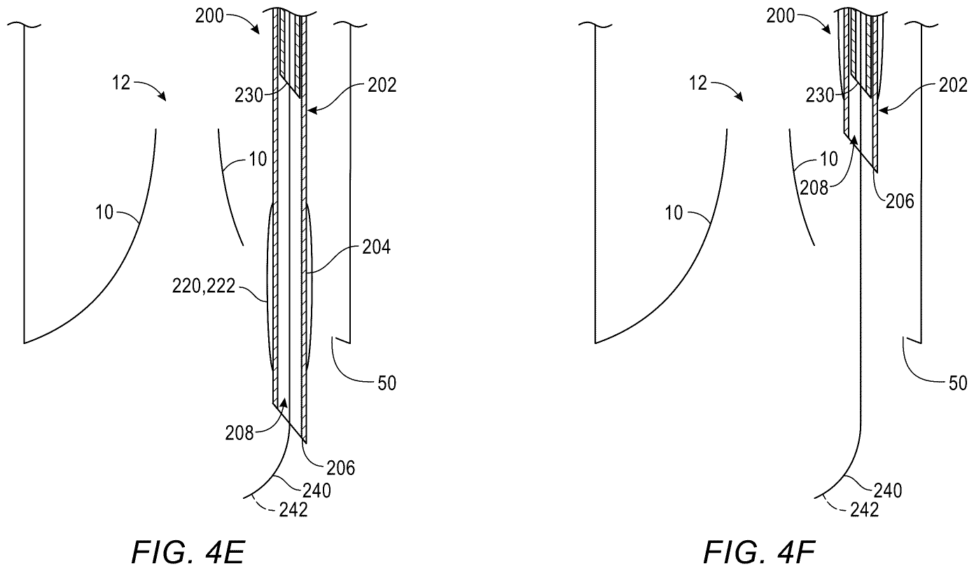

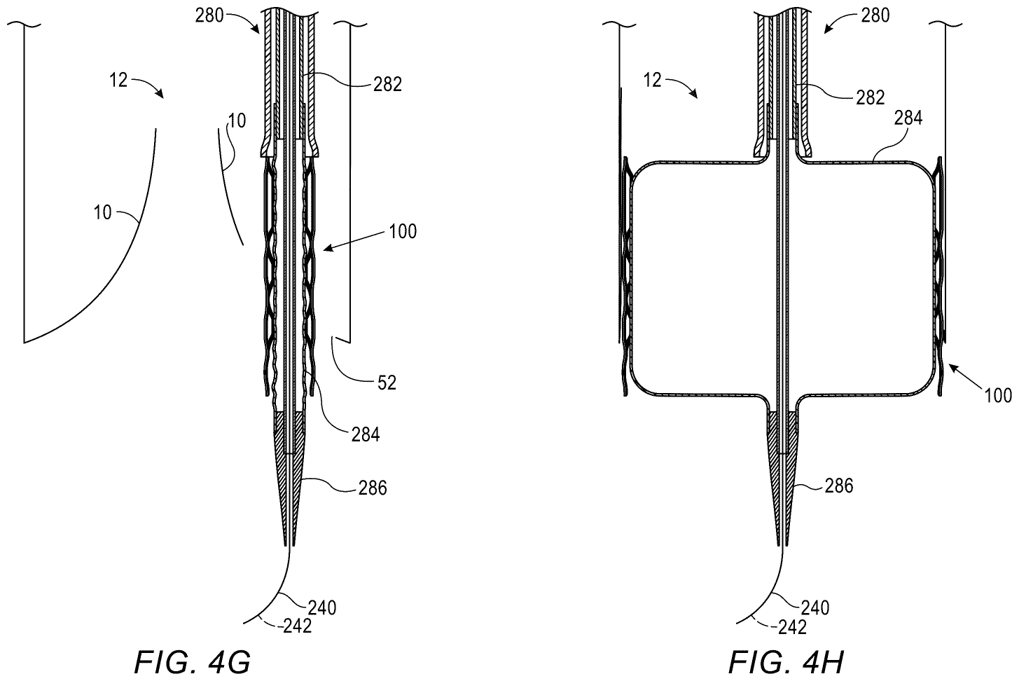

FIGS. 4A-4D illustrate a first exemplary tool 200 that can be used to perforate, cut, and/or tear a host leaflet 10 of a host valvular structure 12, such as a native leaflet 30 of a native valvular structure 29 or a prosthetic valve leaflet 114 of a prosthetic valve 100. In the present disclosure, the tool 200 also may be referred to as a leaflet perforation tool 200 and/or as a first leaflet perforation tool 200. The leaflet perforation tool 200 comprises a catheter 202 with a distal end portion 204 configured to be inserted into a patient's vasculature, such as within an ascending aorta 26. The tool 200 additionally comprises an expansion member 220 supported by the catheter 202. As described in more detail below, the expansion member 220 is configured to be selectively transitioned between a radially compressed configuration and a radially expanded configuration to expand an opening within the valvular structure.

The leaflet perforation tool 200 further comprises a lacerating member 230 disposed within a lumen 208 of the catheter 202. The lacerating member 230 comprises a distal end portion 232 that is configured to be selectively translated in the proximal or distal directions relative to the distal end portion 204 of the catheter 202. In some examples, the catheter 202 and the lacerating member 230 are configured to be movable axially relative to each other in the proximal and distal directions and/or rotationally movable relative to each other. The proximal end portions of the catheter 202 and the lacerating member 230 can be coupled to a handle (which can be similar to handle 604 illustrated in FIG. 18) of the tool 200. The handle can have one or more actuators (for example, in the form of rotatable knobs such as knob 606 illustrated in FIG. 18) that are operatively coupled to the catheter 202 and/or the lacerating member 230 to produce axial and/or rotational movement of the catheter 202 and/or the lacerating member 230.

As shown in FIG. 4B, the lacerating member 230 is configured to puncture a leaflet 10 of a host valvular structure 12 to form a pilot puncture 50 within the leaflet 10. Subsequent to forming the pilot puncture 50, and as shown in FIG. 4C, the expansion member 220 may be inserted within the pilot puncture 50. With the expansion member 220 received within the pilot puncture 50, transitioning the expansion member 220 from the radially compressed configuration (FIG. 4C) to the radially expanded configuration (FIG. 4D) can expand the pilot puncture 50 to form a leaflet opening 52 that is sized to receive the guest prosthetic valve 100 in the radially collapsed configuration. As described in more detail below, expanding the frame of a replacement prosthetic valve to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame of the replacement prosthetic valve.

In some examples, expanding the expansion member 220 to the radially expanded configuration within the leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In other examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

Additionally or alternatively, in some examples, expanding the expansion member 220 to the radially expanded configuration within the leaflet 10 may cause the leaflet 10 to rip and/or tear such that the leaflet opening 52 is not a bounded hole. Stated differently, in such examples, the leaflet opening 52 may be formed by a tear that extends from the pilot puncture 50 fully to the free edge of the leaflet 10 (the coaptation edge of the leaflet).

The leaflet perforation tool 200 may be configured to form the leaflet opening 52 in any of a variety of host valvular structures. In the example of FIGS. 4A-4D, the host valvular structure 12 can be the valvular structure of a previously implanted prosthetic valve, such as the prosthetic valve 100a of FIG. 3. In such examples, using the leaflet perforation tool 200 as described herein to form the leaflet opening 52 in a previously implanted prosthetic valve may represent a method and/or step for preparing a guest prosthetic valve 100^{b} for implanting within the previously implanted prosthetic valve 100a (for example, via a ViV procedure).

Similarly, the host valvular structure 12 in the example of FIGS. 4A-4D can be a valvular structure 29 of a native heart valve, such as the native heart valve 20 shown in FIGS. 2A-2B. In such examples, the lacerating member 230 can be configured to puncture a leaflet 30 of the native heart valve 20.

In some examples, the host valvular structure 12 may be a valvular structure 29 of a native heart valve, such as the native aortic valve 20 illustrated in FIGS. 2A-2B. In some examples, the host valvular structure and/or the native valve may refer to another valve of a patient's heart, such as a mitral valve, a pulmonary valve, or a tricuspid valve.

In some examples, the lacerating member 230 may include and/or be a needle, such as a spring-loaded needle and/or a Veress needle. As shown in FIGS. 4A-4D, the distal end portion 232 of the lacerating member 230 can terminate at an angled surface 234. The angled surface can have a sharp cutting edge to facilitate piercing the leaflet 10 when the needle is pressed against the leaflet. As further shown in FIGS. 4A-4D, the distal end portion 204 of the catheter 202 also can terminate at an angled surface 206 that is angled to be aligned with the angled surface 234 of the lacerating member 230. For example, the respective terminal ends of the catheter 202 and/or the lacerating member 230 can be shaped such that, when the distal end portion 232 of the lacerating member 230 is axially aligned with the distal end portion 204 of the catheter 202, the angled surface 206 of the catheter 202 is at least substantially coplanar with the angled surface 234 of the lacerating member 230.

The leaflet perforation tool 200 may include any of a variety of features to facilitate positioning the catheter 202 and/or the lacerating member 230 relative to the leaflet 10. For example, the catheter 202 and/or the lacerating member 230 may be pre-formed, shaped, and/or curved such that the catheter 202 and/or the lacerating member 230 may be directed and/or angled toward the leaflet 10 when positioned on an outflow side of the host valvular structure.

In some examples, and as shown in FIGS. 4A-4D, the lacerating member 230 may comprise a lumen 236, and the leaflet perforation tool 200 may comprise a perforation tool guidewire 240 that extends through the lumen 236. In such examples, the guidewire 240 can be inserted into the patient's vasculature, and then the lacerating member 230 and/or the catheter 202 may be advanced toward the host leaflet 10 over the perforation tool guidewire 240.

In some examples, the perforation tool guidewire 240 can be used as a lacerating member for forming a pilot puncture 50. In such examples, the perforation tool guidewire 240 can be a relatively stiff wire having a distal tip configured to pierce the host leaflet 10 when the perforation tool guidewire 240 is pressed against the leaflet. Additionally, the perforation tool guidewire 240 can include a radio-frequency (RF) energy delivery tip 242 to assist with penetration through the leaflet tissue. For this purpose, a suitable RF energy device may be coupled to the perforation tool guidewire 240, and the RF energy device can apply the RF energy to the tip of the perforation tool guidewire 240 to penetrate the host leaflet 10. In any examples disclosed herein wherein a guidewire is used to puncture a leaflet, the guidewire can be coupled to a source of RF energy that applies RF energy to the tip of the guidewire. When the perforation tool guidewire 240 is used to pierce the leaflet, the lacerating member 230 in the form of a needle can be omitted.

In some examples, the lacerating member 230 in the form of a needle can be used in combination with the perforation tool guidewire 240 that forms an initial puncture in the leaflet. For example, the perforation tool guidewire 240 can be used to form an initial pilot puncture 50, after which the lacerating member 230 can be advanced through the leaflet to form a slightly larger pilot puncture for subsequent advancement of the expansion member 220 through the leaflet.

The expansion member 220 may include and/or be any suitable structure for expanding the pilot puncture 50 to form the leaflet opening 52. In some examples, the expansion member 220 extends at least substantially around a circumference of the catheter in order to exert an expansion force across full circumference of the pilot puncture 50. Additionally or alternatively, the expansion member 220 may have a circular profile when in the radially expanded configuration. This is not required of all examples, however, and it additionally is within the scope of the present disclosure that the expansion member 220 may have a non-circular profile when in the radially expanded configuration.

In some examples, and as shown in FIGS. 4A-4D, the expansion member 220 comprises an inflatable balloon 222 that is configured to be selectively inflated and deflated to transition the expansion member 220 between the radially compressed configuration and the radially expanded configuration. In particular, the inflatable balloon 222 may be selectively inflated to transition the inflatable balloon 222 from a deflated state (FIGS. 4A-4C) to an inflated state (FIG. 4D). Accordingly, the expansion member 220 is in the radially compressed configuration when the inflatable balloon 222 is in the deflated state, and the expansion member 220 is in the radially expanded configuration when the inflatable balloon 222 is in the inflated state.

FIGS. 4E-4H show optional subsequent steps in a method for implanting a replacement prosthetic valve inside the leaflet opening 52 formed by the lacerating member 230. As shown in FIG. 4E, the expansion member 220 can be recompressed after formation of the leaflet opening 52. In the case of an inflatable balloon 222, this can be achieved by deflating the balloon 222. In some examples, the guidewire 240 can be advanced to terminate distal to the host leaflet 10 either formation of the pilot puncture 50 by a separate lacerating member 230 that can be in the form of a needle, as illustrated in FIG. 4B, or independently of a separate needle, such as in a case where the guidewire 240 is configured to serve as a lacerating member, as described above and similar to the example illustrated in FIG. 4A. In some examples, the guidewire 240 can be advanced simultaneously with advancement of a lacerating member 230 that can be in the form of a needle towards the leaflet 14 and/or during formation of the pilot puncture 50. In some examples, the guidewire 240 can be advanced to terminate distal to the leaflet 10 during expansion of the expansion member (such as inflation of an inflatable balloon 222) or after recompression (such as by deflation) thereof.

Subsequent to recompressing the expansion member 220 inside the leaflet opening 52 and optionally advancing the guidewire 240 to extend therethrough, the tool catheter 202, optionally along with the remainder to the tool 200, can be retracted away from the host leaflet 10, as shown in FIG. 4F, leaving the guidewire 140 extending through the leaflet opening 52. The method can further include steps of positioning a replacement prosthetic valve 100, such as a guest prosthetic valve, inside the leaflet opening 52. A delivery apparatus carrying the guest prosthetic valve 100 can be either part of a system that includes the tool 200, or provided as a separate component advanced into a leaflet opening formed by a lacerating member 230 of the tool 200.

FIG. 4G shows a replacement prosthetic valve 100 positioned, in a radially compressed configuration thereof, inside the leaflet opening 52. The replacement prosthetic valve shown in FIGS. 4G-4F can be a second prosthetic valve 100^{b} that can be implanted inside a first (or host) prosthetic valve 100a, or a prosthetic valve 100 implanted inside a valvular structure 29 of a modified native heart valve 20. As shown in FIG. 4G, the prosthetic valve 100 can be mounted on a replacement valve delivery apparatus 280 that can be advanced towards the modified leaflet 14 over a guidewire, which can be a separate guidewire (not shown), or can be the same perforation tool guidewire 240.

In some examples, the replacement prosthetic valve is a balloon expandable heart valve, and the replacement valve delivery apparatus 280 comprises a balloon catheter 282 carrying a valve-expanding balloon 284. An inflatable balloon 222 of tool 200, which can be also termed a hole-dilating balloon, is different from a typical valve-expanding balloon 284 used for expanding balloon-expandable prosthetic valves or stents, in that while a typical valve-expanding balloon is inflatable to a diameter that can allow expansion of a prosthetic valve 100 to a functional diameter thereof, which can be similar to, or greater than (for example, in the case of valve over-expansion) the diameter of the native annulus in which the valve is deployed, the maximum diameter of a hole-dilating balloon 222 can be significantly smaller, configured to increase the size of a pilot puncture 50 to form a larger leaflet opening 52, optionally without tearing the host leaflet 10 (though in some examples, the leaflet 10 may be still torn by a balloon 222). In some examples, the maximum diameter to which the hole-dilating balloon 222 can be inflated is equal to or less than 14 mm. In some examples, the maximum diameter to which the hole-dilating balloon 222 can be inflated is equal to or less than 10 mm. In some examples, the maximum diameter to which the hole-dilating balloon 222 can be inflated is equal to or less than 12 mm. This may be in contrast to valve-expanding balloons conventionally inflatable to diameters of 23 mm or higher.

While a replacement valve delivery apparatus 280 equipped with a valve-expanding balloon 284 at a distal end portion of a balloon catheter 282 is illustrated, it is to be understood that this is shown by way of illustration and not limitation, and that a replacement valve delivery apparatus 280 can include other shafts and/or mechanisms, for example when utilized to advance and expand other types of replacement prosthetic valves, such as self-expandable prosthetic valves or mechanically expandable prosthetic valves.

In some examples, the replacement valve delivery apparatus 280 can further include a nosecone 286 positioned distal to the valve-expanding balloon 284 (or other prosthetic-valve expanding mechanism). The nosecone 286 can be conical or frustoconical in shape. The nosecone 286 can define a lumen through which a guidewire can extend. In some examples, when a nosecone 286 is present at a distal end of the replacement valve delivery apparatus 280 as also shown in the example illustrated in FIG. 4G, the nosecone 286 can be advanced towards the modified leaflet 14, and may optionally have a maximal diameter that can be somewhat greater than the diameter of the opening 52 formed by the lacerating member 230, such that as the nosecone 286 is inserted into the leaflet opening 52 it can optionally further expand the leaflet opening 52 to a greater diameter.

In some examples, the replacement valve delivery apparatus 280 and replacement prosthetic valve 100 are advanced towards and into the leaflet opening 52 of leaflet 10 over the same guidewire 240 used for advancement of the tool 200 towards the leaflet 14 for formation of the leaflet opening 52. As shown in FIG. 4G, the replacement prosthetic valve is placed in the leaflet opening 52 in its radially compressed configuration, optionally positioned over a deflated valve-expanding balloon 284 in the case of a balloon-expandable prosthetic valve.

With the guest prosthetic valve 100 received within the leaflet opening 52, radially expanding the prosthetic valve 100, as shown in FIG. 4H, can serve to increase a size of the leaflet opening 52 and/or to tear the leaflet. As a result, when the replacement prosthetic valve is a guest valve 100^{b} mounted inside a host prosthetic valve 100a, radially expanding the guest prosthetic valve can serve to modify the leaflet 10 such that the leaflet does not obstruct a cell opening 112 in a frame 102 of the host prosthetic valve 100a or at least increases the area of the host prosthetic valve 100a and the guest prosthetic valve 100^{b} that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt 118 of the guest prosthetic valve and/or away from one or more cell opening 112 of the guest prosthetic valve.

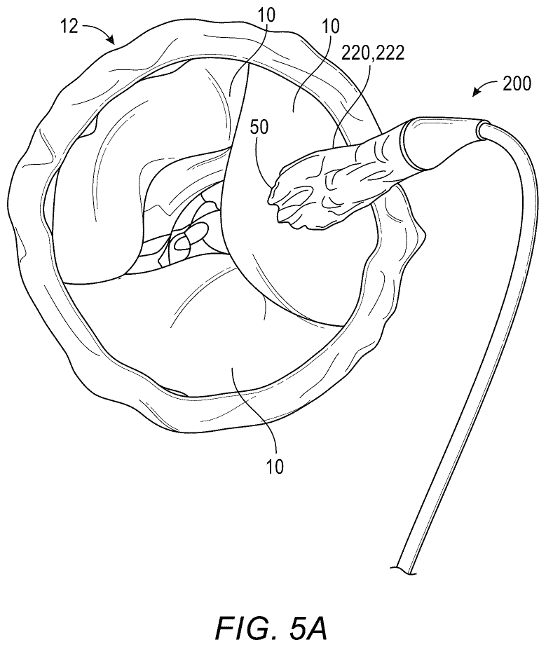

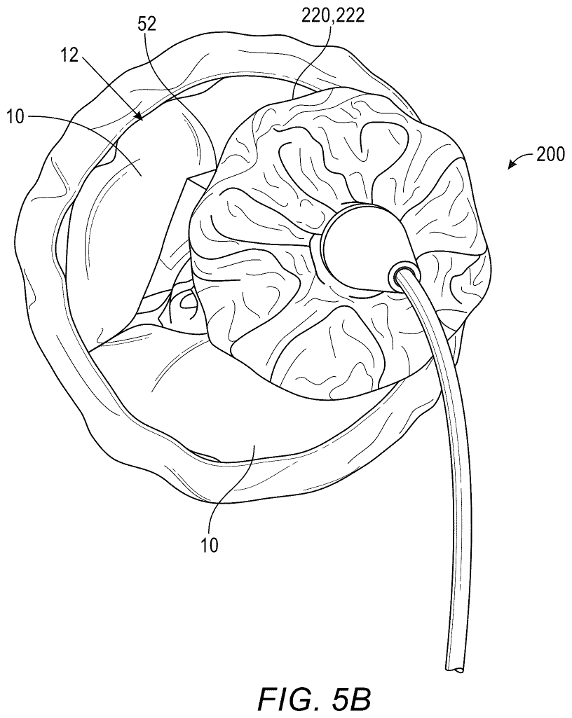

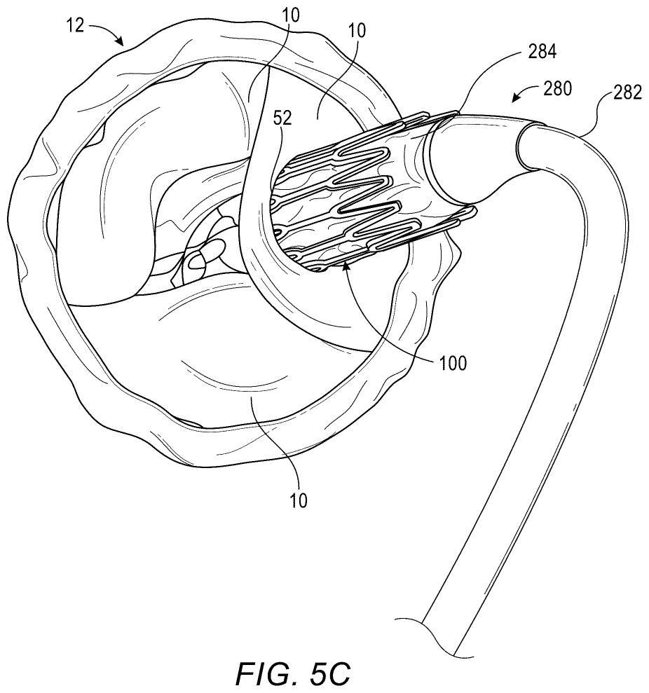

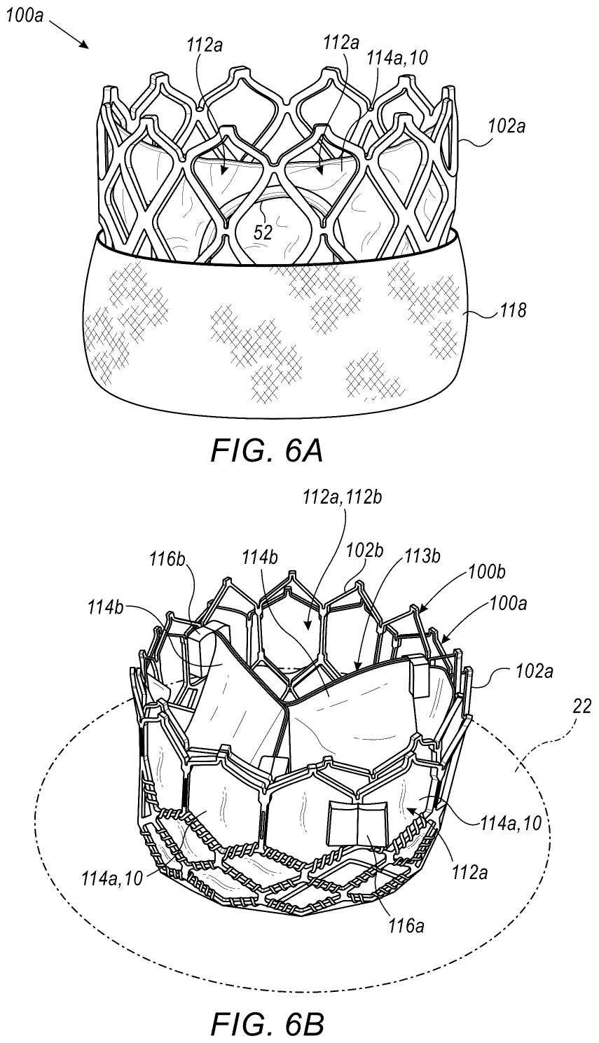

FIGS. 5A-6B illustrate a sequence of events in which a host valvular structure 12 is modified with the leaflet perforation tool 200 to receive a guest prosthetic valve 100. The host valvular structure 12 can be that of a host prosthetic valve 100a as illustrated in FIG. 3, or that of a native valve 20 as illustrated in FIGS. 2A-2B.

FIGS. 5A-5B illustrate a process by which an expansion member 220 in the form of an inflatable balloon 222 operates to expand the pilot puncture 50 into the leaflet opening 52. For example, FIG. 5A illustrates the hole-dilating inflatable balloon 222 in the deflated state within the pilot puncture 50, while FIG. 5B illustrates the inflatable balloon 222 in the inflated state such that the pilot puncture 50 has enlarged into the leaflet opening 52. FIG. 5C illustrated a guest prosthetic valve 100 that can be positioned in the leaflet opening 52 after removal of the inflatable balloon 222 therefrom, in a crimped configuration of the prosthetic valve, after which the prosthetic valve 100 can be expanded, such as by inflating a balloon over which it can be mounted in the case of a balloon-expandable prosthetic valve, so as to implant the guest prosthetic valve 100 inside the existing valvular structure.

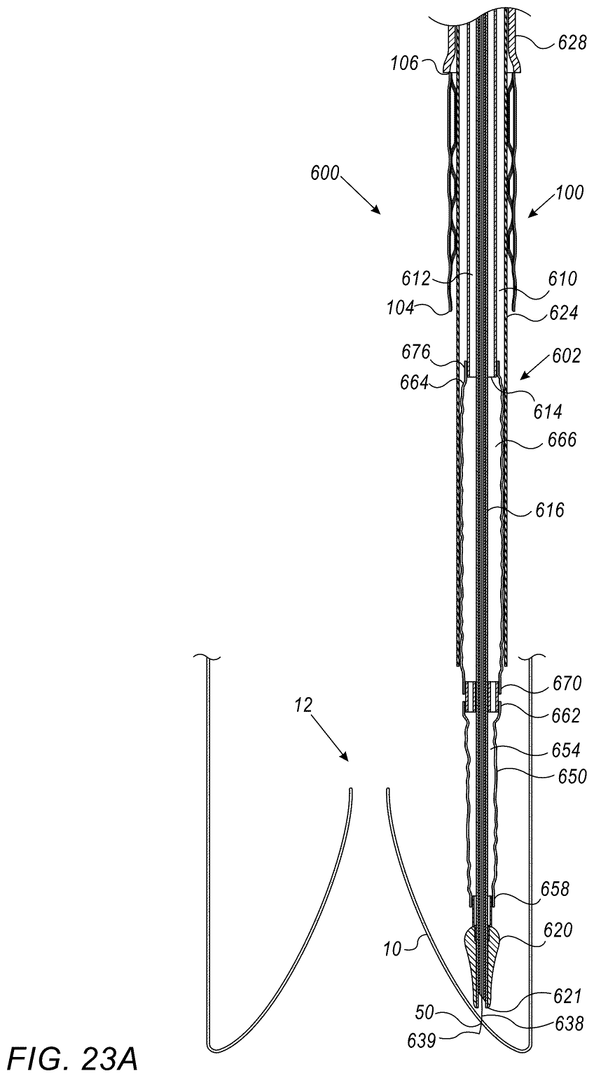

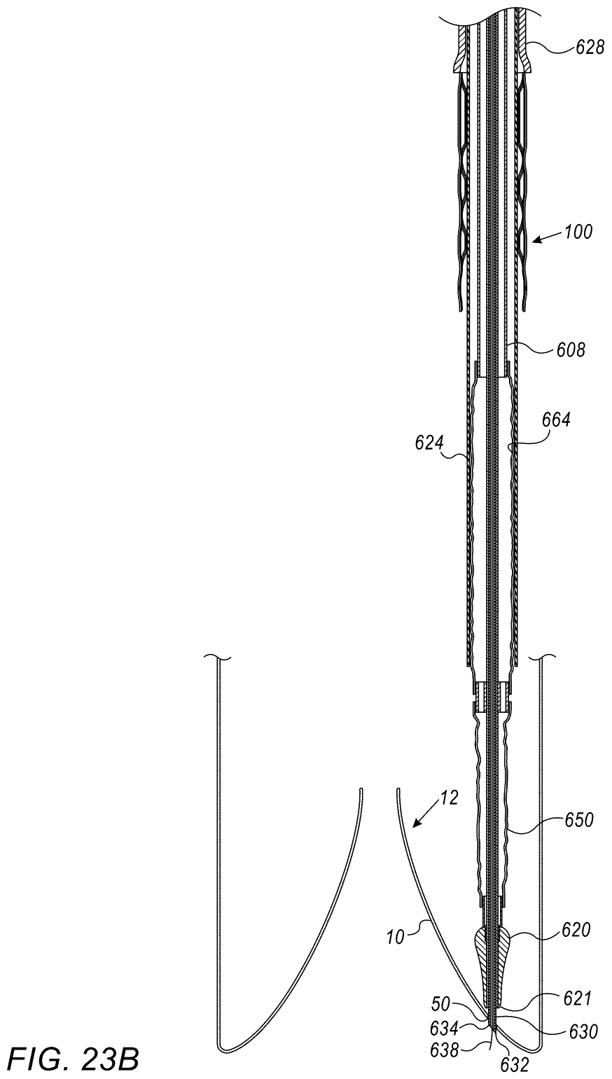

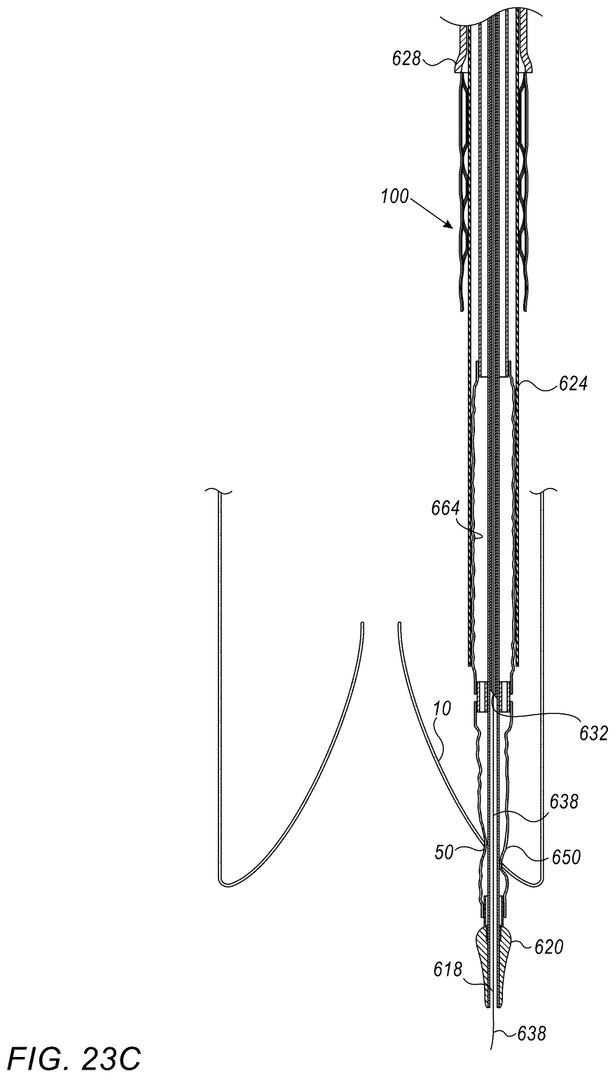

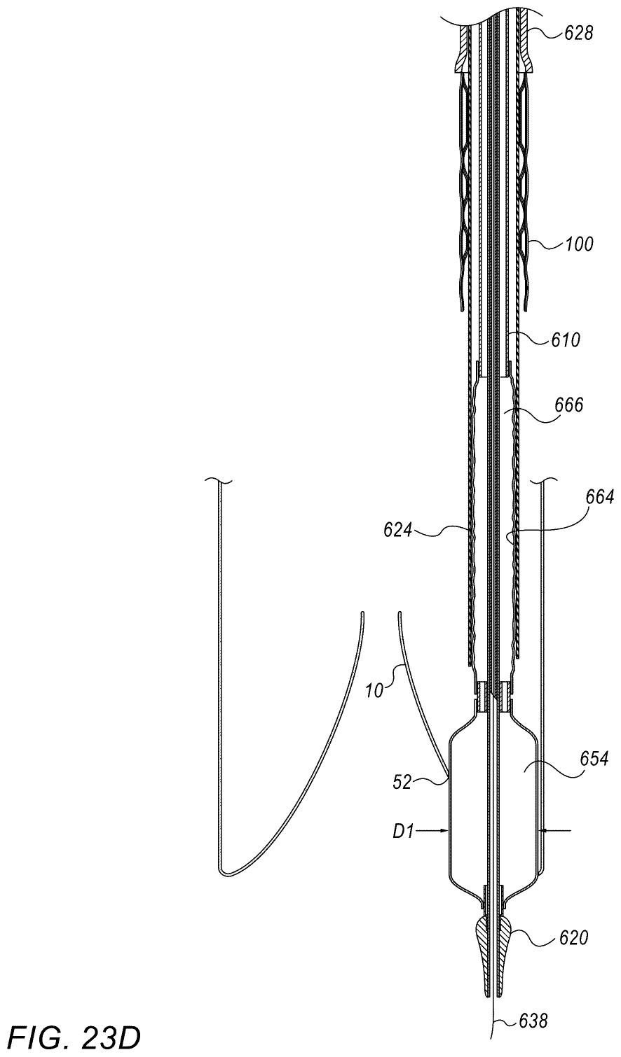

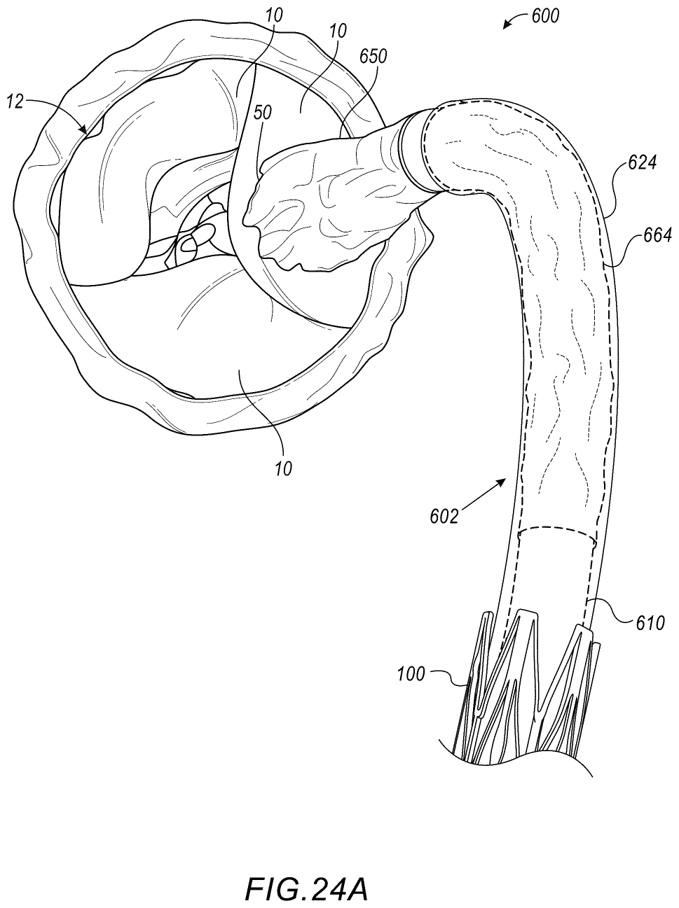

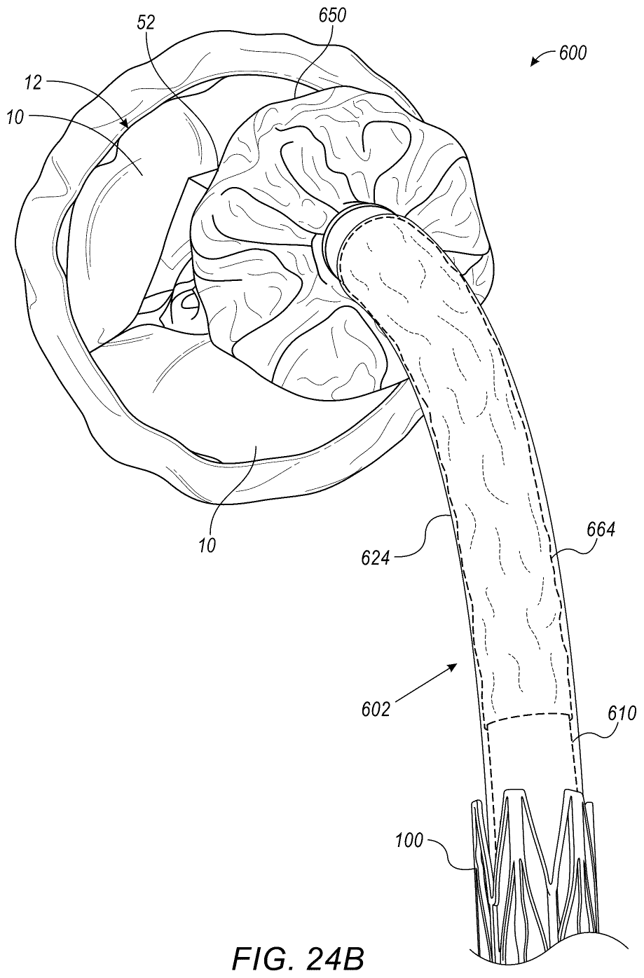

FIG. 6A shows a previously implanted prosthetic valve 100a subsequent to forming the leaflet opening 52, for example subsequent to the method described above with respect to FIGS. 4A-4E described above or any equivalent thereof, as well as FIGS. 23A-24B described below or any equivalents thereof. FIG. 6B shows a configuration in which a second prosthetic valve 100^{b} has been expanded within the leaflet opening 52 of a host prosthetic valve 100a. In the example of FIG. 6B, the guest prosthetic valve 100^{b} is the same type of valve as the host prosthetic valve 100a. It is to be understood, however, that the methods described herein, when implemented in ViV procedures, also may be applied to any other suitable valvular structures, such as different prosthetic valves and/or native heart valves. For example, the guest prosthetic valve 100^{b} need not be the same type of valve as the host prosthetic valve 100a.

In the example of FIG. 6A, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 6B, however, fully expanding the guest prosthetic valve 100^{b} within the leaflet opening 52 further expands and/or tears the leaflet opening 52 such that several cell openings 112a of the frame 102a of the host prosthetic valve 100a and several cell openings 112^{b} of the frame 102^{b} of the guest prosthetic valve 100^{b} are fully uncovered by the leaflets 114a. In some examples, this may result from the frame 102^{b} of the guest prosthetic valve 100^{b} pushing the leaflet 114a comprising the leaflet opening 52 downwardly (toward the inflow ends of the prosthetic valves 100a, 100^{b}) such that one or more cell openings 112a are unobstructed by the leaflet 114a. In some examples, expanding the frame 102^{b} within the leaflet 114a comprising the leaflet opening 52 may rip and/or tear this leaflet 114a such that the leaflet 114a cannot obstruct one or more cell openings 112a.

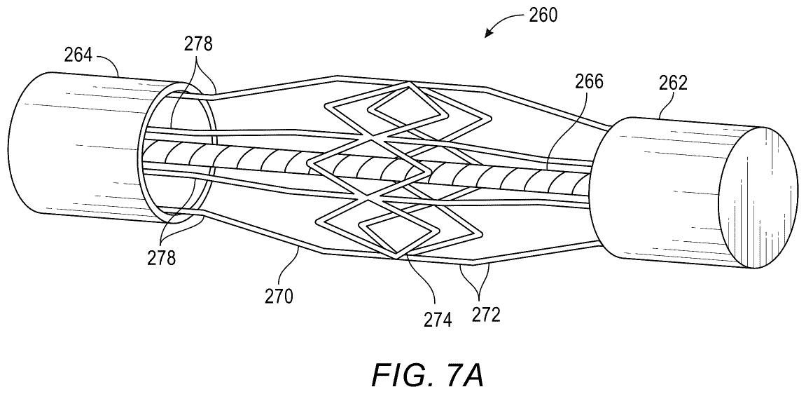

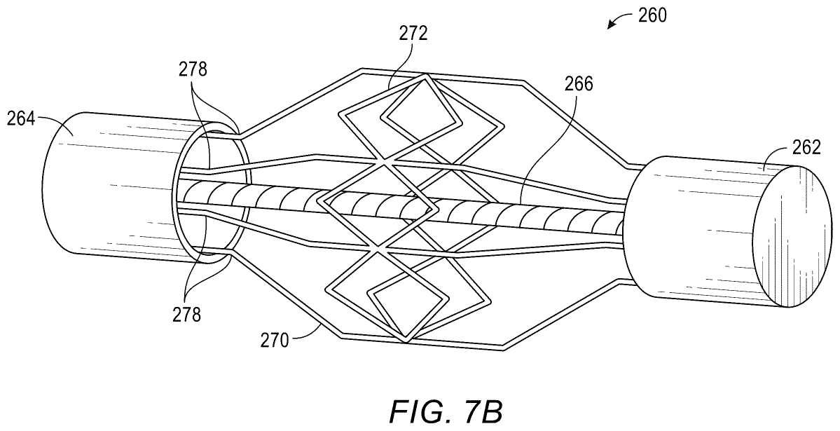

FIGS. 7A-7B illustrate another example of an expansion member 220 in the form of an expandable frame 260. The expandable frame 260 comprises a collapsible support structure 270 that is radially expandable from a first diameter (FIG. 7A) to a second diameter that is greater than the first diameter (FIG. 7B). For example, the collapsible support structure 270 can have the first diameter when the expandable frame 260 is in the radially compressed configuration, and the collapsible support structure 270 can have the second diameter when the expandable frame 260 is in the radially expanded configuration.

The collapsible support structure 270 can include a generally cylindrical expandable framework of frame members or struts 272. In some examples, plurality of frame members 272 are arranged and connected to define a geometrical structure that causes the collapsible support structure 270 to expand radially as the collapsible support structure 270 is compressed axially. For example, the frame members 272 can define substantially diamond shaped cells 274 that when axially compressed force the collapsible support structure 270 to expand radially.

When the expandable frame 260 transitions from the radially compressed configuration to the radially expanded configuration, the frame member 272 can exert a radially outwardly directed force on the leaflet 10 in which the expandable frame 260 is inserted, thereby expanding the pilot puncture 50 into the leaflet opening 52.

Because the frame members 272 are spaced apart from one another, using an expansion member 220 in the form of the expandable frame 260 may permit a flow of blood through the expandable frame 260 when the expandable frame 260 is in the radially expanded configuration. Accordingly, using an expansion member 220 in the form of an expandable frame 260 may obviate the rapid ventricular pacing that typically is performed during inflation of an inflatable balloon.

To transition the expandable frame 260 from the radially compressed configuration to the radially expanded configuration, the distance between a distal end portion 262 and a proximal end portion 264 of the expandable frame 260 can be selectively shortened. For example, and as shown in FIGS. 7A-7B, the expandable frame 260 can comprise a screw mechanism 266 that operatively interconnects the distal end portion 262 and the proximal end portion 264. For example, the screw mechanism 266 can have external threads that engage internal threads of the distal end portion 262 and/or the proximal end portion 264. In such examples, the screw mechanism 266 may be selectively rotated (for example, relative to the distal end portion 262, the proximal end portion 264, and/or the collapsible support structure 270) to cause distal end portion 262 to move closer to or further from the proximal end portion 264 (and/or vice-versa). In particular, rotating the screw mechanism 266 in a first direction can cause a distance between the distal end portion 262 and the proximal end portion 264 to decrease, thus forcing the frame members 272 to extend radially and transitioning the expandable frame 260 from the radially compressed configuration to the radially expanded configuration. Alternatively, rotating the screw mechanism 266 in a second direction opposite the first direction can cause the distance between the distal end portion 262 and the proximal end portion 264 to increase, thus forcing the frame members 272 to compress radially and transitioning the expandable frame 260 from the radially expanded configuration to the radially compressed configuration.

To facilitate the radial expansion of the frame members 272, the frame members can have hinge or bend areas 278 about which the frame members bend. The bend areas 278 can be pre-formed, notched, and/or otherwise configured such that frame members 272 will radially extend at the bend areas 278 when they are axially compressed.

The expandable frame 260 may be coupled to the catheter 202 in any of a variety of manners. In some examples, the proximal end portion 264 can be coupled to the distal end of the catheter 202, which can be configured to hold the frame 260 and prevent rotational movement of the frame relative to the screw mechanism 266 when the screw mechanism is rotated to expand the frame. The screw mechanism 266 can extend proximally through the lumen 208 of the catheter 202 and can have a proximal end portion operatively coupled to an actuator (for example, a rotatable knob) on the handle of the tool 200.

The leaflet perforation tool 200 also may include one or more features to facilitate positioning the leaflet perforation tool 200 relative to the leaflet 10 prior to and/or while forming the pilot puncture 50. For example, the leaflet perforation tool 200 may comprise one or more positioning arms supported by the catheter 202 and configured to facilitate positioning the catheter 202 relative to the host valvular structure.

In some examples, and as illustrated in dashed lines in FIG. 4A, the leaflet perforation tool 200 may comprise a first positioning arm 250 that is configured to engage a pocket 252 of the host valvular structure. The pocket 252 may be formed along a cusp edge of the leaflet. For example, the pocket 252 may be formed between a proximal surface of the leaflet 10 and a portion of the valvular structure radially exterior of the leaflet, such as an aortic wall or a frame of a previously implanted prosthetic valve. Because the location of the pocket 252 relative to a desired location of the pilot puncture 50 can be predetermined, positioning the first positioning arm 250 in the pocket 252 can provide a point of reference for locating the catheter 202 relative to the leaflet 10.

In some examples, and as illustrated in dashed lines in FIG. 4A, the leaflet perforation tool 200 may comprise a second positioning arm 254 that is configured to engage a free edge 256 of the leaflet 10. Because the location of the free edge 256 relative to a desired location of the pilot puncture 50 can be predetermined, positioning the second positioning arm 254 against the free edge 256 can provide a point of reference for locating the catheter 202 relative to the leaflet 10.

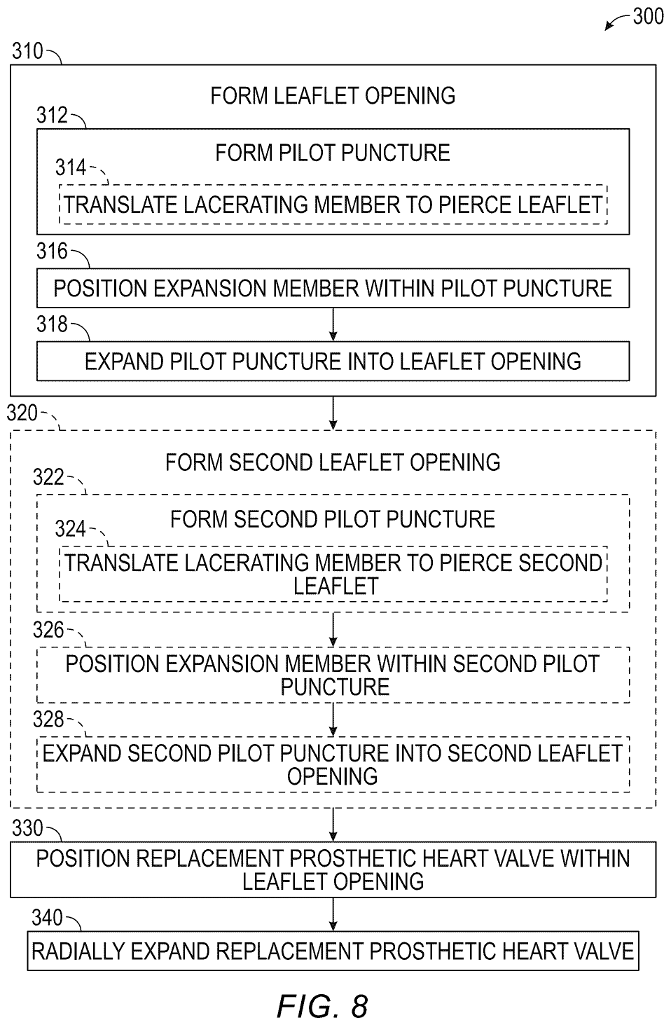

FIG. 8 is a flowchart depicting examples of a method 300 of implanting a replacement prosthetic valve within a host valvular structure. Examples of replacement prosthetic valves that may be utilized in conjunction with the method 300 are disclosed herein with reference to the prosthetic valve 100. The host valvular structure may include and/or be any of a variety of structures, examples of which include a native heart valve (for example, the native heart valve 20 disclosed herein) or a previously implanted prosthetic valve (for example, the first prosthetic valve 10a disclosed herein).

As shown in FIG. 8, the method 300 comprises forming, at 310, a leaflet opening within a leaflet of a host valvular structure. The forming the leaflet opening at 310 is performed with a leaflet perforation tool, such as the leaflet perforation tool 200 disclosed herein. The method 300 additionally comprises positioning, at 330, a replacement prosthetic valve in a radially compressed configuration within the leaflet opening and radially expanding, at 340, the replacement prosthetic valve.

The leaflet perforation tool used in the performance of the method 300 can comprise a catheter (such as the catheter 202 of the leaflet perforation tool 200) and an expansion member supported by the catheter (such as the expansion member 220 of the leaflet perforation tool 200).

As shown in FIG. 8, the forming the leaflet opening at 310 comprises forming, at 312, a pilot puncture within the leaflet and positioning, at 316, the expansion member within the pilot puncture. With the expansion member positioned within the pilot puncture, the method 300 further comprises transitioning, at 318, the expansion member from a radially compressed configuration to a radially expanded configuration to expand the pilot puncture into the leaflet opening. In the present disclosure, the transitioning the expansion member from the radially compressed configuration to the radially expanded configuration at 318 also may be referred to as expanding, at 318, the expansion member and/or as expanding, at 318, the pilot puncture into the leaflet opening.

In some examples, the method 300 additionally comprises, prior to the forming the pilot puncture within the leaflet at 312, positioning the leaflet perforation tool relative to the leaflet. For example, the method 300 may comprise positioning the leaflet perforation tool such that the laceration member is positioned to form the pilot puncture at a desired location within the leaflet. In particular, the leaflet perforation tool may comprise a first positioning arm supported by the catheter (such as the first positioning arm 250 disclosed herein) and/or a second positioning arm supported by the catheter (such as the second positioning arm 254 disclosed herein).

Accordingly, in some examples, the positioning the leaflet perforation tool relative to the leaflet can comprise positioning the catheter such that the first positioning arm engages a pocket formed along a cusp edge of the leaflet and/or between a proximal surface of the leaflet and a portion of the valvular structure radially exterior of the leaflet. In some examples, the positioning the leaflet perforation tool relative to the leaflet can comprise positioning the catheter such that the second positioning arm engages a free edge of the leaflet. In such examples, and as discussed above, the portions of the host valvular structure engaged by the first positioning arm and/or by the second positioning arm can provide a reference location relative to which the location of the catheter may be determined and/or adjusted.

The forming the pilot puncture at 312 may be performed in any of a variety of manners. In some examples, the leaflet perforation tool comprises a lacerating member, such as the lacerating member 230 of the leaflet perforation tool 200 disclosed herein, and the forming the pilot puncture at 312 is performed with the leaflet perforation tool. For example, the forming the pilot puncture at 312 can comprise translating the lacerating member in a distal direction relative to the catheter to pierce the leaflet to form the pilot puncture, as shown in FIGS. 4A 4A-2B.

After forming the pilot puncture, the catheter 202 can be advanced distally relative to the leaflet to position the expansion member 220 within the pilot puncture, as indicated at 316 and as shown in FIG. 4C. Thereafter, the expansion member 220 can be expanded to form the opening 52, as shown in FIG. 4D. In some examples, such as in the example of FIGS. 4C-4D discussed above, the method 300 can comprise retracting the lacerating member proximally away from the leaflet into the lumen 208 of the catheter 202 subsequent to the forming the pilot puncture at 312 and prior advancing the expansion member through the pilot puncture and expanding the expansion member. In some examples, the positioning the expansion member within the pilot puncture at 316 and/or the expanding the expansion member at 318 may be performed while the lacerating member extends through the leaflet.

The expanding the expansion member at 318 may be performed in any of a variety of manners. For example, the expansion member can comprise and/or be an inflatable balloon, such as the inflatable balloon 222 disclosed herein. In such examples, the expanding the expansion member at 318 can comprise inflating the inflatable balloon to transition the inflatable balloon from a deflated state (as shown in FIGS. 4A-4C and 5A) to an inflated state (as shown in FIGS. 4D and 5B).

In some examples, the expansion member can comprise and/or be an expandable frame, such as the expandable frame 260 disclosed herein. In such examples, the expanding the expansion member at 318 can comprise mechanically expanding the expandable frame, such as by actuating the screw mechanism 266 disclosed herein to radially expand a collapsible support structure of the expansion member. This is not required of all examples, however, and it also is within the scope of the present disclosure that any additional and/or alternative mechanisms may be used to transition the expansion member from the radially compressed configuration to the radially expanded configuration.

In some examples, one or more steps of the method 300 may be performed and/or facilitated with one or more guidewires. For example, positioning the leaflet perforation tool relative to the leaflet may comprise advancing the leaflet perforation tool toward the leaflet via a perforation tool guidewire, such as the perforation tool guidewire 240 disclosed herein. In some examples, the positioning the replacement prosthetic valve within the leaflet opening at 330 can comprise advancing the replacement prosthetic valve into the leaflet opening via a replacement valve guidewire. In some examples, the replacement valve guidewire and the perforation tool guidewire refer to the same guidewire. In some examples, the perforation tool guidewire extends alongside the replacement valve guidewire.

The radially expanding the replacement prosthetic valve at 340 may be performed in any suitable manner, such as using any suitable valve expansion technique and/or mechanism that is known to the art. For example, the radially expanding the replacement prosthetic valve at 340 can comprise inflating an inflatable balloon on which the replacement prosthetic valve is mounted.

In some examples, the replacement prosthetic valve can be a mechanically expandable prosthetic valve and the radially expanding the replacement prosthetic valve at 340 can comprise actuating a mechanical actuator of the replacement prosthetic valve to mechanically expand a frame of the replacement prosthetic valve.

In some examples, the replacement prosthetic valve can be a self-expandable prosthetic valve. When the replacement valve is a self-expandable prosthetic valve, the act of radially expanding the replacement valve can comprise advancing the replacement valve from a delivery capsule or otherwise removing a restraint from the replacement valve to allow it to radially self-expand within the leaflet opening.

With the replacement prosthetic valve received within the leaflet opening, the radially expanding the replacement prosthetic valve at 340 can serve to increase a size of the leaflet opening and/or to tear the leaflet. As a result, and as discussed above, the radially expanding the replacement prosthetic valve at 340 can serve to modify the leaflet such that the leaflet does not obstruct a frame opening in a frame of the replacement prosthetic valve or at least increases the area of the host valve and the guest valve that is not covered or obstructed by the modified leaflet to permit access and sufficient perfusion to the adjacent coronary artery. For example, radially expanding the replacement prosthetic valve within the leaflet opening can operate to push a portion of the leaflet extending radially exterior of the replacement prosthetic valve below an upper edge of an outer skirt of the replacement prosthetic valve and/or away from one or more frame openings of the replacement prosthetic valve.

In some examples, the method 300 can comprise repeating one or more steps disclosed above to form a plurality of punctures and openings in the host valvular structure. For example, the forming the leaflet opening at 310 can comprise forming a first leaflet opening in a first leaflet, the method 300 further can comprise forming, at 320, a second leaflet opening within a second leaflet of the host valvular structure. The forming the second leaflet opening at 320 can comprise forming, at 322, a second pilot puncture within the second leaflet and positioning, at 326, the expansion member within the second pilot puncture.

With the expansion member positioned within the second pilot puncture, the method 300 further can comprise transitioning, at 328, the expansion member from the radially compressed configuration to the radially expanded configuration to expand the second pilot puncture into the second leaflet opening.

In some examples, forming the second leaflet opening can provide further access and/or fluid paths through the frame of the replacement prosthetic valve. For example, radially expanding the replacement prosthetic valve within the first leaflet opening may push the second leaflet against the frame of the replacement prosthetic valve such that the second leaflet opening is aligned with a frame opening of the frame of the replacement prosthetic valve. Thus, the second leaflet opening can provide additional unobstructed paths through the frame of the replacement prosthetic valve. Moreover, in an example in which the host valvular structure is a previously implanted prosthetic valve, expanding the replacement prosthetic valve within the first leaflet opening can trap the second leaflet opening between the respective frames of the previously implanted prosthetic valve and the replacement prosthetic valve, thereby providing additional access and/or flow paths through each of the frames.

Thus, forming the second leaflet opening can ensure that a greater number of frame openings of the frame are uncovered, and/or that a greater proportion of the frame is uncovered, relative to an example in which only one leaflet is punctured to form a leaflet opening. This may be particularly beneficial in examples in which the frame of a previously implanted prosthetic valve extends axially in a downstream direction beyond one or both of the coronary arteries when the replacement prosthetic valve is implanted within a native heart valve.

For example, in some patient anatomies, the left coronary artery is positioned lower (that is, proximate to the host valvular structure) than the right coronary artery. In such examples, the right coronary artery may be sufficiently far from the host valvular structure that implanting the replacement prosthetic valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In some examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic valve may ensure the ability for future access into both coronary arteries or perfusion through the frame to both coronary arteries during the diastole phase of the cardiac cycle. In some examples, the host valvular structure can be modified such that the replacement prosthetic valve is implanted a first leaflet that faces the left coronary artery, and such that the second leaflet opening is formed in a second leaflet that faces the right coronary artery (or vice-versa).

The forming the second pilot puncture at 322 may be performed in any of a variety of manners. For example, the forming the second pilot puncture at 322 can comprise translating, at 324, the lacerating member in a distal direction relative to the catheter to pierce the second leaflet to form the second pilot puncture.

In some examples, the forming the first leaflet opening at 310 can be performed prior to the forming the second leaflet opening at 320. In some examples, the forming the second leaflet opening at 320 can be performed prior to the forming the first leaflet opening at 310.

In an example in which the method 300 comprises the forming the first leaflet opening at 310 and the forming the second leaflet opening at 320, the positioning the replacement prosthetic valve at 330 may comprise positioning the replacement prosthetic valve in the first leaflet opening. Accordingly, in such examples, the radially expanding the replacement prosthetic valve at 340 can comprise radially expanding the replacement prosthetic valve within the first leaflet opening. This is not required, however, and it additionally is within the scope of the present disclosure that the replacement prosthetic valve can be positioned within and expanded within the second leaflet opening.

Moreover, in the method 300, the replacement prosthetic valve need not be implanted within an opening 52 of a leaflet. For example, in cases where the expansion member forms a full tear in a leaflet that extends to the coaptation edge of the leaflet, the replacement prosthetic valve can be positioned at a location between the leaflets of the host valvular structure (such that the delivery device used to implant to replacement prosthetic valve does not necessarily extend through the tear) and then expanded. In some examples, such as in cases where the opening 52 does not form a full tear in the leaflet, the replacement prosthetic valve can be positioned at a location between the leaflets of the host valvular structure (such that the delivery device used to implant to replacement prosthetic valve does not extend through the opening) and then expanded. In such cases, the opening 52 may provide sufficient open space through which blood may flow into the coronary ostia, and/or through which additional access devices, such as coronary catheters, can pass during future interventional procedures.

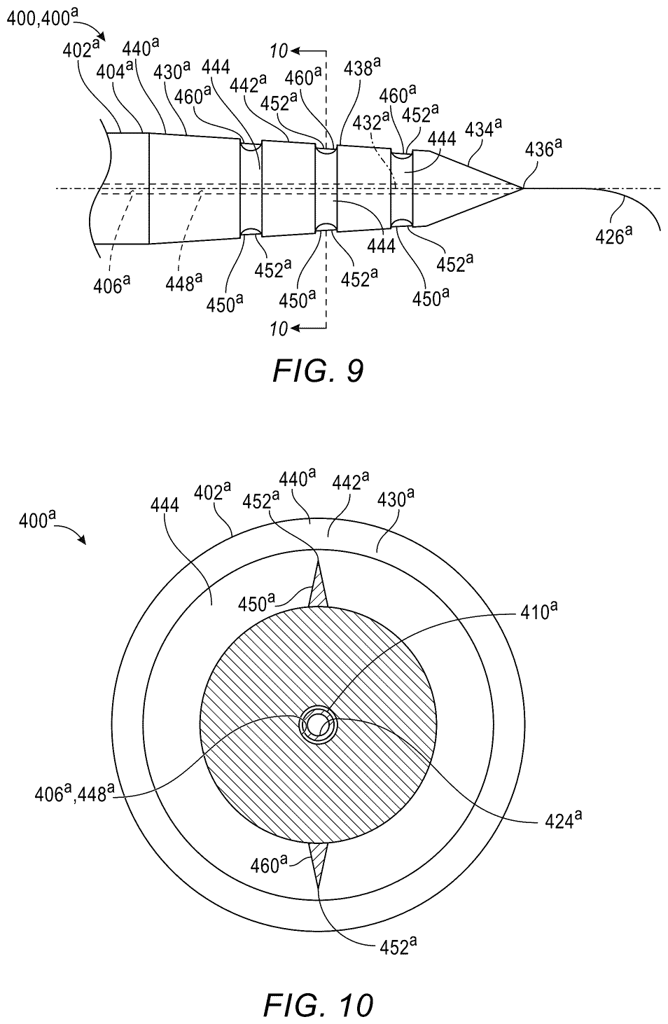

FIGS. 9-12B illustrate another example of a leaflet perforation tool 400 that may be used to form a leaflet opening in a leaflet of a host valvular structure. The host valvular structure may be any of a variety of valvular structures, such as a valvular structure of a native heart valve, such as an aortic valve (for example, the native heart valve 20), a mitral valve, a pulmonary valve, or a tricuspid valve, or a valvular structure of a previously implanted prosthetic valve that is implanted within a native heart valve (for example, the prosthetic valve 100). Additionally, forming the leaflet opening with the leaflet perforation tool 400^{a} may be performed for any purpose and/or to achieve any effect disclosed herein with reference to the leaflet perforation tool 200.

Various exemplary implementations for leaflet perforation tools 400 can be referred to, throughout the specification, with superscripts, for ease of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of a perforation tool 200 or a component thereof, referred to with a superscript, may be optionally shared by some but not necessarily all other exemplary implementations. FIGS. 9-12B show a leaflet perforation tool 400^{a} which is an exemplary implementation of leaflet perforation tool 400. As shown in FIG. 9, a leaflet perforation tool 400^{a} comprises a catheter 402^{a} with a distal end portion 404^{a} configured to be inserted into a patient's vasculature and a nosecone 430a coupled to the distal end portion 404^{a} of the catheter 402^{a}. The nosecone comprises a main body 440^{a} and at least one blade 450^{a} coupled to the main body 440^{a}. As described in more detail below, the nosecone 430^{a} is configured to be inserted through a leaflet of a host valvular structure to form a leaflet opening within the leaflet.

As shown in FIG. 9, at least a portion of the nosecone 430^{a} may be substantially conical in shape. FIG. 9 illustrates an example in which the nosecone 430^{a} comprises a distal portion 434^{a} that is at least substantially conical in shape and a proximal portion 438^{a} that is at least substantially frustoconical in shape. The distal portion 434^{a} and the proximal portion 438^{a} may have different shapes. For example, the distal portion 434^{a} may taper toward a central axis 432a of the nosecone 430^{a} more steeply than does the proximal portion 438^{a}. The distal portion 434^{a} can form a pointed tip at the distal end of the nosecone 430a.

The generally conical shape of the nosecone 430^{a} can facilitate forming the leaflet opening in the leaflet. For example, advancing the nosecone 430^{a} through the leaflet (for example, through a pilot opening formed in the leaflet) can force the leaflet opening to expand in diameter with the increasing diameter of the portion of the nosecone 430^{a} received within the leaflet.

In some examples, at least a portion of the nosecone 430^{a} is flexible (for example, formed of a flexible material, such as natural rubber or any of various synthetic elastomers) to facilitate navigating the leaflet perforation tool 400^{a} through the patient's vasculature. Additionally or alternatively, and as shown in FIG. 9, the nosecone 430^{a} can comprise a lumen 448^{a} that receives a perforation tool guidewire 426^{a} of the leaflet perforation tool 400^{a}. In such examples, the perforation tool guidewire 426^{a} can be used to guide the leaflet perforation tool 400^{a} through the patient's vasculature and/or toward the leaflet to be punctured. As shown in FIG. 9, the catheter 402^{a} also can comprise a lumen 406^{a} that is connected to the lumen 448^{a} of the nosecone 430^{a}. Thus, in such examples, the perforation tool guidewire 426 can extend through each of the lumen 406^{a} and the lumen 448^{a}. In some examples, the lumen 406^{a} and the lumen 448^{a} can refer to respective portions of a single continuous lumen.

Each blade 450^{a} may facilitate forming and/or expanding the leaflet opening as the nosecone 430^{a} is advanced through the leaflet, such as by cutting a portion of the leaflet adjacent to the leaflet opening. As shown in FIG. 9, each blade 450^{a} can comprise (for example, can terminate at) a respective blade edge 452^{a} that extends along a direction that is at least substantially coplanar with the central axis 432^{a} of the nosecone 430^{a} and/or that is at least substantially parallel to the central axis 432^{a}. In some examples, the blade edge 452^{a} can be angled relative to the central axis 432^{a} of the nosecone 430^{a}. In some examples, an acute angle can be defined between the direction along which the blade edge 452^{a} extends and the central axis 432^{a} of the nosecone 430a.

To protect the patient's anatomy from each blade 450a prior to advancing the nosecone 430^{a} through the leaflet, each blade 450^{a} can be at least partially shielded and/or obscured by the main body 440^{a} of the nosecone 430^{a}. For example, and as shown in FIG. 9, each blade 450^{a} can be positioned fully radially inward of an external surface 442^{a} of the main body 440^{a}. As used herein, the term “external surface” with reference to the main body 440^{a} is intended to refer to the portion of the outer surface of the main body 440^{a} that is coextensive with an envelope representing the outermost shape of the main body 440a. For example, in the example of FIG. 9, the external surface 442^{a} refers to the portions of the outer surface of the main body 440^{a} that form conical or frustoconical surface.

As shown in FIG. 9, the nosecone 430^{a} additionally comprises at least one groove 444 extending at least partially circumferentially around the nosecone 430^{a} (for example, around the central axis 432^{a}) and recessed toward the central axis 432^{a}. In the example of FIG. 9, each groove 444 extends fully circumferentially around the nosecone 430^{a}. FIG. 9 illustrates an example in which the nosecone 430^{a} comprises a plurality of grooves 444 (such as three grooves 444 in the illustrated example) that are axially offset from one another. It is to be understood, however, that the nosecone 430^{a} also can comprise any suitable number of grooves 444, such as one groove 444, two grooves 444, four grooves 444, or more than four grooves 444.

The surface of the main body 440^{a} within each groove 444 may be described as being distinct from, and/or as being recessed relative to, the external surface 442^{a} of the main body 440^{a}. FIG. 10 is a cross-sectional view of the leaflet perforation tool 400^{a} as viewed along the line 10-10 of FIG. 9, which runs through a selected groove 444.

For each groove 444, and as shown in FIGS. 9-10, a first blade 450^{a} can be positioned within the groove 444 (for example, within a first groove 444) such that the blade edge 452^{a} of the first blade 450^{a} is recessed relative to the external surface 442^{a} of the main body 440a.

In some examples, the nosecone 430^{a} comprises a plurality of blades 450^{a}. For example, in the example of FIGS. 9-10, a second blade 460^{a} also is positioned within the groove 444 in a similar manner as the first blade 450^{a} and circumferentially offset from the first blade 450^{a}. In this manner, positioning the first blade 450^{a} and the second blade 450^{b} within the groove 444 may protect each blade edge 452^{a} from inadvertently contacting native tissue other than the intended host leaflet.

In the example of FIGS. 9-10, the first blade 450^{a} and the second blade 460^{a} within the groove 444 are circumferentially offset from one another by an angle that is about 280 degrees. It is to be understood, however, that the first blade 450^{a} and the second blade 460^{a} may be circumferentially offset by any suitable angle, and/or that the groove 444 may receive more than two blades 450^{a}. In some examples, each groove 444 can receive more than two blades 450a.

In some examples, the grooves 444 need not extend completely around the nosecone 430^{a}. That is, the groove 444 can have a circumferential dimension that is less than 360 degrees. Where multiple blades 450a, 460^{a} are provided at a particular axial location along the length of the nosecone, there can be multiple such grooves that are discontinuous or circumferentially spaced from each other, with a blade disposed in each groove. If only one blade 450^{a} is provided at any axial location along the length of the nosecone, the groove can have a circumferential dimension that is less than 360 degrees.

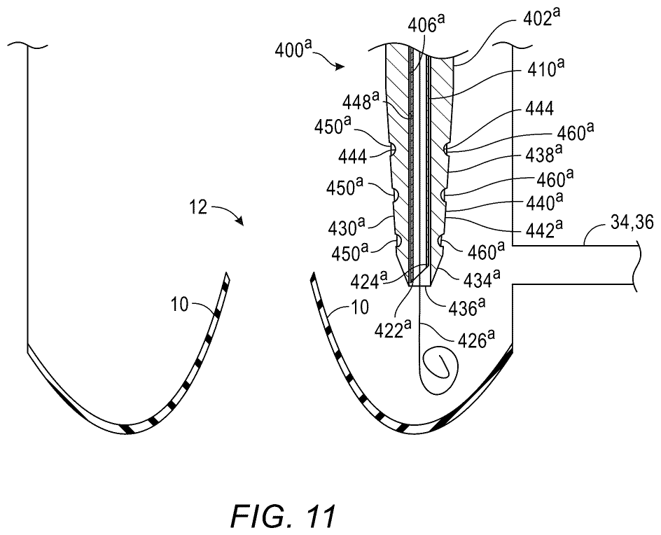

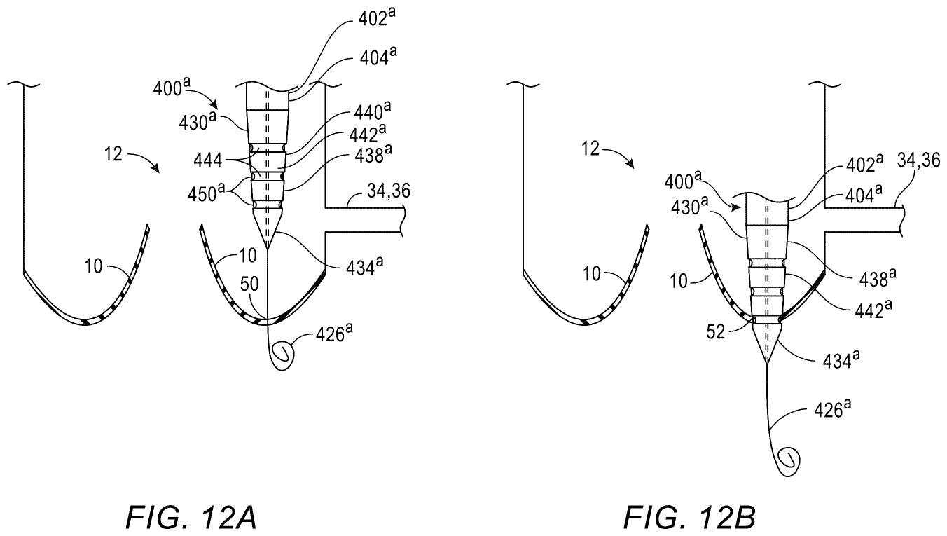

FIG. 11 shows the leaflet perforation tool 400^{a} in cross-section and positioned adjacent to a host valvular structure 12. FIGS. 12A-12B show a process by which the leaflet perforation tool 400^{a} may be used to form a leaflet opening in a leaflet 10 of the host valvular structure 12. Thus, FIGS. 11-12B may be described as representing an example in which the leaflet perforation tool 400^{a} forms a leaflet opening in the host leaflet 10 of the host valvular structure 12 for receiving a guest prosthetic valve 100 therein. Descriptions of the leaflet perforation tool 400^{a} piercing and/or extending through the host leaflet 10 also may applied to examples in which the leaflet perforation tool 400^{a} pierces and/or extends through a native leaflet 30 of a native heart valve 20 or a prosthetic valve leaflet 114 of a previously implanted prosthetic valve 100a.

As shown in FIGS. 10-11, the leaflet perforation tool 400^{a} optionally can comprise a lacerating member 410^{a} that is configured to pierce the leaflet 10 to form a pilot puncture 50 in the leaflet 10. After forming the pilot puncture 50, and as shown in FIG. 12B, the nosecone 430a can be inserted into the pilot puncture 50 to expand the pilot puncture 50 to form a leaflet opening 52.

In some examples, the lacerating member 410^{a} may include and/or be a needle, such as a spring-loaded needle and/or a Veress needle. Additionally or alternatively, the lacerating member 410^{a} may share any suitable features or properties with the lacerating member 230 of the leaflet perforation tool 200 described above.

As shown in FIGS. 10-11, the lacerating member 410^{a} can be disposed within the lumen 448^{a} of the nosecone 430^{a}. The lacerating member 410^{a} can comprise a distal end 422^{a} that is configured to be selectively translated in the proximal direction and in the distal direction relative to a distal end 436^{a} of the nosecone. In some examples, and as shown in FIG. 11, the lacerating member 410^{a} comprises a lumen 424^{a} that receives the perforation tool guidewire 426^{a}.

In other examples, the pilot puncture 50 can be formed using a guidewire, such as the perforation tool guidewire 426^{a}. In such examples, the lacerating member 410^{a} in the form of a needle can be omitted entirely, or can be used in combination with the perforation tool guidewire 426^{a} or another guidewire to form the pilot puncture, as previously discussed for the tool 200. In examples in which the lacerating member 410^{a} in the form of a needle is omitted, the perforation tool guidewire 426^{a} can be used as the lacerating member. FIG. 12A illustrates an example in which the perforation tool guidewire 426^{a} punctures the leaflet 10 to form the pilot puncture 50 within the leaflet 10 such that the nosecone 430^{a} subsequently may be inserted into the pilot puncture 50 (as shown in FIG. 12B). In some examples, the perforation tool guidewire 426^{a} can be used to deliver the tool 400^{a} such that the nosecone is at a location adjacent the native valve, after which the perforation tool guidewire 426^{a} can be removed and replaced with a relatively stiffer guidewire and/or a guidewire with a substantially straight end portion (for example, without a pig tail) to facilitate puncturing the leaflet 10.

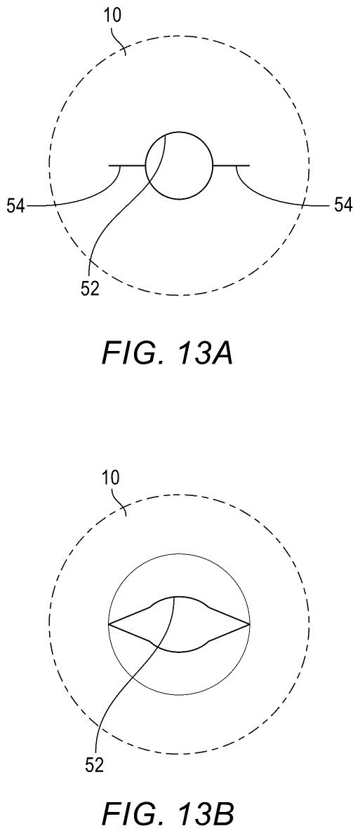

FIGS. 12A-13B illustrate a manner in which the blades 450^{a} of the nosecone 430^{a} can facilitate forming the leaflet opening 52. As shown in FIG. 12B, as the nosecone 430^{a} is inserted into the leaflet 10, the inherent resiliency of the leaflet 10 may urge the leaflet 10 radially inwardly against the nosecone 430a. When each groove 444 passes through the opening in the leaflet 10, the leaflet 10 thus extends into the groove 444 and contacts the blade(s) 450a positioned therein. With reference to FIG. 13A, each blade 450^{a} thus forms a leaflet slit 54 within the leaflet 10 that is connected to the leaflet opening 52.

The leaflet slits 54 thus can facilitate expanding the leaflet opening 52 to a greater diameter. For example, and as shown in FIG. 13B, advancing an implant or other type of medical device (such as the replacement prosthetic valve 10^{b}) through the leaflet opening 52 can cause the leaflet slits 54 to open, thus readily expanding the leaflet opening 52 to accommodate the medical device.

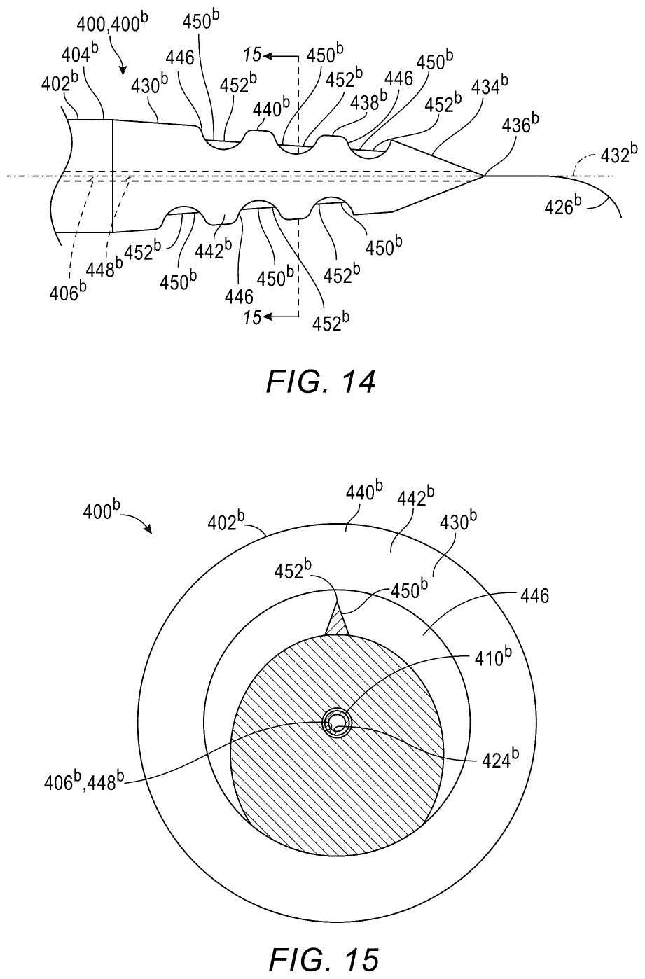

FIGS. 14-16B illustrate a leaflet perforation tool 400^{b}, which is an example of leaflet perforation tool 400 and may thus share common features of the leaflet perforation tool 400. The leaflet perforation tool 400^{b} can exhibit any suitable properties, features, etc. disclosed herein with reference to the leaflet perforation tool 400^{a}. The leaflet perforation tool 400^{b} differs from the leaflet perforation tool 400a, however, at least in the manner in which the blades 450^{b} are arranged relative to one another.

As shown in FIG. 14, the nosecone 430^{b} of the leaflet perforation tool 400^{b} can comprise at least one groove 446 extending partially circumferentially around the nosecone 430^{b}, and each blade 450^{b} of the nosecone 430^{b} can be positioned within a respective groove 446. Similar to the nosecone 430^{a} of the leaflet perforation tool 400^{a}, each blade 450^{b} can be received within the respective groove 446 such that the blade 450^{b} is positioned fully radially inward of the external surface 442^{b} of the main body 440^{b}, and/or such that the respective blade edge 452^{b} is recessed relative to the external surface 442^{b}.

In the example of FIG. 14, the nosecone 430^{b} comprises a plurality of grooves 446, and each blade 450^{b} is received within a respective groove 446. The nosecone 430^{b} can be configured such that two grooves 446 of the plurality of grooves 446 (for example, two axially adjacent grooves 446) are positioned such that the respective blades 450^{b} positioned within the grooves 446 are circumferentially offset from one another by an angle that is about 280 degrees.

In some examples, the grooves 446 and/or the respective blades 450^{b} can be circumferentially offset by any other suitable angle. For example, the grooves 446 can be arranged such that the respective blades 450^{b} positioned in a pair of axially adjacent grooves 446 are circumferentially offset from one another by less than 45 degrees, about 45 degrees, about 90 degrees, about 220 degrees, or more than 220 degrees.

As shown in FIG. 14, the plurality of grooves 446 may be at least partially axially offset from one another such that at least a portion of each blade 450^{b} is axially offset from each other blade 450^{b}. Stated differently, for each blade 450^{b} in such an example, there can exist a plane extending perpendicular to the central axis 432^{b} that intersects that blade 450^{b} and no other blades 450^{b}. FIG. 15 is a cross-sectional view of the leaflet perforation tool 400^{b} as cut along such a plane, and in particular a plane extending along the line 15-15 of FIG. 14.

As shown in FIG. 15, the leaflet perforation tool 400^{b} optionally can comprise a lacerating member 410^{b} that is configured to pierce the leaflet 10 to form a pilot puncture 50 in the leaflet 10, as discussed above with respect to the lacerating member 410^{a} of the leaflet perforation tool 400a. In particular, and as shown in FIG. 15, the lacerating member 410^{b} can be disposed within the lumen 448^{b} of the nosecone 430^{b}. The lacerating member 410^{b} can comprise a lumen 424^{b} that receives the perforation tool guidewire 426^{b}.

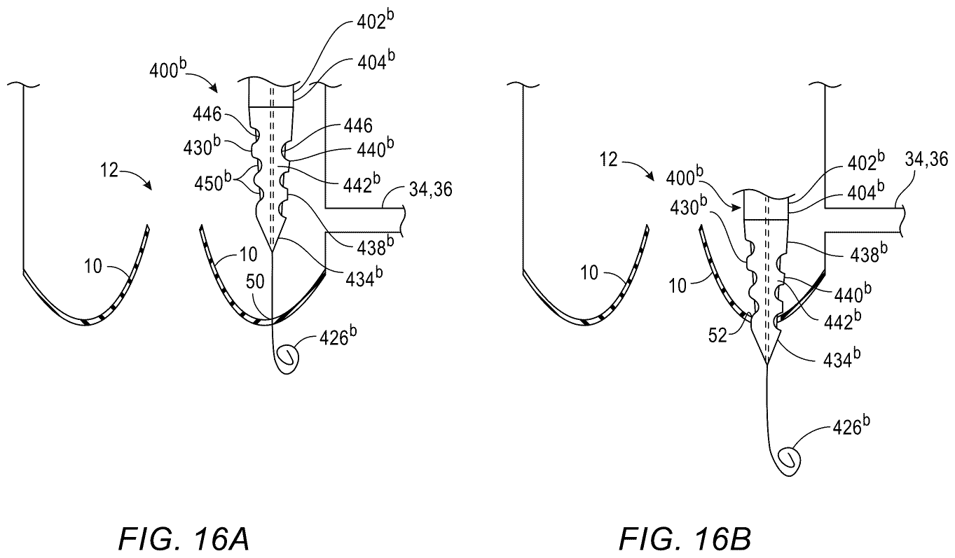

In some examples, and as discussed above with respect to the leaflet perforation tool 400^{a}, the pilot puncture 50 can be formed using a guidewire, such as the perforation tool guidewire 426^{b}. In such examples, the lacerating member 410^{b} in the form of a needle can be omitted entirely, or can be used in combination with the perforation tool guidewire 426^{b} or another guidewire to form the pilot puncture. In examples in which the lacerating member 410^{b} in the form of a needle is omitted, the perforation tool guidewire 426 can be used as the lacerating member. FIG. 16A illustrates an example in which the perforation tool guidewire 426^{b} punctures the leaflet 10 to form the pilot puncture 50 within the leaflet 10 such that the nosecone 430^{b} subsequently may be inserted into the pilot puncture 50 (as shown in FIG. 16B). In some examples, the perforation tool guidewire 426^{b} can be used to deliver the tool 400^{b} such that the nosecone is at a location adjacent the native valve, after which the perforation tool guidewire 426^{b} can be removed and replaced with a relatively stiffer guidewire and/or a guidewire with a substantially straight end portion (for example, without a pig tail) to facilitate puncturing the leaflet 10.

FIGS. 16A-16B show a process by which the leaflet perforation tool 400^{b} may be used to form a leaflet opening in the host leaflet 10 of the host valvular structure 12. As shown in FIG. 16A, the perforation tool guidewire 426^{b} of the leaflet perforation tool 400^{b} can be used to form the pilot puncture 50 in the leaflet 10. After forming the pilot puncture 50, and as shown in FIG. 16B, the nosecone 430^{b} can be inserted into the pilot puncture 50 to expand the pilot puncture 50 to form the leaflet opening 52. The resulting leaflet opening 52 can be substantially similar to that shown in FIGS. 13A-13B, including the leaflet slits 54 formed by the blades 450^{b}.

As discussed above, when the nosecone 430^{b} is inserted through the leaflet 10, the leaflet 10 may be urged radially inwardly against the nosecone 430^{b}. Due to the axially offset configuration of the blades 450^{b}, and as shown in FIG. 16B, the nosecone 430^{b} can be inserted to an axial position such that the leaflet 10 contacts only one blade 450^{b}. In such an orientation, the portion of the leaflet 10 circumferentially opposite the contacted blade 450^{b} engages the external surface 442^{b} of the main body 440^{b}, which in turn can exert a radially outwardly directed force upon the leaflet 10 to draw the leaflet 10 into closer engagement with the blade 450^{b}. Accordingly, such a configuration may facilitate cutting the leaflet 10 with the blade 450^{b} relative to a configuration in which the blade 450^{a} is circumferentially opposite (and axially aligned with) a second blade 460a.

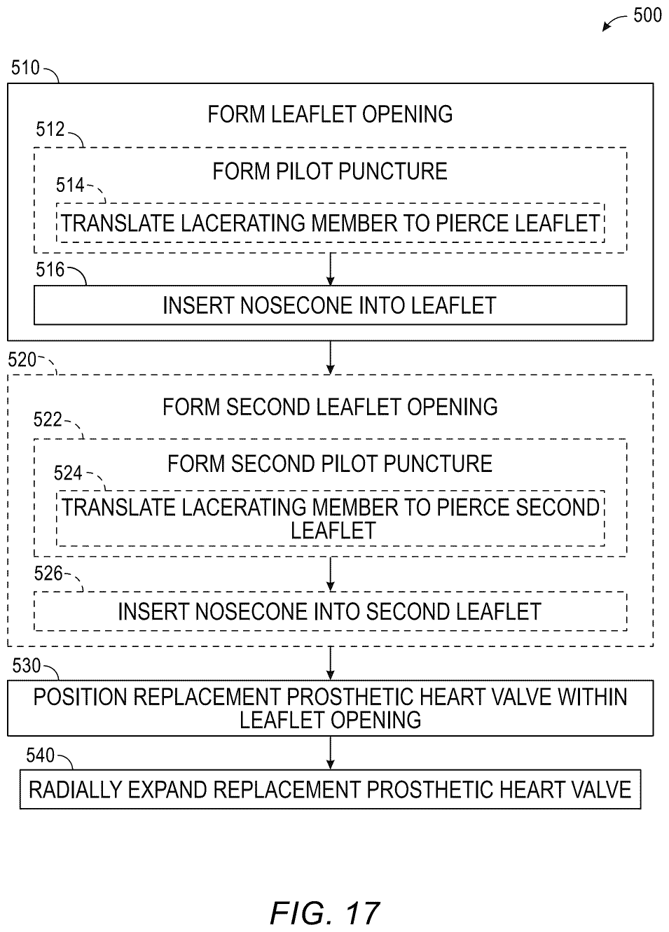

FIG. 17 is a flowchart depicting examples of a method 500 of implanting a replacement prosthetic valve within a host valvular structure. Examples of replacement prosthetic valves that may be utilized in conjunction with the method 500 are disclosed herein with reference to the prosthetic valve 100. The host valvular structure may include and/or be any of a variety of structures, examples of which include a native heart valve (for example, the native heart valve 20 disclosed herein) or a previously implanted prosthetic valve (for example, the first prosthetic valve 100a disclosed herein).

As shown in FIG. 17, the method 500 comprises forming, at 510, a leaflet opening within a leaflet of a host valvular structure. The forming the leaflet opening at 510 is performed with a leaflet perforation tool, such as the leaflet perforation tool 400^{a} or the leaflet perforation tool 400^{b} disclosed herein. The method 500 additionally comprises positioning, at 530, a replacement prosthetic valve in a radially compressed configuration within the leaflet opening and radially expanding, at 540, the replacement prosthetic valve. In some examples, the nosecone 430^{a}, 430^{b} can be positioned to form an opening that extends to the coaptation edge of the leaflet, thereby forming a tear in the leaflet that intersects the coaptation edge.

The leaflet perforation tool used in the performance of the method 500 comprises a catheter (for example, the catheter 402^{a} of the leaflet perforation tool 400^{a} or the catheter 402^{b} of the leaflet perforation tool 400^{b}) and a nosecone coupled to a distal end of the catheter (for example, the nosecone 430^{a} of the leaflet perforation tool 400^{a} or the nosecone 430^{b} of the leaflet perforation tool 400^{b}). The nosecone comprises a main body (for example, the main body 440^{a} or the main body 440^{b}) and at least one blade coupled to the main body (for example, the blade(s) 450^{a} or the blade(s) 450^{b}).

The forming the leaflet opening at 510 comprises inserting, at 516, the nosecone into the leaflet such that the nosecone forms the leaflet opening within the leaflet and such that each blade forms a leaflet slit that is connected to the leaflet opening. In some examples, the nosecone comprises a plurality of blades, and the inserting the nosecone into the leaflet at 516 includes advancing the nosecone through the leaflet such that each blade of the plurality of blades forms a respective leaflet slit. Examples of the leaflet opening and the leaflet slit are shown in FIGS. 13A-13B, discussed above. In particular, FIG. 13A may be described as illustrating a leaflet subsequent to the inserting the nosecone into the leaflet at 516 and forming respective leaflet slits with circumferentially opposed blades of the nosecone.

In some examples, the forming the leaflet opening at 510 comprises forming, at 512, a pilot puncture within the leaflet with a lacerating member of the leaflet perforation tool (such as the lacerating member 410^{a}, the lacerating member 410^{b}, or a guidewire). In such examples, the forming the pilot puncture at 512 is performed prior to the inserting the leaflet perforation tool into the leaflet at 516, which in turn includes inserting the nosecone into the pilot puncture to expand the pilot puncture into the leaflet opening. In some examples, the forming the pilot puncture at 512 comprises translating the lacerating member in a distal direction relative to the nosecone and through the leaflet to form the pilot puncture.

In some examples, the method 500 comprises retracting the lacerating member away from the leaflet subsequent to the forming the pilot puncture at 512 and prior to the inserting the nosecone into the leaflet at 516. In other examples, the inserting the nosecone into the leaflet at 516 may be performed while the lacerating member extends through the leaflet.

In some examples, one or more steps of the method 500 may be performed and/or facilitated with one or more guidewires. For example, the leaflet perforation tool may be advanced toward and/or positioned relative to the leaflet over a perforation tool guidewire, such as the perforation tool guidewire 426^{a} or the perforation tool guidewire 426^{b} disclosed herein. As noted above, the guidewire 426^{a}, 426^{b} can be used as the lacerating member, and/or can be used in lieu or in addition to the lacerating member 410^{a} or 410^{b} in the form of a needle, to form the pilot puncture. As another example, the positioning the replacement prosthetic valve within the leaflet opening at 530 can comprise advancing the replacement prosthetic valve into the leaflet opening via a replacement valve guidewire. In some examples, the replacement valve guidewire and the perforation tool guidewire refer to the same guidewire. In some other examples, the perforation tool guidewire extends alongside the replacement valve guidewire.

The radially expanding the replacement prosthetic valve at 540 may be performed in any suitable manner, such as using any suitable valve expansion technique and/or mechanism that is known to the art. For example, the radially expanding the replacement prosthetic valve at 540 can comprise inflating a valve delivery inflatable balloon on which the replacement prosthetic valve is mounted. As another example, the radially expanding the replacement prosthetic valve at 540 can comprise actuating a mechanical actuator of the replacement prosthetic valve to mechanically expand the frame of the replacement prosthetic valve. In another example, the radially expanding the replacement heart valve can comprise deploying a self-expandable prosthetic valve.

With the replacement prosthetic valve received within the leaflet opening, the radially expanding the replacement prosthetic valve at 540 can operate to increase a size of the leaflet opening and/or to tear the leaflet. As a result, and as discussed above, the radially expanding the replacement prosthetic valve at 540 can operate to modify the leaflet such that the leaflet does not obstruct a frame opening in a frame of the replacement prosthetic valve. For example, radially expanding the replacement prosthetic valve within the leaflet opening can operate to push a portion of the leaflet extending radially exterior of the replacement prosthetic valve below an upper edge of an outer skirt of the replacement prosthetic valve (toward the inflow end of the replacement valve) and/or away from one or more frame openings of the replacement prosthetic valve.

In some examples, the method 500 can comprise repeating one or more steps disclosed above to form a plurality of punctures in the host valvular structure. For example, the forming the leaflet opening at 510 can comprise forming a first leaflet opening in a first leaflet, and the method 500 further can comprise forming, at 520, a second leaflet opening within a second leaflet of the host valvular structure. The forming the second leaflet opening at 520 can comprise forming, at 522, a second pilot puncture within the second leaflet and inserting, at 526, the nosecone into the second leaflet.

In some examples, forming the second leaflet opening can provide further access and/or fluid paths through the frame of the replacement prosthetic valve. For example, radially expanding the replacement prosthetic valve within the first leaflet opening may push the second leaflet against the frame of the replacement prosthetic valve such that the second leaflet opening is aligned with a frame opening of the frame of the replacement prosthetic valve. Thus, the second leaflet opening can provide additional unobstructed paths through the frame of the replacement prosthetic valve. Moreover, in an example in which the host valvular structure is a previously implanted prosthetic valve, expanding the replacement prosthetic valve within the first leaflet opening can trap the second leaflet opening between the respective frames of the previously implanted prosthetic valve and the replacement prosthetic valve, thereby providing additional access and/or flow paths through each of the frames.

Thus, forming the second leaflet opening can ensure that a greater number of frame openings of the frame are uncovered, and/or that a greater proportion of the frame is uncovered, relative to an example in which only one leaflet is punctured to form a leaflet opening. This may be particularly beneficial in examples in which the frame of a previously implanted prosthetic valve extends axially beyond one or both of the coronary arteries when the replacement prosthetic valve is implanted within a native heart valve.

For example, in some patient anatomies, the left coronary artery is positioned lower (that is, proximate to the host valvular structure) than the right coronary artery. In such examples, the right coronary artery may be sufficiently far from the host valvular structure that implanting the replacement prosthetic valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In other examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic valve may ensure the ability for future access into both coronary arteries or perfusion through the frame to both coronary arteries during the diastole phase of the cardiac cycle. In some examples, the host valvular structure can be modified such that the replacement prosthetic valve is implanted a first leaflet that faces the left coronary artery, and such that the second leaflet opening is formed in a second leaflet that faces the right coronary artery (or vice-versa).

The forming the second pilot puncture at 522 may be performed in any of a variety of manners. For example, the forming the second pilot puncture at 522 can comprise translating, at 524, the lacerating member in a distal direction relative to the catheter to pierce the second leaflet to form the second pilot puncture.

In some examples, the forming the first leaflet opening at 510 can be performed prior to the forming the second leaflet opening at 520. In other examples, the forming the second leaflet opening at 520 can be performed prior to the forming the first leaflet opening at 510.

In an example in which the method 500 comprises the forming the first leaflet opening at 510 and the forming the second leaflet opening at 520, the positioning the replacement prosthetic valve at 530 may comprise positioning the replacement prosthetic valve in the first leaflet opening. Accordingly, in such examples, the radially expanding the replacement prosthetic valve at 540 can comprise radially expanding the replacement prosthetic valve within the first leaflet opening. This is not required, however, and it additionally is within the scope of the present disclosure that the replacement prosthetic valve can be positioned within and expanded within the second leaflet opening.

Moreover, in the method 500, the replacement valve need not be implanted within an opening 52 of a leaflet. For example, in cases where the nosecone forms a full tear in a leaflet that extends to the coaptation edge of the leaflet, the replacement prosthetic valve can be positioned at a location between the leaflets of the host valvular structure (such that the delivery device used to implant to replacement prosthetic valve does not extend through the tear) and then expanded. In other examples, although less desirable, in cases where the opening 52 does not form a full tear in the leaflet, the replacement valve the replacement valve can be positioned at a location between the leaflets of the host valvular structure (such that the delivery device used to implant to replacement prosthetic valve does not extend through the opening) and then expanded.

The current specification further discloses exemplary delivery assemblies and apparatuses that can include both a hole-dilation balloon and a valve-expanding balloon. Any delivery apparatus described herein below is advantageously configured to modify the host valvular structure 12 (i.e., modify at least one of the host leaflets 10), and implant a guest prosthetic valve 100 within the modified valvular structure 12, without the need to switch between separate delivery apparatuses for each function.

Any delivery assembly disclosed herein, comprises a delivery apparatus according to any of the examples described below, and a balloon expandable prosthetic valve. While examples of a delivery assembly described in the current disclosure, are shown to include an exemplary delivery apparatus and a balloon expandable prosthetic valve, it should be understood that a delivery apparatus according to any example of the current disclosure can be used for implantation of other prosthetic devices aside from prosthetic valves, such as stents or grafts.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

FIG. 18 illustrates an exemplary delivery assembly 600 that includes an exemplary delivery apparatus 602 adapted to deliver a balloon expandable prosthetic valve 100, such as prosthetic valve 100 described above with respect to FIGS. 2A-2B. According to some examples, the delivery apparatus 602 includes a handle 604 and a balloon catheter 610 having a valve expansion balloon 664 mounted on its distal end. The delivery apparatus 602 has a tandem balloon arrangement, further including a hole dilation balloon 650 distal to the valve expansion balloon 664. The balloon expandable prosthetic valve 100 can be carried in a crimped state over the balloon catheter 610 or over another catheter disposed around the balloon catheter 610. Optionally, an outer delivery shaft 608 can concentrically extend over the balloon catheter 610, and a push shaft 628 can be disposed over the balloon catheter 610, optionally between the balloon catheter 610 and the outer delivery shaft 608.

In some examples, the delivery apparatus 602 can further include a cover shaft 624, concentrically extending over the balloon catheter 610, optionally within the outer delivery shaft 608. In some examples, as illustrated in FIG. 18, the cover shaft 624 is disposed over the balloon catheter 610, such as between the balloon catheter 610 and the push shaft 628, and positioned in a first position such that its distal portion can surround the valve expansion balloon 664 during delivery toward the site of implantation, wherein the prosthetic valve 100 can be carried in a crimped state over the cover shaft 624.

In some examples, the cover shaft 624 is disposed over both balloon catheter 610 and push shaft 628, such as between the push shaft 628 and the push shaft 628 and the outer delivery shaft 608. In some examples, the delivery apparatus 602 does not include a separate cover shaft. In some examples, the outer delivery shaft 608 serves also as a cover shaft.

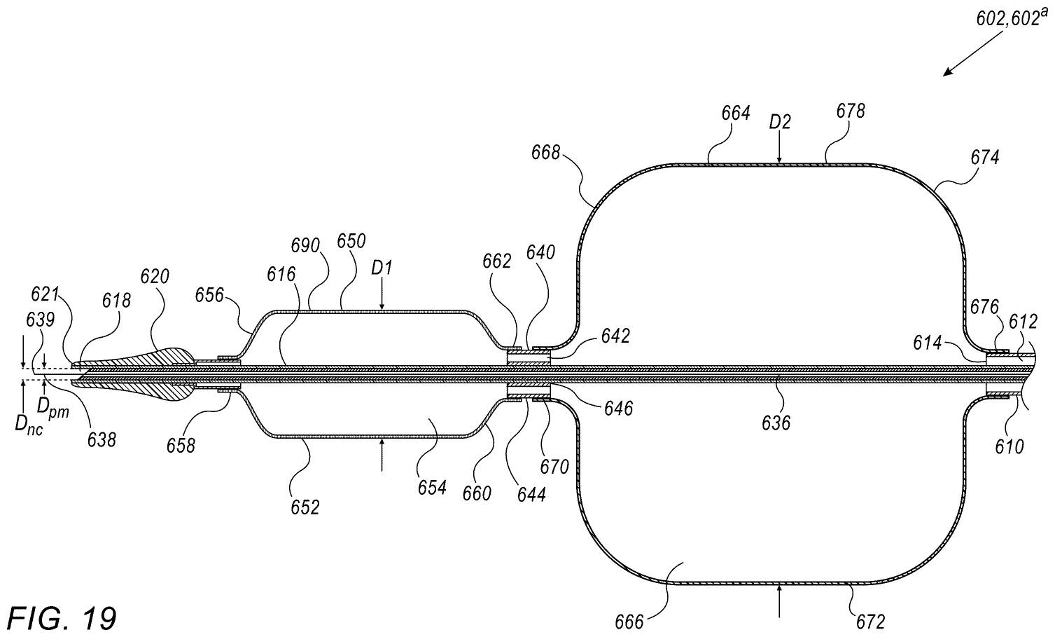

The outer delivery shaft 608, the push shaft 628, the cover shaft 624, and the balloon catheter 610, can be configured to be axially movable relative to each other. For example, a proximally oriented movement of the outer delivery shaft 608 relative to the balloon catheter 610, or a distally oriented movement of the balloon catheter 610 relative to the outer delivery shaft 608, can expose the prosthetic valve 100 from the outer delivery shaft 608. Similarly, a proximally oriented movement of the cover shaft 624 relative to the balloon catheter 610, or a distally oriented movement of the balloon catheter 610 relative to the cover shaft 624, can expose the valve expansion balloon 664 from the cover shaft 624. The delivery apparatus 602 can further include a nosecone 620 carried by a nosecone shaft 616 (hidden from view in FIG. 18, but shown in FIGS. 19-22) extending through a lumen 612 of the balloon catheter 610 (balloon catheter lumen 612 shown in FIGS. 19-22).

The proximal ends of the balloon catheter 610, the outer delivery shaft 608, the push shaft 628, and optionally the nosecone shaft 616 and/or the cover shaft 624, can be coupled to the handle 604. During delivery of the prosthetic valve 100, the handle 604 can be maneuvered by an operator (for example, a clinician or a surgeon) to axially advance or retract components of the delivery apparatus 602, such as the nosecone shaft 616, the balloon catheter 610, the outer delivery shaft 608, the push shaft 628, the cover shaft 624, and/or a perforating member 630 which will described in further detail below, through the patient's vasculature and/or along the target site of implantation, as well as to inflate the hole dilation balloon 650 mounted on the balloon catheter 610, so as to enlarge a leaflet opening 52 as will be elaborated in further detail below, to inflate the valve expansion balloon 664 mounted on the balloon catheter 610, so as to expand the prosthetic valve 100, and to deflate the balloon 650, 664 and retract the delivery apparatus 602 once the prosthetic valve 100 is mounted in the implantation site (for example, within the host valve).

The balloon catheter 610 can extend through the handle 604 and be fluidly connectable to a fluid source for inflating the valve expansion balloon 664 and/or hole dilation balloon 650. The fluid source comprises an inflation fluid. The term “inflation fluid”, as used herein, means a fluid (for example, saline, though other liquids or gas can be used) used for inflating the valve expansion balloon 664 and/or hole dilation balloon 650. The inflation fluid source is in fluid communication with the balloon catheter lumen 612, such as the annular space between the inner surface of balloon catheter 610 and the outer surface of a nosecone shaft 616 disposed therein, such that fluid from the fluid source can flow through the balloon catheter lumen 612, and into any of valve expansion balloon 664 and/or hole dilation balloon 650 to inflate the same. In some examples, as will be further elaborated below, the nosecone shaft 616 can be also fluidly connectable to a fluid source for inflating the hole dilation balloon 650 independently from valve expansion balloon 664.

In some examples, both balloons 664 and 650 can be coupled to balloon catheter 610. In some examples, the valve expansion balloon 664 is coupled to a distal end portion of the balloon catheter 610, while the hole dilation balloon 650 is coupled, directly or indirectly, to another component of the delivery apparatus 602, such as the nosecone shaft 616. In some examples, both balloons 664, 650 are serially arranged over a distal portion of the delivery apparatus, also referred to herein as a tandem balloon arrangement. In some examples, both balloons 664, 650 are separate components that can be attached to each other and/or to other components of the delivery apparatus. In some examples, both balloons 664, 650 are integrally formed, provided as the proximal and distal portions, respectively, of a single unitary balloon, wherein each portion 664, 650 of the unitary balloon is inflatable to a different maximum diameter.

In some examples, both balloons 664, 650 are in fluid communication with each other, such that both can be filled by inflation fluid from a single fluid inflation source, flowing, for example, through the balloon catheter 610. In some examples, the balloons 664, 650 are fluidly sealed from each other, wherein each of the balloons can be filled by inflation fluid flowing through a different shaft. For example, the valve expansion balloon 664 can be in fluid communication with the lumen of the balloon catheter, such that inflation fluid provided into balloon catheter 610 can flow into valve expansion balloon 664, while the hole dilation balloon 650 can be in fluid communication with the lumen 618 of the nosecone shaft, such that inflation fluid provided into nosecone shaft 616 can flow into hole dilation balloon 650. The delivery assembly 600 can further include a guidewire 638 (shown, for example, in FIG. 19) extending through the balloon catheter 610, which refers to any example in which the guidewire 638 can pass through a lumen of any shaft extending through the balloon catheter 610, such as a lumen of a nosecone shaft and/or a lumen of a perforating member, as will be described in further detail below.

The handle 604 can include a steering mechanism configured to adjust the curvature of the distal end portion of the delivery apparatus 602. In the illustrated example, the handle 604 can include an adjustment member, such as the illustrated rotatable knob 606a, which in turn is operatively coupled to the proximal end portion of a pull wire. The pull wire can extend distally from the handle 604 through the outer delivery shaft 608 and has a distal end portion affixed to the outer delivery shaft 608 at or near the distal end of the outer delivery shaft 608. Rotating the knob 606a can increase or decrease the tension in the pull wire, thereby adjusting the curvature of the distal end portion of the delivery apparatus 602. Further details on steering or flex mechanisms for the delivery apparatus can be found in U.S. Pat. No. 9,339,384, which is incorporated by reference herein. The handle 604 can further include an adjustment mechanism including an adjustment member, such as the illustrated rotatable knob 606^{b}. The adjustment mechanism can be configured to adjust the axial position of the push shaft 628 relative to the balloon catheter. The handle can include additional adjustment mechanisms controllable by additional knobs to maneuver additional components of the delivery apparatus 602, such as axial movement of a perforating member 630 and/or axial movement of a push shaft 628, relative to other shafts of the delivery apparatus 602, as will be elaborated in greater detail below.

The prosthetic valve 100 can be carried by the delivery apparatus 602 during delivery in a crimped state, and expanded by inflation of valve expansion balloon 664 to secure it in a native heart valve annulus (such as aortic annulus 24) or against a previously implanted prosthetic valve. In an exemplary implantation procedure, the prosthetic valve 100 can be initially crimped over the balloon catheter 610, proximal to the valve expansion balloon 664. Because prosthetic valve 100 is crimped at a location different from the location of valve expansion balloon 664, prosthetic valve 100 can be crimped to a lower profile than would be possible if it was crimped on top of valve expansion balloon 664. This lower profile permits the clinician to more easily navigate the delivery assembly 600 (including crimped prosthetic valve 100) through a patient's vasculature to the treatment location. The lower profile of the crimped prosthetic valve is particularly helpful when navigating through portions of the patient's vasculature which are particularly narrow, such as the iliac artery. As mentioned above, in some examples, the prosthetic valve 100 can be initially crimped over the cover shaft 624, proximal to the valve expansion balloon 664. During delivery, the distal end portion of the push shaft 628 is positioned proximal to the outflow end 106 of prosthetic valve 100.

When reaching the host valve, the delivery apparatus 602 can be utilized to modify at least one host leaflet 10 as will be described in further detail below, after which the deflated valve expansion balloon 664, carrying crimped prosthetic valve 100 thereover, can be advanced to the target site to expand the prosthetic valve 100. Prior to inflation of valve expansion balloon 664, the push shaft 628 can be advanced distally, allowing its distal end portion to contact and push against the outflow end 106 of prosthetic valve 100, pushing the prosthetic valve 100 distally therewith. The distal end of push shaft 628 is dimensioned to engage with the outflow end 106 of prosthetic valve 100 in a crimped configuration of the valve. In some examples, the distal end portion of the push shaft 628 can be flared radially outward, to terminate at a wider-diameter that can contact the prosthetic valve 100 in its crimped state. Push shaft 628 can then be advanced distally, pushing the prosthetic valve 100 therewith, until the crimped prosthetic valve 100 is disposed around the valve expansion balloon 664, and in some examples, around a distal portion of the cover shaft 624 covering the valve expansion balloon 664, at which point the valve expansion balloon 664 can be inflated, optionally after retraction of the cover shaft 624, to radially expand the prosthetic valve 100. Once the prosthetic valve 100 is expanded to its functional diameter within a native annulus or within a previously implanted host prosthetic valve, the valve expansion balloon 664, as well as the hole dilation balloon 650, can be deflated, and the delivery apparatus 602 can be retrieved from the patient's body.

In some examples, any exemplary delivery assembly of the current disclosure (including delivery assembly 600 and/or delivery assembly 700, described in further detail below) can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

Each of the hole dilation balloon 650 and valve expansion balloon 664 is configured to transition between a deflated state and an inflated state. FIGS. 19-22 show cross-sectional views of a distal portion of some examples of delivery assemblies 600, illustrated in a fully inflated state of both the hole dilation balloon 650 and the valve expansion balloon 664. The hole dilation balloon 650 in these examples is distal to the valve expansion balloon 664. As shown, the hole dilation balloon 650 is smaller in diameter than the valve expansion balloon 664 in their fully inflated states. In some examples, the maximum diameter D1 of the hole dilation balloon 650 in its inflated state is less than 75% of the maximum diameter D2 of the valve expansion balloon 664 in its fully inflated state. In some examples, the maximum diameter D1 of the hole dilation balloon 650 in its inflated state is less than 50% of the maximum diameter D2 of the valve expansion balloon 664 in its fully inflated state. In some examples, the maximum diameter D1 of the hole dilation balloon 650 in its inflated state is less than 40% of the maximum diameter D2 of the valve expansion balloon 664 in its fully inflated state. In some examples, the maximum diameter D1 of the hole dilation balloon 650 in its inflated state is less than 30% of the maximum diameter D2 of the valve expansion balloon 664 in its fully inflated state.

In some examples, the maximum diameter D1 to which the hole dilation balloon 650 can be inflated is equal to or less than 14 mm. In some examples, the maximum diameter D1 is equal to or less than 12 mm. In some examples, the maximum diameter D1 is equal to or less than 10 mm. In some examples, the maximum diameter D1 is equal to or less than 8 mm. In some examples, the maximum diameter D2 to which the valve expansion balloon 664 can be inflated is at least 23 mm. In some examples, the maximum diameter D2 is at least 30 mm. In some examples, the maximum diameter D2 is at least 25 mm. In some examples, the maximum diameter D2 is at least 30 mm. In some examples, the maximum diameter D2 is at least 40 mm.

The hole dilation balloon 650 can comprise a dilation balloon distal tapering segment 656 configured to assume a tapering configuration in the inflated state of the balloon 650, extending from a narrower diameter at a distal end thereof to a wider diameter at its proximal end, and a dilation balloon main segment 690 extending proximally from the dilation balloon distal tapering segment 656. In some examples, the dilation balloon main segment 690 can have a uniform diameter along its length in the inflated state. In some examples, as shown in FIGS. 19-21, the hole dilation balloon 650 can further comprise a dilation balloon proximal tapering segment 660 tapering from a larger diameter of the dilation balloon main segment 690 to a smaller diameter at the proximal end of dilation balloon proximal tapering segment 660.

The valve expansion balloon 664 can comprise an expansion balloon proximal tapering segment 674 configured to assume a tapering configuration in the inflated state of the balloon 664, extending from a narrower diameter at a proximal end thereof to a wider diameter at its distal end, and an expansion balloon main segment 678 extending distally from the expansion balloon proximal tapering segment 674. In some examples, the expansion balloon main segment 678 can have a uniform diameter along its length in the inflated state. In some examples, as shown in FIGS. 19-21, the valve expansion balloon 664 can further comprise an expansion balloon distal tapering segment 668 tapering from a larger diameter of the expansion balloon main segment 678 to a smaller diameter at the distal end of expansion balloon distal tapering segment 668.

Dilation balloon distal tapering segment 656 can include a distal attachment segment 658 that can be secured to a component of the delivery apparatus 602, such as a distal portion of balloon catheter 610, a distal portion of the nosecone shaft 616, a proximal portion of nosecone 620, or to components coupled to nosecone 620. Expansion balloon proximal tapering segment 674 can include an expansion balloon proximal attachment segment 676 that can be secured to a component of the delivery apparatus 602, such as balloon catheter 610. In some examples, as shown in FIGS. 19-21, dilation balloon proximal tapering segment 660 can include a dilation balloon proximal attachment segment 662, and expansion balloon distal tapering segment 668 can include an expansion balloon distal attachment segment 670, wherein each of the attachment segments 662, 670 can be secured to a component of the delivery apparatus 602, such as balloon catheter 610, a component attached to balloon catheter 610 or nosecone shaft 616, or to each other. Any of the attachment segments 658, 662, 670, 676 can be secured to another component of the delivery apparatus 602 by force fitting, heat pressing, welding or a suitable adhesive.

As further shown in FIGS. 19-22, the nosecone shaft 616 extends through the balloon catheter lumen 612, and defines a nosecone shaft lumen 618 extending along the entire length of the nosecone shaft 616 and nosecone 620, through which a guidewire 638 can pass, such that the entire delivery apparatus 602 can be advanced toward the treatment region over the guidewire 638. In some examples, a perforating member 630 can extend through the balloon catheter 610, such as by extending through balloon catheter lumen 612 and/or a lumen of a shaft extending through balloon catheter 610, such as through a nosecone shaft lumen 618 of a nosecone shaft 616. In some examples, the perforating member 630 is axially movable relative to another component of the delivery apparatus 602, such as the hole dilation balloon 650. In some examples, a perforating member 630 can extend through nosecone shaft lumen 618, and be axially movable relative to nosecone shaft 616. The perforating member 630 can define a perforating member lumen 636, through which the guidewire 638 can extend.

Nosecone shaft 616, balloon catheter 610, cover shaft 624 and optional outer delivery shaft 608 can be formed from any of various suitable materials, such as nylon, braided stainless steel wires, or a polyether block amide (commercially available as Pebax®). In some examples, balloon catheter 610, cover shaft 624 and optional outer delivery shaft 608 have longitudinal sections formed from different materials in order to vary the flexibility of the shafts along their lengths. In some examples, nosecone shaft 616 has an inner liner or layer formed of Teflon® to minimize sliding friction with a guidewire 638 and/or a perforating member 630.

Nosecone shaft 616 defines an inner diameter D_{nc}, which is the diameter of nosecone shaft lumen 618. Perforating member 630 defines an outer diameter Dₚₘ, and may be implemented as a hollow needle, through which a guidewire 638 having a guidewire diameter D_{gw} can extend. While many conventional nosecone shafts are designed to have an inner diameter that merely allows a guidewire 638 to pass therethrough, the nosecone 620 may have a relatively larger inner diameter D_{nc} to allow both the perforating member 630 and the guidewire 638 to extending therethrough and be axially movable within the nosecone shaft lumen 618. Thus, a perforating member 630 can have perforating member lumen 636 dimensioned similarly to conventional nosecone shaft lumens, to allow for similar axial movement of a guidewire 638 therein, necessitating the inner diameter D_{nc} of non-conventional nosecone shaft 616 to be, in turn, greater in size, and specifically, greater than the outer diameter Dₚₘ of the perforating member 630.

Hole dilation balloon 650 comprises a dilation balloon wall 652 having a wall thickness T1 and defining an internal cavity 654. Valve expansion balloon 664 comprises an expansion balloon wall 672 having a wall thickness T2 and defining an internal cavity 666. In some examples, the dilation balloon cavity 654 is in fluid communication with the expansion balloon cavity 666, such that inflation fluid may freely flow between both cavities 654, 666.

The outer diameter of nosecone shaft 616 can be sized such that an annular space is formed within balloon catheter lumen 612 between balloon catheter 610 and nosecone shaft 616 along the length of balloon catheter 610. This annular space can be in fluid communication with one or more balloon catheter openings 614 exposed to an internal cavity 666 of the valve expansion balloon 664, which can be in fluid communication with a fluid source (for example, a syringe or a pump) that can inject an inflation fluid (for example, saline) into expansion balloon cavity 666. In this way, fluid from the fluid source can flow through balloon catheter lumen 612, and into expansion balloon cavity 666 via balloon catheter opening(s) 614, which serves to inflate the valve expansion balloon 664 and expand and deploy a prosthetic valve 100 disposed thereon, if inflation of valve expansion balloon 664 is not restricted by a cover shaft 624 or other restriction means, as will be described in further detail below.

When cavities 654, 666 are in fluid communication, inflation fluid flowing into expansion balloon cavity 666 can flow therefrom into dilation balloon cavity 654, which can in turn inflate the hole dilation balloon 650, even when inflation of valve expansion balloon 664 is restricted. The pressure of the inflation fluid within hole dilation balloon 650 may provide the force that allows the main segment 690 of hole dilation balloon 650 to dilate a leaflet opening 52 in a manner that will be further described below. The pressure of the inflation fluid within valve expansion balloon 664, when valve expansion balloon 664 is not restricted from expanding, may provide the force that allows the main segment 678 of valve expansion balloon 664 to expand a prosthetic valve 100 disposed thereon. Further, the balloon catheter lumen 612 may be configured to withdraw fluid from expansion balloon cavity 666 and/or dilation balloon cavity 654 through the balloon catheter opening(s) 614, to deflate the valve expansion balloon 664 and/or hole dilation balloon 650, respectively.

While balloon catheter 610 is shown to terminate at a proximal end of valve expansion balloon 664 throughout FIGS. 19-22, in some examples, balloon catheter 610 can extend farther in the distal direction, through a portion or through the entire length of expansion balloon cavity 666 and/or dilation balloon cavity 654, and one or more balloon catheter opening(s) 614 can be formed on the sidewall of balloon catheter 610, exposed laterally to expansion balloon cavity 666 and/or dilation balloon cavity 654.

Various exemplary implementations for delivery assemblies 600 can be referred to, throughout the specification, with superscripts, for ease of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, including delivery apparatus 602, referred to with a superscript, may be optionally shared by some but not necessarily all other exemplary implementations. For example, delivery apparatus 602^{a} is an exemplary implementation of delivery apparatus 602, and thus includes all of the features described for delivery apparatus 602 throughout the current disclosure, except that while a delivery apparatus 602 can be generally provided with or without an intermediate connector disposed between balloons 650 and 664, delivery apparatus 602^{a} does include an intermediate connector 640 provided with an intermediate channel 642 that facilitates fluid communication between cavities 654 and 666, respectively, as will be described in further detail below.

FIG. 19 shows a cross sectional view of an exemplary delivery apparatus 602^{a}. As mentioned above, delivery apparatus 602^{a} is an exemplary implementation of delivery apparatus 602, and thus includes all of the features described for delivery apparatus 602 throughout the current disclosure, except that while delivery apparatus 602 can be provided with or without an intermediate connector, delivery apparatus 602^{a} comprises an intermediate connector 640 which can be a tubular or ring-like member to disposed between the balloons 650, 664 and to which both balloons 650, 664 can be attached. The intermediate connecter 640 can be disposed around a shaft of the delivery apparatus 602^{a}. In the illustrated example, the intermediate connector 640 defines a connector inner surface 646 that can be coupled to (for example, by gluing, welding, and the like) an outer surface the nosecone shaft 616. In examples in which the balloon catheter extends through the cavities 654, 666, the connector inner surface 646 that can be coupled to an outer surface of the balloon catheter 610. Any of the dilation balloon proximal attachment segment 662 and the expansion balloon distal attachment segment 670 can be similarly coupled to an outer surface 644 of the intermediate connector 640. The intermediate connector can have a diameter, defined by the connector outer surface 644, which is smaller than the maximum diameter of the hole dilation balloon 650 in its inflated state, such that the intermediate connector 640 forms a narrow neck portion between both balloons 650, 664.

As shown, the intermediate connector 640 further defines at least one intermediate channel 642 extending across its length, between the connector outer surface 644 and the connector inner surface 646, facilitating fluid communication between the dilation balloon cavity 654 and expansion balloon cavity 666.

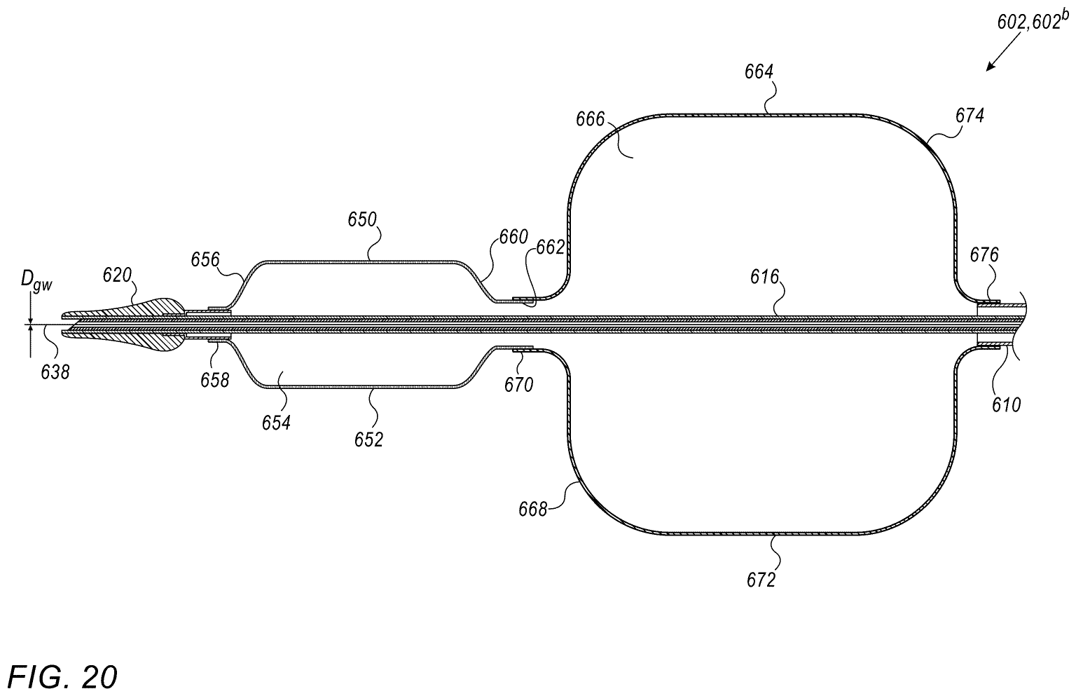

FIG. 20 shows a cross sectional view of an exemplary delivery apparatus 602^{b}. Delivery apparatus 602^{b} is an exemplary implementation of delivery apparatus 602, and thus includes all of the features described for delivery apparatus 602 throughout the current disclosure, except that the dilation balloon proximal attachment segment 662 and the expansion balloon distal attachment segment 670 of delivery apparatus 602^{b} are coupled to each other, optionally overlaying over one another and glued or welded to each other. In contrast to delivery apparatus 602^{a} described above with respect to FIG. 19, delivery apparatus 602^{b} does not include an intermediate connector, and the dilation balloon proximal attachment segment 662 and the expansion balloon distal attachment segment 670 are radially spaced from the inner shaft, which is the nosecone shaft 616 in the illustrated example, or a balloon catheter 610 in other example, at least in the inflated states of balloon 650 and/or balloon 664, such that the space formed between the dilation balloon proximal attachment segment 662 and the expansion balloon distal attachment segment 670 and the inner shaft (for example, nosecone shaft 616) facilitates fluid communication between the dilation balloon cavity 654 and expansion balloon cavity 666.

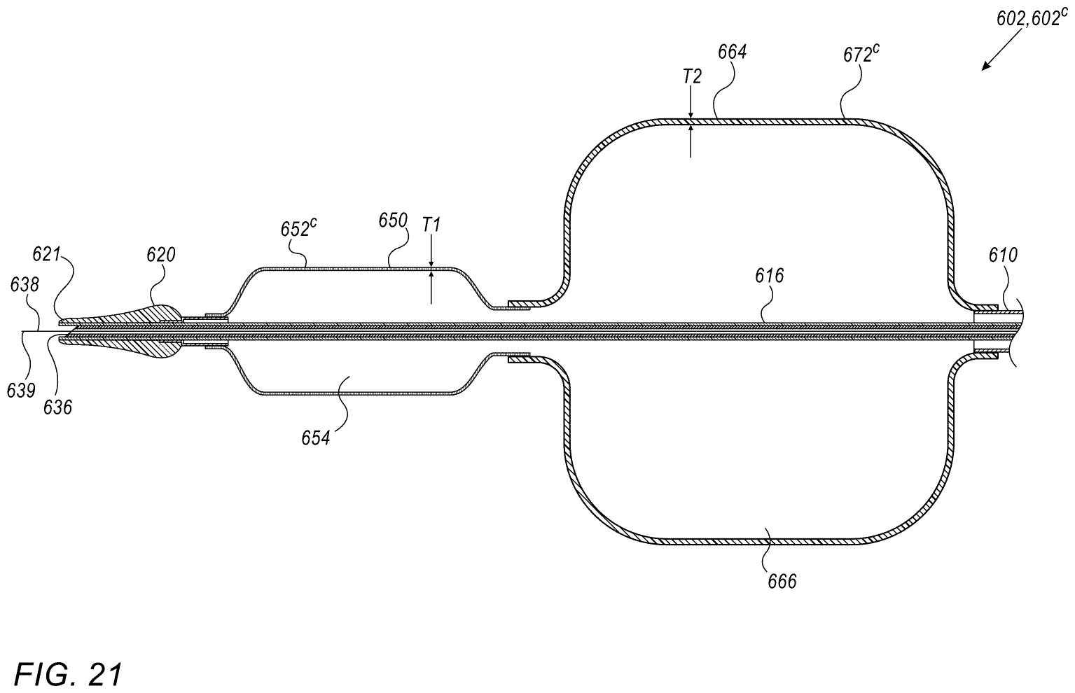

FIG. 21 shows a cross sectional view of an exemplary delivery apparatus 602^{c}. Delivery apparatus 602^{c} is an exemplary implementation of delivery apparatus 602, and thus includes all of the features described for delivery apparatus 602 throughout the current disclosure, except that while the wall thicknesses T1 and T2 of dilation balloon wall 652 and expansion balloon wall 672, respectively, can assume any value for any delivery apparatus 602 including being identical to each other, the wall thicknesses T1 of dilation balloon wall 652^{c} is thinner than the wall thicknesses T2 of expansion balloon wall 672^{c} in the case of delivery apparatus 602^{c}. While illustrated without an intermediate connector in FIG. 21, similar to the configuration of delivery apparatus 602^{b} illustrated in FIG. 20, it is to be understood that delivery apparatus 602^{c} can also include, in some examples, an intermediate connector 640, in the same manner described for delivery apparatus 602^{a} with respect to FIG. 19.

Delivery apparatus 602^{c} can be provided, in some examples, without a cover shaft 624, even though the cavities 654, 666 can be in fluid communication with each other. In use, delivering inflation fluid at a pressure that does not exceed a predefined threshold may serve to inflate the more compliant hole dilation balloon 650 while the valve expansion balloon 664 can remain relatively deflated due to the higher thickness T2 of expansion balloon wall 672. Thus, in such cases, the thicker expansion balloon wall 672 can serve as an inflation restraining mechanism in lieu of a cover shaft 624. When inflation of valve expansion balloon 664 is desired, inflation fluid may be provided at a greater pressure, sufficient to inflate the expansion balloon wall 672. While the thinner dilation balloon wall 652 allows inflation of hole dilation balloon 650 at a lower pressure, it is configured to withstand the higher pressure supplied for inflating the valve expansion balloon 664 without tearing or bursting.

In some examples, instead or in addition to having different thicknesses, the balloons 650, 664 can be formed of different materials, such that the material from which the expansion balloon wall 672 is made is more tear resistant than the material from which the dilation balloon wall 652 is made. For example, the material properties of the dilation balloon wall 652 can be more compliant such that hole dilation balloon 650 can expand at pressures which are less than a predefined pressure threshold, while the less compliant valve expansion balloon 664 will remain deflated. Thus, in such cases, the less compliant material properties of expansion balloon wall 672 can serve as an inflation restraining mechanism in lieu of a cover shaft 624. When inflation of valve expansion balloon 664 is desired, inflation fluid may be provided at a greater pressure, sufficient to inflate the expansion balloon wall 672. While the more compliant dilation balloon wall 652 allows inflation of hole dilation balloon 650 at a lower pressure, it is configured to withstand the higher pressure supplied for inflating the valve expansion balloon 664 without tearing or bursting. In such examples, the wall thicknesses T1 and T2 can be substantially equal to each other, though non-equal wall thicknesses can be also combined with differing material properties. It is to be understood that any reference throughout the disclosure to examples of thinner hole dilation balloons 650, may similarly refer to hole dilation balloons 650 made of more compliant materials, in addition to, or instead of, being thinner.

The hole dilation balloon 650 and the valve expansion balloon 664 can be formed as two separate balloons, coupled to each other and/or to other components of the delivery apparatus 602, or can be two portions of a unitary balloon.

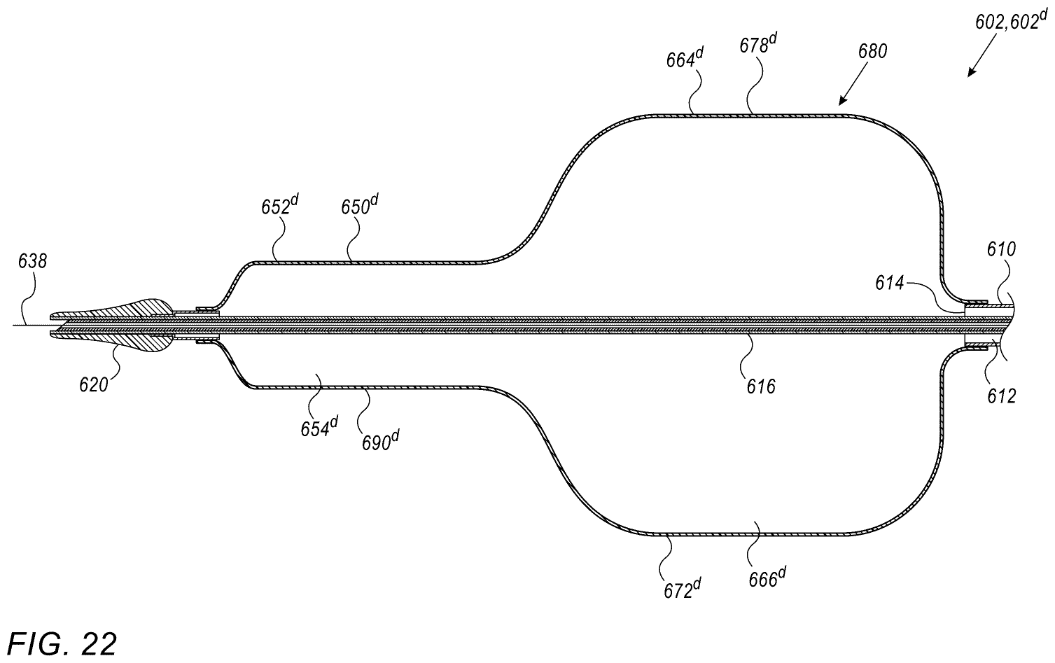

FIG. 22 shows a cross sectional view of an exemplary delivery apparatus 6024. Delivery apparatus 602^{d} is an exemplary implementation of delivery apparatus 602, and thus includes all of the features described for delivery apparatus 602 throughout the current disclosure, except that the delivery apparatus 602^{d} comprises a non-uniform unitary balloon 680, in which case the hole dilation balloon 650^{d} is formed as a distal portion of the unitary balloon 680, and the valve expansion balloon 664^{d} is formed as a proximal portion of the unitary balloon 680, both portions forming a continuous single unitary balloon 680 with the distal portion being smaller in diameter than the proximal portion when fully inflated, as described above with respect to the diameters of balloons 650 and 664. In such cases, the dilation balloon cavity 654^{d} and expansion balloon cavity 666^{d} can together form a single continuous internal cavity of the unitary balloon 680.

In some examples, as illustrated in FIG. 22, the hole dilation balloon 650^{d}, which is the distal portion of unitary balloon 680, does not necessarily include a dilation balloon proximal tapering segment, but rather extends from the diameter of the dilation balloon main segment 690^{d} to the greater diameter of the expansion balloon main segment 678^{d} when fully inflated. Nevertheless, in some examples, the unitary balloon can be shaped to form a narrower neck portion between both portions, similar to the configuration illustrated in FIG. 20, except for the dilation balloon wall 652^{d} and the expansion balloon wall 672^{a} formed as a continuous single wall of the unitary balloon 680.

While illustrated in FIG. 22 to have a uniform wall thickness for the dilation balloon wall 652^{d} and the expansion balloon wall 672^{a} of both portion of the unitary balloon 680, it is to be understood that the unitary balloon 680 can be formed, in some examples, to include a non-uniform wall thickness, such that the wall thickness T1 of the dilation balloon wall 652 along the distal portion of the unitary balloon 680 is thinner than the wall thickness T2 of the expansion balloon wall 672 along the proximal portion of the unitary balloon 680, allowing it to be utilized, in such example, to inflate the distal portion at lower pressures, and increase inflation pressure to inflate the proximal portion as well when desired, in a manner similar to that described above for delivery apparatus 602^{c} with respect to FIG. 21.

FIGS. 23A-23D illustrate some steps in a method for utilizing a delivery assembly 600 for forming a leaflet hole inside a host leaflet, which can be performed prior to implanting a guest prosthetic valve inside the host valvular structure. The delivery assembly 600 can be used to perforate, cut, and/or tear a host leaflet 10, such as a native leaflet 30 or a prosthetic valve leaflet 114 of a previously implanted prosthetic valve. While delivery assembly 600^{a} comprising delivery apparatus 602^{a} is illustrated throughout FIGS. 23A-23D, it is to be understood that other examples of delivery apparatus 602 described in the current specification can be used in a similar manner, as will be further elaborated below.

The distal end portion of the delivery apparatus 602, including nosecone 620, is configured to be inserted into a patient's vasculature, such as within an ascending aorta, and to be advanced towards the host leaflet 10, wherein the guidewire can optionally pierce through the host leaflet 10 as shown in FIG. 23A. Positioning the delivery apparatus 602 relative to the host leaflet 10 may comprise advancing the delivery apparatus 602 toward the leaflet via guidewire 638. As mentioned above, the delivery apparatus 602 can further include a perforating member 630, which can be also referred to as a lacerating member 630. Perforating member 630 comprises a distal end portion 632 configured to pierce a host leaflet 10 of a host valvular structure 12 to form a pilot puncture 50 in the host leaflet 10, when a distal end portion 632 is positioned distal to the hole dilation balloon 650.

In some examples, the distal end portion 632 of perforating member 630 is configured to be selectively translated in the proximal or distal directions relative to another component of the delivery apparatus 602, such as the hole dilation balloon 650 and/or the nosecone 620. In some examples, the nosecone shaft 616 and the perforating member 630 are configured to be movable axially relative to each other in the proximal and distal directions. The perforating member 630 can be coupled to a handle 604. The handle 604 can have one or more actuators (for example, in the form of rotatable knobs 606) that are operatively coupled to the perforating member 630 to facilitate axial movement thereof. In such examples, the distal end portion 632 can be configured to pierce a host leaflet 10 when axially translated to a position which is distal to the hole dilation balloon 650 and/or the nosecone 620. In some examples, the distal end portion 632 is not necessarily configured to be axially translatable relative to the hole dilation balloon 650, in which case it is positioned distal to the hole dilation balloon 650 at all times.

As shown in FIG. 23B, the perforating member 630 is configured to puncture the host leaflet 10 to form a pilot puncture 50 within host leaflet 10. Subsequent to forming the pilot puncture 50, and as shown in FIG. 23C, the hole dilation balloon 650 may be inserted within the pilot puncture 50. The distal end portion 632 of the perforating member 630 can be concealed within a lumen of the delivery apparatus 602, being positioned proximal to a distal end 621 of the nosecone 620 during delivery, as shown in FIG. 23A, to avoid damage that may be caused to internal anatomical structures of the patient's body due to accidental contact with an optionally sharp distal end portion 632. The distal end portion 632 can be then pushed toward and through the host leaflet 10 to form the pilot puncture 50 as shown in FIG. 23B, after which it can be retracted back to re-conceal the distal end portion 632.

In some examples, the perforating member 630 can be retracted so as to position the distal end portion 632 proximal to the nosecone distal end 621 after forming the pilot puncture 50 and prior to advancing the nosecone 620 to place hole dilation balloon 650 inside the pilot puncture 50, relying on the nosecone distal end 621 being small enough to allow insertion of the tapering nosecone 620 through pilot puncture 50. In some examples, the distal end portion 632 of the perforating member 630 remains distal to the nosecone distal end 621 during advancement of the nosecone 620 and hole dilation balloon 650 through the pilot puncture 50, after which it is retracted to conceal the perforating member 630, positioning its distal end portion 632 proximal to the nosecone distal end 621, as illustrated in FIG. 23C.

With the hole dilation balloon 650 received within the pilot puncture 50, inflating the hole dilation balloon 650 to transition it from the radially deflated state (FIG. 23C) to the radially inflated state (FIG. 23D) can expand the pilot puncture 50 to form a leaflet opening 52 that is sized to receive the prosthetic valve 100 in the radially compressed or crimped configuration.

In some examples, inflating the hole dilation balloon 650 within the host leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In other examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the hole dilation balloon 650 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

The delivery apparatus 602 may be configured to form the leaflet opening 52 in any of a variety of host valvular structures 12. In the example of FIGS. 23A-23D, the host valvular structure 12 can be the valvular structure 113 of a previously implanted prosthetic valve, such as the prosthetic valve 100a of FIG. 3. In such examples, using the delivery assembly 600 as described herein to form the leaflet opening 52 in a previously implanted prosthetic valve may be followed by steps for implanting a guest prosthetic valve 100^{b} within the previously implanted prosthetic valve 100a (for example, via a ViV procedure).

Similarly, the host valvular structure 12 in the example of FIGS. 23A-23D can be a valvular structure 29 of a native heart valve, such as the native aortic valve 20 shown in FIGS. 2A-2B. In such examples, the perforating member 630 can be configured to puncture a native leaflet 30 of the native aortic valve 20. In other examples, the host valvular structure and/or the native valve may refer to another valve of a patient's heart, such as a mitral valve, a pulmonary valve, or a tricuspid valve.

In some examples, the perforating member 630 may include and/or be a needle, such as a spring-loaded needle and/or a Veress needle. As shown in FIGS. 23A-23D, the distal end portion 632 of the perforating member 630 can terminate at an angled surface 634. The angled surface 634 can have a sharp cutting edge to facilitate piercing the host leaflet 10 when the needle is pressed against the leaflet.

Delivery apparatus 602 may include any of a variety of features to facilitate positioning the nosecone 620 and/or the perforating member 630 relative to the host leaflet 10. For example, the nosecone shaft 616, balloon catheter 610, outer delivery shaft 608, and/or the perforating member 630, may be pre-formed, shaped, and/or curved so as to be directed and/or angled toward the host leaflet 10 when positioned in the vicinity of the host valvular structure. Furthermore, one or more shafts of delivery apparatus 602, such as outer delivery shaft 608, can have a steering mechanism (for example, a pull wire and a corresponding adjustment mechanism in the handle 604) to steer or adjust its distal end.

As further mentioned above, in some examples, the perforating member 630 can comprise a perforating member lumen 636, configured to accommodate a guidewire 638 that can extend through the perforating member lumen 636. In such examples, the guidewire 638 can be inserted into the patient's vasculature, and then the perforating member 630 and/or other shafts of the delivery apparatus 602 may be advanced toward the host leaflet 10 over the guidewire 638.

In some examples, the guidewire 638 can be used as a perforating or lacerating member for forming a pilot puncture 50. In such examples, the guidewire 638 can be a relatively stiff wire having a distal tip 639 configured to pierce the host leaflet 10 when the guidewire 638 is pressed against the leaflet. In some examples, the guidewire 638 can include a radio-frequency (RF) energy delivery tip 639 (see, for example, FIG. 19) to assist with penetration through the leaflet tissue. For this purpose, a suitable RF energy device may be coupled to the guidewire 638, and the RF energy device can apply the RF energy to the guidewire tip 639 to penetrate the host leaflet 10. In any examples disclosed herein wherein a guidewire is used to puncture a leaflet, the guidewire can be coupled to a source of RF energy that applies RF energy to the tip of the guidewire. When the guidewire 638 is used to pierce the leaflet 10, the perforating member 630 in the form of a needle can be omitted, or it can be used in combination with the guidewire 638 that forms an initial puncture in the leaflet 10. For example, the guidewire 638 can be used to form an initial pilot puncture 50 (see FIG. 23A), after which the perforating member 630 can be advanced through the leaflet to form a slightly larger pilot puncture (see FIG. 23B) for subsequent advancement of the hole dilation balloon 650 through the host leaflet 10.

In some examples, the guidewire 638 is used as a perforating member without any additional separate perforating member, such as a needle, disposed thereover, such that the guidewire 638 can be utilized as the sole component that forms the pilot puncture 50, allowing other components of the delivery apparatus, such as nosecone 620, to pass through the pilot puncture 50, in a manner similar to that illustrated in FIG. 23B, but without the needle 630.

In some examples, the guidewire 638 is used as a perforating member that can be used in addition to perforating member (for example, needle) 630, such that the guidewire 638 can form an initial puncture via a sharp tip 639 or an RF energy delivery tip 639, as illustrated in FIG. 23A, followed by penetration of the perforating member 630 into the leaflet 10 to form the pilot puncture 50, or a pilot puncture 50 which is greater in size than an initial puncture formed by the guidewire tip 639, as shown in FIG. 23B.

In some examples, the guidewire tip 639 is not necessarily sharp enough or otherwise configured to puncture through the host leaflet 10, in which case the guidewire 638 can be utilized for advancement of the delivery apparatus 602 toward the valvular structure 12, but terminate in proximity of the host leaflet 10 without piercing through it (for example, remaining above host leaflet 10 instead of passing through the tissue as shown in FIG. 23A), and the distal end portion 632 of perforating member 630 can be then advanced toward and into the host leaflet 10, to form the pilot puncture 50 in a similar manner to that illustrated in FIG. 23B.

FIGS. 24A-24B illustrate the hole dilation balloon 650 utilized to expand the pilot puncture 50 into the leaflet opening 52. In particular, FIG. 24A illustrates the hole dilation balloon 650 in the deflated state within the pilot puncture 50, corresponding to the state described above with respect to FIG. 23C, while FIG. 24B illustrates the hole dilation balloon 650 in the inflated state such that the pilot puncture 50 has enlarged into the leaflet opening 52, corresponding to the state described above with respect to FIG. 23D.

As shown throughout FIGS. 23A-24B, the valve expansion balloon 664, positioned proximal to the hole dilation balloon 650, remains in a deflated state throughout these steps of the procedure, including during inflation of hole dilation balloon 650 shown in FIGS. 23D and 24B. In some examples, as shown in FIGS. 23A-24B, a distal portion of a cover shaft 624 is disposed, in the first position, around the valve expansion balloon 664 but not around the hole dilation balloon 650 when the hole dilation balloon 650 is advanced into the pilot puncture 50 of the host leaflet 10 as shown in FIGS. 9C and 10A. In such examples, the prosthetic valve 100 can be optionally crimped over the cover shaft 624, proximal to the valve expansion balloon 664.

When inflation fluid flows into expansion balloon cavity 666 through balloon catheter opening 614, the cover shaft 624, in its first position, prevents the valve expansion balloon 664 from inflating. Since both cavities 654 and 666 are in fluid communication, the inflation fluid flows from the expansion balloon cavity 666 into dilation balloon cavity 654, causing it to inflate and from leaflet opening 52, as shown in FIGS. 23D and 24B.

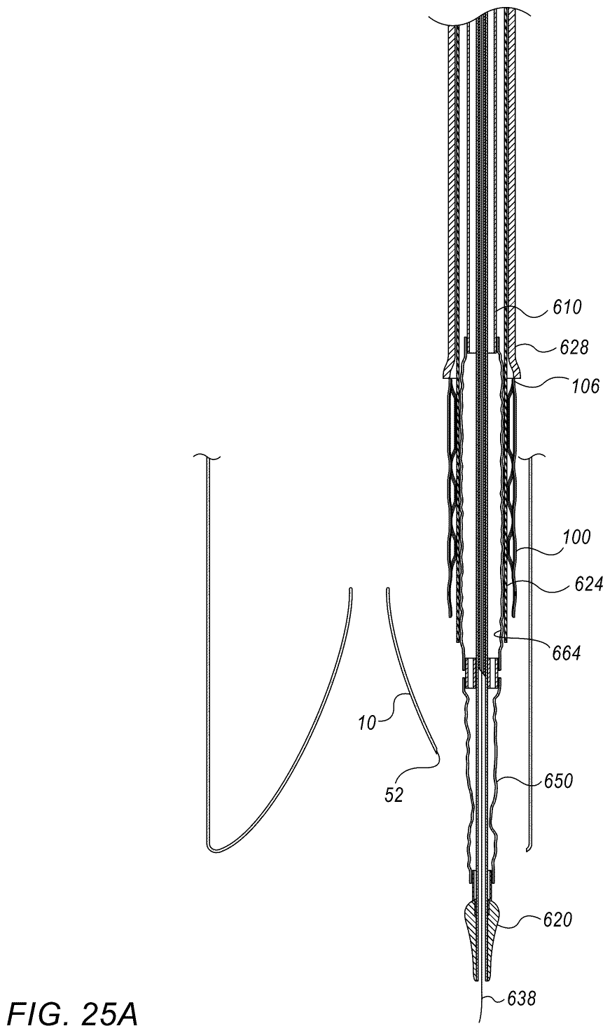

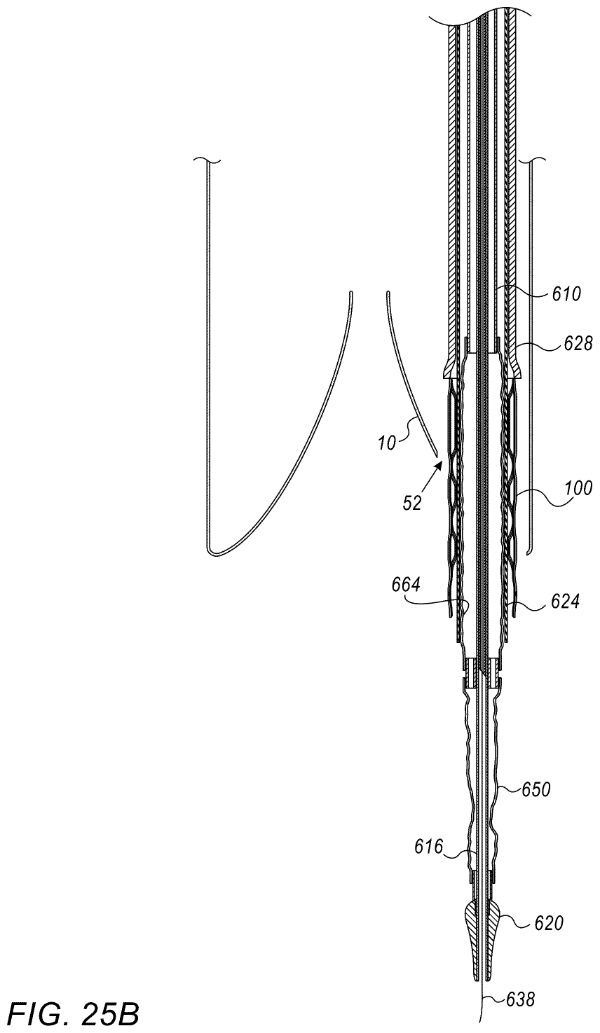

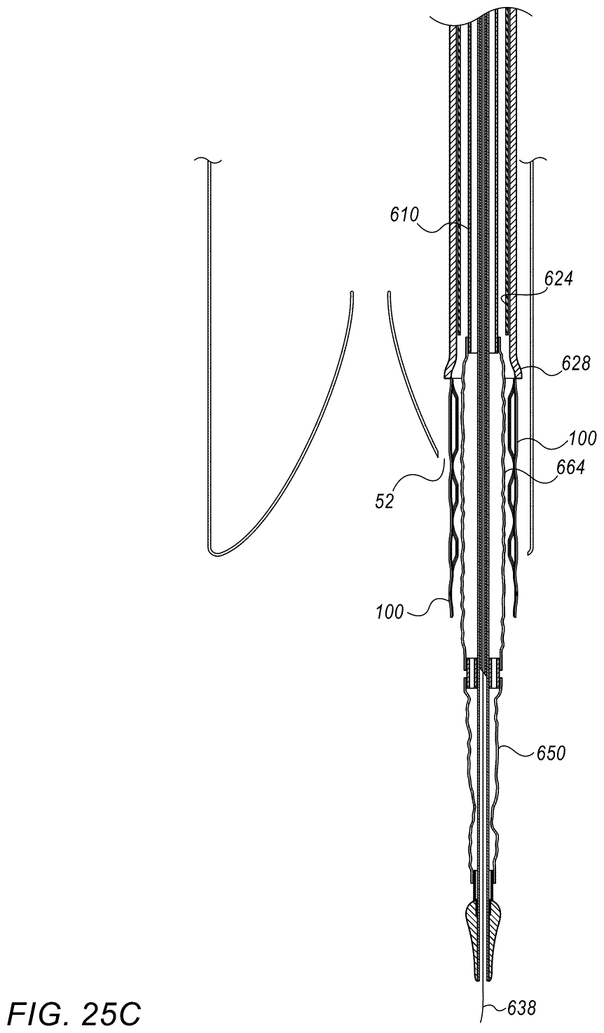

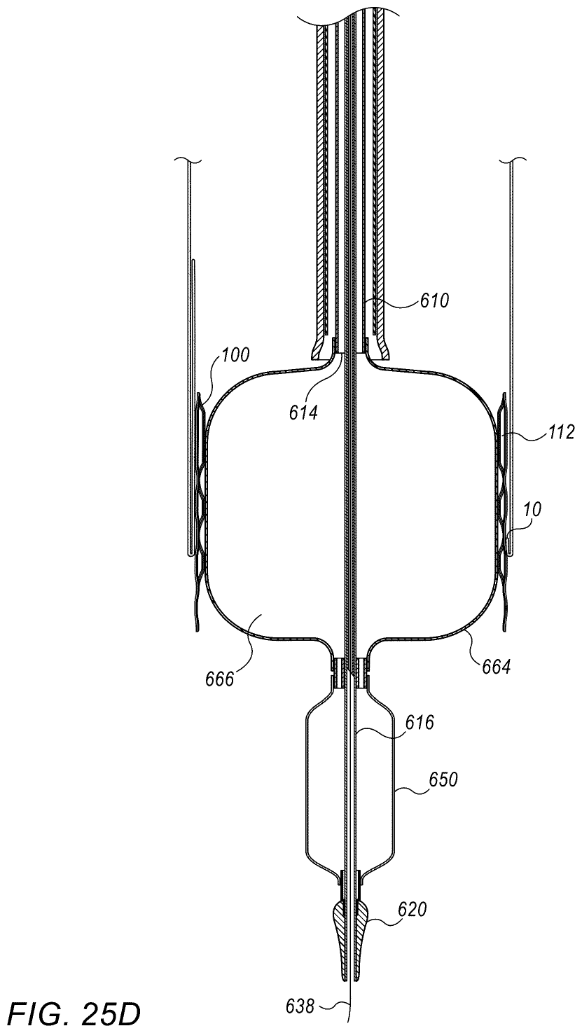

FIGS. 25A-25D show subsequent steps of a method utilizing delivery assembly 600, following the steps described above and illustrated in FIGS. 23A-23D. After the hole dilation balloon 650 is inflated to form the leaflet opening 52 as shown in FIG. 23D, the hole dilation balloon 650 is deflated, as shown in FIG. 25A, and the push shaft 628 can be utilized to distally advance the crimped prosthetic valve 100 toward and around valve expansion balloon 664, such that the cover shaft 624 is disposed between the prosthetic valve 100 and the valve expansion balloon 664.

The deflated valve expansion balloon 664 and the prosthetic valve 100 disposed thereover can be then advanced and positioned within the leaflet opening 52, as shown in FIG. 25B, and the cover shaft 624 can be retracted to a second position so that it doesn't cover the valve expansion balloon 664 any longer, as shown in FIG. 25C. The push shaft 628 can remain in position, abutting outflow end 106 of prosthetic valve 100 during cover shaft 624 retraction, to provide a counter force that resists proximal displacement of the prosthetic valve 100 during cover shaft 624 retraction.

At this stage, delivering inflation fluid into the expansion balloon cavity 666 allows the valve expansion balloon 664 to inflate and expand the prosthetic valve 100, as shown in FIG. 25D. As described in more detail below, expanding the guest prosthetic valve 100 to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame 102 of the guest prosthetic valve 100.

While the prosthetic valve 100 is shown in FIG. 25A to be pushed distally to the position of valve expansion balloon 664 after forming the leaflet opening 52, it is to be understood that the prosthetic valve 100 can be pushed by push shaft 628 over valve expansion balloon 664 at any other stage prior to advancement of the valve expansion balloon 664 into the leaflet opening 52 as shown in FIG. 25B. For example, the prosthetic valve 100 can be pushed over valve expansion balloon 664 upon approximation to the site of implantation, such as upon reaching the region of the ascending aorta 26 even prior to forming the pilot puncture of any of the stages shown in FIGS. 23A-23B. Similarly, the prosthetic valve can be pushed distally after the pilot puncture 50 is formed but before advancing the deflated hole dilation balloon 650 into the pilot puncture 50, or after positioning the hole dilation balloon 650 inside the pilot puncture 50 but before inflating it to form the leaflet opening 52, or after forming the leaflet opening 52 but before deflating the hole dilation balloon 650, or after deflating the hole dilation balloon 650. Similarly, it is to be understood that in some examples, the delivery assembly can be provided without a push shaft 628, and/or that the prosthetic valve 100 can be crimped around the valve expansion balloon 664, optionally over the cover shaft 624, and delivered through the patient's vasculature in this position.

While cover shaft 624 retraction is shown in FIG. 25C after positioning of the prosthetic valve 100 in the leaflet opening 52, it is to be understood that cover shaft 624 can be retracted to the second position at any other stage after deflation of hole dilation balloon 650 as shown in FIG. 25A, including prior to advancement of the prosthetic valve 100 into leaflet opening 52, in which case, the prosthetic valve 100 can be disposed over deflated valve expansion balloon 664 without the cover shaft disposed therebetween prior to insertion of both into the leaflet opening 52.

With the guest prosthetic valve 100 received within the leaflet opening 52, radially expanding the guest prosthetic valve, as shown in FIG. 25D, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell opening 112 of the guest prosthetic valve 100.

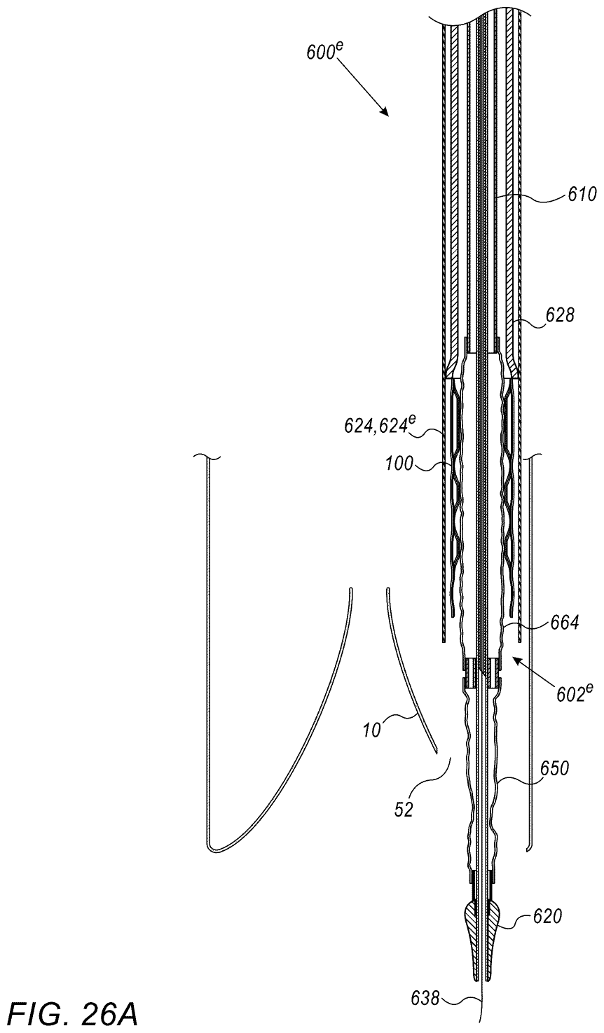

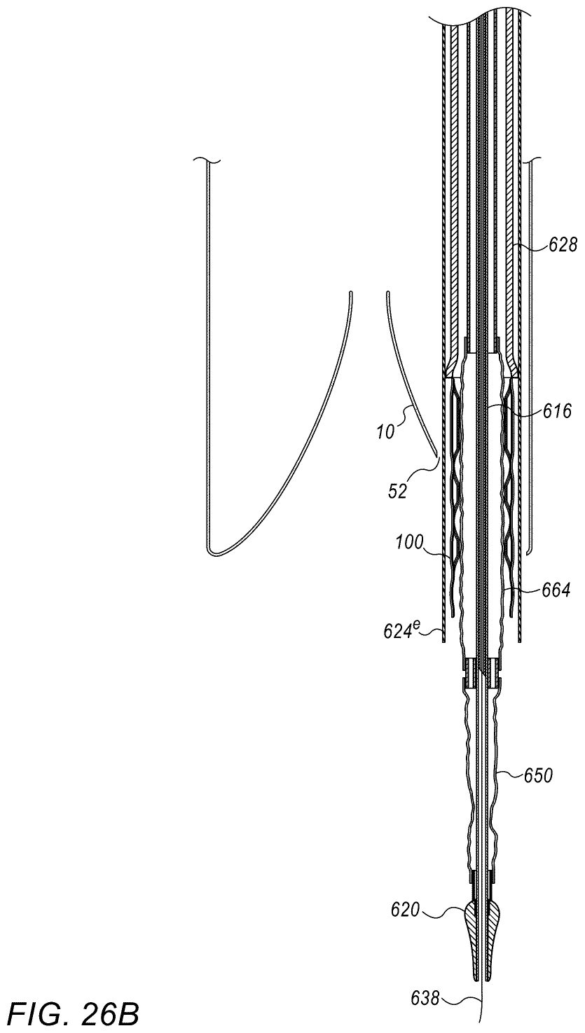

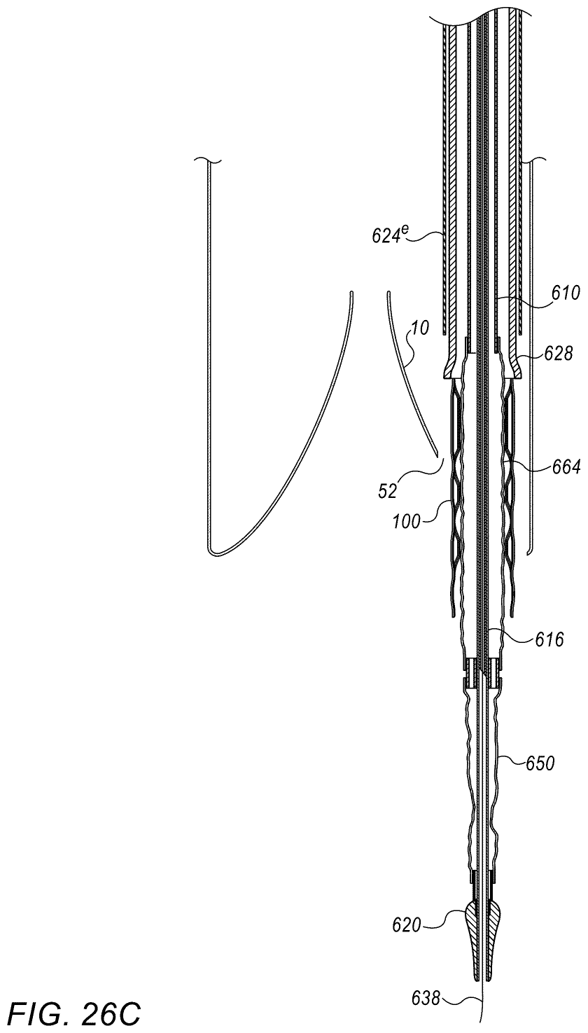

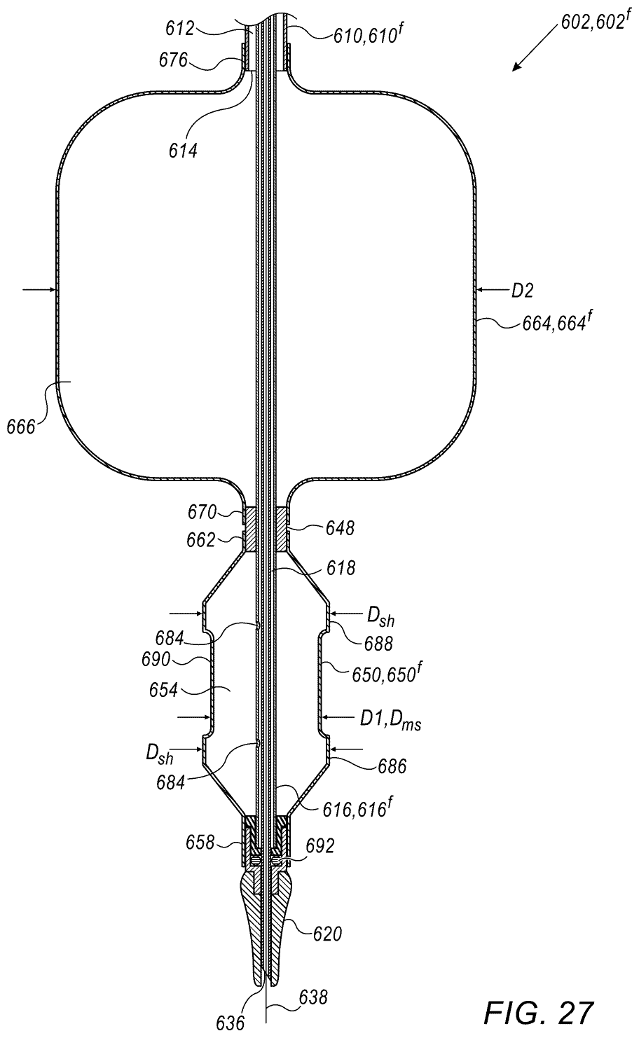

FIGS. 26A-26C show steps which can be performed instead of the steps illustrated in FIGS. 25A-25C, utilizing delivery assembly 600^{e} equipped with delivery apparatus 602^{e}. Delivery apparatus 602^{e} is an exemplary implementation of delivery apparatus 602, and thus includes all of the features described for delivery apparatus 602 throughout the current disclosure, except that the cover shaft 624^{e} is disposed, in the first position, around prosthetic valve 100 instead of between prosthetic valve 100 and valve expansion balloon 664. As shown in FIG. 26A, when the prosthetic valve 100 is positioned in its crimped configuration around deflated valve expansion balloon 664, the distal portion of the cover shaft 624^{e} can be disposed around the prosthetic valve 100, such that prosthetic valve 100 is disposed between the valve expansion balloon 664 and the cover shaft 624^{e}. If prosthetic valve 100 is crimped and advanced through the patient's vasculature during delivery at a position proximal to the valve expansion balloon 664, the distal portion of the cover shaft 624^{e} can cover the valve expansion balloon 664, while the prosthetic valve 100 is retained in a crimped configuration over balloon catheter 610, during delivery, and the push shaft 628 can be utilized to push the prosthetic valve 100 between the valve expansion balloon 664 and the cover shaft 624^{e}, as shown in FIG. 26A, at any stage described hereinabove during which such prosthetic valve 100 advancement can occur.

As shown in FIG. 26B, the deflated valve expansion balloon 664 with the prosthetic valve 100 disposed therearound, and the cover shaft 624^{e} disposed around the prosthetic valve 100, can be then advanced into the leaflet opening 52, and the cover shaft 624^{e} can be retracted to the second position, as shown in FIG. 26C, allowing the valve expansion balloon 664 to be inflated to expand the prosthetic valve 100 in a similar manner to that shown in FIG. 25D.

As mentioned above with respect to FIGS. 25A-25C, it is to be understood that retraction of cover shaft 624^{e} can be performed at any other stage after deflation of hole dilation balloon 650 as shown in FIG. 26A, including prior to advancement of the prosthetic valve 100 into leaflet opening 52, in which case, the prosthetic valve 100 can be disposed over deflated valve expansion balloon 664 without the cover shaft disposed thereover prior to insertion of both into the leaflet opening 52.

While a delivery apparatus 602 that includes an intermediate connector 640, similar to delivery apparatus 602^{a}, is illustrated in FIGS. 23A-26C, it is to be understood that other delivery apparatuses 602 described herein can be similarly utilized. For example, a delivery apparatus 602^{b} in which fluid communication between the cavities 654 and 666 is facilitated through a space formed between connected attachment segments 662, 670 and an inner shaft of the delivery apparatus, such as nosecone shaft 616.

Similarly, a delivery apparatus 602^{d} provided with a unitary balloon 680 can be utilized as described above with respect to FIGS. 23A-26C, wherein a cover shaft 624 (including cover shaft 624^{e}) can be disposed around the proximal portion of the unitary balloon, functioning as the valve expansion balloon 6644, restricting the inflation fluid to inflate the distal portion of the unitary balloon, serving as the hole dilation balloon 6504, for the purpose of forming the leaflet opening 52, after which the cover shaft 624 can be retracted and the proximal portion of the unitary balloon 680, along with the prosthetic valve 100 disposed therearound, can be advanced into the leaflet opening 52, such that re-introducing the inflation fluid into the cavity of the unitary balloon serves to inflate the entire balloon 680, including the proximal portion, so as to expand the prosthetic valve 100 inside leaflet opening 52.

In some examples, the delivery apparatus 602 utilized in a method exemplified in FIGS. 23A-25D can include a hole dilation balloon 650^{c} having a thinner dilation balloon wall 652^{c} relative to expansion balloon wall 672^{c} of the valve expansion balloon 664^{c}, similar to any example of delivery apparatus 602^{c} described above. In such cases, delivery apparatus 602 can still include a cover shaft 624 utilized as described above with respect to FIGS. 23A-26C, or without a cover shaft, relying on the greater thickness T2 of expansion balloon wall 672^{c} to serve as an inflation restriction mechanism, instead of cover shaft 624.

For example, a method of utilizing a delivery apparatus 602^{c} devoid of a cover shaft 624 can include forming a pilot puncture 50 and advancing a deflated hole dilation balloon 650^{c} into the pilot puncture 50 of the host leaflet 10 in a similar manner to that described above with respect to FIGS. 23A-23C, and filling inflation fluid into the dilation balloon cavity 654, for example via balloon catheter opening(s) 614 and expansion balloon cavity 666. The inflation fluid is provided at a pressure which is lower than a predefined threshold pressure, configured to be sufficient to inflate the hole dilation balloon 650^{c} and form leaflet opening 52 in a manner similar to that shown in FIG. 23D, yet low enough to prevent the valve expansion balloon 664^{c} from being inflated to a similar diameter. The higher wall thickness T2 of expansion balloon wall 672^{c} can be set to either resist any inflation of valve expansion balloon 664^{c} at inflation pressures which are lower than the threshold pressure, or to partially resist inflation of valve expansion balloon 664^{c} to allow it to partially expand simultaneously with hole dilation balloon 650^{c}, for implementations in which the prosthetic valve 100 is crimped at a proximal position relative to the valve expansion balloon 664^{c}, and thus will not expand due to partial balloon inflation.

The method can then proceed with deflation of the hole dilation balloon 650^{c} and advancement of the valve expansion balloon 664^{c} with the prosthetic valve 100 disposed therearound, into the leaflet opening 52, in a manner similar to that described above and illustrated in FIGS. 25A-25B, except for not including a cover shaft 624, and thus without the need to include a step of retracting a cover shaft as shown in FIG. 25C. The prosthetic valve 100 can be either crimped over valve expansion balloon 664^{c}, or crimped over a portion of balloon catheter 610 proximal to valve expansion balloon 664^{c}, and advanced by push shaft 628 over valve expansion balloon 664^{c} at any stage prior to positioning the valve expansion balloon 664^{c} in the leaflet opening 52, as described above.

Once positioned in the leaflet opening 52, expansion balloon cavity 666 can be filled with inflation fluid, provided at a pressure which is greater than the predefined threshold pressure, sufficient to inflate the valve expansion balloon 664^{c} and expand the prosthetic valve 100 in a manner similar to that described above and shown in FIG. 25D, mutatis mutandis. These steps can be used in the same manner described above for a unitary balloon 680 that includes a thinner distal portion, configured to function as a hole dilation balloon with smaller thickness T1, and a thicker proximal portion, configured to function as a valve expansion balloon having a greater thickness T2 relative to T1.

In some examples, the method described above for utilizing a delivery apparatus 602 devoid of a cover shaft 624 can be further adapted for utilization of a hole dilation balloon 650 with a thickness T1 of dilation balloon wall 652 which is not necessarily thinner than, and can be, in some examples, substantially equal to, the thickness T2 of expansion balloon wall 672, by controlling the volume of inflation fluid provided into cavities 654, 666. The method can include forming a pilot puncture 50 and advancing a deflated hole dilation balloon 650^{c} into the pilot puncture 50 of the host leaflet 10 in a similar manner to that described above with respect to FIGS. 23A-23C, and filling inflation fluid into the dilation balloon cavity 654, for example via balloon catheter opening(s) 614 and expansion balloon cavity 666. The volume of inflation fluid provided in this stage can be less than a predefined threshold volume, configured to be sufficient to inflate the hole dilation balloon 650 and form leaflet opening 52 in a manner similar to that shown in FIG. 23D, wherein valve expansion balloon 664 can be simultaneously inflated therewith. Simultaneous inflation of both balloons 650, 664 in such cases is performed by providing a limited volume of inflation fluid that will not allow expansion of the valve expansion balloon beyond the maximum diameter D1 of the hole dilation balloon. In such cases, the prosthetic valve 100 can remain in its crimped configuration over a portion of the balloon catheter 610 proximal to valve expansion balloon 664, to prevent it from being expanded as valve expansion balloon 664 is partially inflated.

The method can then proceed with deflation of the hole dilation balloon 650 and the valve expansion balloon 664, pushing the prosthetic valve 100 over valve expansion balloon 664 with push shaft 628, and advancement of the deflated valve expansion balloon 664 with the prosthetic valve 100 disposed therearound, into the leaflet opening 52, in a manner similar to that described above and illustrated in FIGS. 25A-25B, except for not including a cover shaft 624, and thus without the need to include a step of retracting a cover shaft as shown in FIG. 25C.

Once positioned in the leaflet opening 52, a greater volume of inflation fluid can be provided to inflate both balloons 650, 664, wherein inflation of valve expansion balloon 664^{c} expands the prosthetic valve 100 therewith in a manner similar to that described above and shown in FIG. 25D, mutatis mutandis. These steps can be used in the same manner described above for a unitary balloon 680 that includes a uniform thickness of balloon wall along its distal and proximal portions, wherein the limited inflation fluid volume can uniformly expand both portion of the unitary balloon 680 to a diameter which is sufficient to form leaflet opening 52, and higher inflation fluid volume is provided once the proximal portion of the unitary balloon 680 is positioned, with prosthetic valve 100 disposed therearound, in the leaflet opening 52, so as to fully expand all portions of the unitary balloon 680, optionally to the non-uniform profile illustrated in FIG. 22.

For delivery assemblies 600 that include tandem balloons 650, 664 whose cavities 654, 666 are in fluid communication, the hole dilation balloon 650 will usually inflate simultaneously with the valve expansion balloon 664, when the valve expansion balloon 664 is positioned, with prosthetic valve 100 disposed therearound, inside leaflet opening 52. This simultaneous opening of hole dilation balloon 650 does not interfere with the procedure of prosthetic valve 100 expansion and implantation, as the hole dilation balloon 650 is positioned in such cases distal to the host leaflet 10, and is inflated in this positioned to a limited diameter, which does not span the entire opening in which prosthetic valve 100 is expanded within the site of implantation, due to the smaller maximum diameter of the hole dilation balloon 650.

While the methods disclosed herein refer to forming a leaflet opening 52 in a host leaflet 10, prior to positioning and expanding a prosthetic valve 100, it is to be understood that any of the methods can comprise, in some examples, repeating one or more steps disclosed throughout the current specification to form a plurality of punctures and openings in the host valvular structure. For example, steps described above with respect to FIGS. 23A-24B and equivalents thereof, can be performed for forming a first leaflet opening in a first host leaflet, after which the hole dilation balloon can be deflated, for example in a similar manner to that illustrated in FIG. 25A, and the delivery apparatus can be retracted from the first host leaflet and steered toward another host leaflet, after which the same steps can be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be particularly beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In some examples, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. In some examples, the host valvular structure can be modified such that the guest prosthetic valve is implanted by being expanded in a leaflet opening of a first host leaflet that faces the left coronary artery, and such that the second leaflet opening is formed in a second host leaflet that faces the right coronary artery (or vice-versa).

In some examples, forming the first leaflet opening can be performed prior to forming the second leaflet opening. In some examples, forming the second leaflet opening can be performed prior to forming the first leaflet opening. In some examples, the order of forming leaflet openings is chosen such that the final leaflet opening is formed in the host leaflet in which the prosthetic valve 100 is to be positioned and expanded, such as over a valve expansion balloon as described above with respect to FIGS. 25A-26C and equivalents thereof.

It is to be understood that the guest prosthetic valve 100 is not limited to being implanted within an opening 52 of a leaflet. For example, in cases where the hole dilation balloon 650 forms a full tear in a host leaflet that extends to the coaptation edge of the leaflet, the guest prosthetic valve 100 can be positioned at a location between the leaflets of the host valvular structure, for example by retracting the delivery apparatus from the host leaflet in which a leaflet opening is formed, repositioning and readvancing it such that the deflated valve expansion balloon, along with the prosthetic valve 100 disposed thereon, is positioned between the host leaflets, and then inflating the valve expansion balloon to expand the prosthetic valve 100. In some examples, such as in cases where the opening 52 does not form a full tear in the leaflet, the guest prosthetic heart valve can be positioned at a location between the leaflets of the host valvular structure 12 (such that the delivery assembly 600 used to implant to guest prosthetic valve 100 does not extend through the leaflet opening 52) and then expanded. In such cases, the opening 52 may provide sufficient open space through which blood may flow into the coronary ostia, and/or through which additional access devices, such as coronary catheters, can pass during future interventional procedures.

FIG. 27 shows a cross sectional view of an exemplary delivery apparatus 602^{f}. Delivery apparatus 602^{f} is an exemplary implementation of delivery apparatus 602, and thus includes all of the features described for delivery apparatus 602 throughout the current disclosure, except that the dilation balloon cavity 654 and expansion balloon cavity 666 are sealed from each other, such that each of the hole dilation balloon 650^{f} and the valve expansion balloon 664^{f} is separately and independently inflatable. In some examples, delivery apparatus 602^{f} can include an intermediate seal 648 disposed between balloons 650^{f} and 664^{f}. The intermediate seal 648 can be generally structured similarly to intermediate connector 640 described above, but does not include an intermediate channel 642, so as to seal between cavities 654 and 666.

The intermediate seal 648 can be disposed around a shaft of the delivery apparatus 602^{f}. In the illustrated example, the intermediate seal 648 defines an inner surface (similar to connector inner surface 646) that can be coupled to (for example, by gluing, welding, and the like) an outer surface the nosecone shaft 616^{f}. Any of the dilation balloon proximal attachment segment 662 and the expansion balloon distal attachment segment 670 can be similarly coupled to an outer surface of the intermediate seal 648 (which can be similar to outer surface 644). The intermediate seal can have a diameter which is smaller than the maximum diameter of the hole dilation balloon 650^{f} in its inflated state, such that the intermediate seal 648 forms a narrow neck portion between both balloons 650^{f}, 664^{f}. While hole dilation balloon 650 and valve expansion balloon 664^{f} are illustrated in FIG. 27 as separately formed balloons, it is to be understood that both can be similarly implemented as a distal portion of a unitary balloon 680, and a proximal portion of the unitary balloon 680, being greater in diameter than the distal portion, having an intermediate seal disposed between both portions to seal between the cavities 654 and 666 of both portions.

In some examples, delivery apparatus 602^{f} can be provided without an intermediate seal 648, wherein the dilation balloon proximal attachment segment 662 and the expansion balloon distal attachment segment 670 can be similarly coupled to an outer surface of a shaft of the delivery apparatus 602^{f} (for example, by gluing, welding, and the like), such as being attached to an outer surface of the nosecone shaft 616^{f}.

Balloon catheter 610^{f} can comprise at least one balloon catheter opening 614 in fluid communication with expansion balloon cavity 666. The distal end of balloon catheter 610^{f} can terminate at a proximal portion of valve expansion balloon 664^{f}, as illustrated in FIG. 27, or it may extend further distally into expansion balloon cavity 666 (example not illustrated), yet terminate at or proximal to the distal end of valve expansion balloon 664, such as by terminating at or proximal to intermediate seal 648. When balloon catheter 610^{f} terminates proximal to intermediate seal 648, one or more balloon catheter opening(s) 614 can be formed as an open end at the distal end of balloon catheter 610^{f}, defined between a distal edge of balloon catheter 610^{f} and the nosecone shaft 616^{f}, similar to the configuration illustrated in FIG. 27. When balloon catheter extends to some length further distally into expansion balloon cavity 666, one or more balloon catheter opening(s) 614 can be formed on the sidewall of balloon catheter 610^{f}, exposed laterally to expansion balloon cavity 666.

Nosecone shaft 616^{f} extends through dilation balloon cavity 654, and since balloon catheter 610^{f} terminates proximal to dilation balloon cavity 654, the outer surface of the nosecone shaft 616^{f} is exposed to dilation balloon cavity 654. At least one nosecone shaft side opening 684 is formed on the sidewall of nosecone shaft 616^{f}, exposed laterally to dilation balloon cavity 654. Thus, one or more nosecone shaft side opening(s) 684 provide fluid communication between the nosecone shaft lumen 618 and dilation balloon cavity 654, through which inflation fluid can flow to fill dilation balloon cavity 654. When a perforating member 630, such as a hollow needle, extends through nosecone shaft 616^{f}, the inner diameter D_{nc} of the nosecone shaft 616^{f} is greater than the outer diameter Dₚₘ of perforating member 630, allowing flow of inflation fluid through the space between the inner surface of nosecone shaft 616^{f} and the outer surface of perforating member 630. When a perforating member 630 is not passed through nosecone shaft 616^{f}, the inner diameter D_{nc} of nosecone shaft 616^{f} is greater than the diameter D_{gw} of guidewire 638, allowing flow of inflation fluid through the space between the inner surface of nosecone shaft 616^{f} and the guidewire 638.

In some examples, delivery apparatus 602^{f} can further include a distal seal 692 disposed around the nosecone shaft 616^{f} distal to dilation balloon cavity 654, such as between dilation balloon cavity 654 and nosecone 620, to distally seal dilation balloon cavity 654.

Each of the balloon catheter lumen 612 and the nosecone shaft lumen 618 can be separately fed by inflation fluid, for example by being fluidly coupled to separate fluid inflation sources (for example, different syringes), or to different outlets of a common fluid inflation source, controllable to independently feed each of the lumens 612, 618. Thus, inflation fluid provided through nosecone shaft lumen 618 can flow, through nosecone shaft side opening(s) 684, into dilation balloon cavity 654, so as to inflate the hole dilation balloon 650, without affecting the valve expansion balloon 664. Inflation fluid can be similarly drawn from dilation balloon cavity 654 to deflate the hole dilation balloon 650^{f}. Likewise, inflation fluid provided through balloon catheter lumen 612 can flow, through balloon catheter opening(s) 614, into expansion balloon cavity 666, so as to inflate the valve expansion balloon 664^{f}, without affecting the hole dilation balloon 650^{f}. Inflation fluid can be similarly drawn from expansion balloon cavity 666 to deflate the valve expansion balloon 664^{f}.

Since each balloon 650, 664 can be independently inflated, without affecting the other balloon, delivery apparatus 602^{f} can be provided without a cover shaft 624. A method of utilizing delivery apparatus 602^{f} can include forming a pilot puncture 50 and advancing a deflated hole dilation balloon 650 into the pilot puncture 50 of the host leaflet 10 in a similar manner to that described above with respect to FIGS. 23A-23C, and filling inflation fluid into the dilation balloon cavity 654, for example feeding inflation fluid into nosecone shaft lumen 618, passing through nosecone shaft side opening(s) 684 into dilation balloon cavity 654. This allows hole dilation balloon 650^{f} to inflate and form a leaflet opening 52 in a similar manner to that shown in FIG. 23D. Inflation fluid is not fed into balloon catheter lumen 612 yet, allowing the valve expansion balloon 664^{f} to remain deflated at this stage.

The method can then proceed with deflation of the hole dilation balloon 650, for example by drawing the inflation fluid from dilation balloon cavity 654 back through nosecone shaft lumen 618, to result in a deflated hole dilation balloon 650^{f} in a similar manner to that shown in FIG. 25A.

If the prosthetic valve 100 is crimped around balloon catheter 610^{f} at a portion proximal to valve expansion balloon 664, the push shaft 628 can be used to push prosthetic valve 100 toward and around valve expansion balloon 664 after deflating the hole dilation balloon 650, or at any other stage preceding deflation of hole dilation balloon 650. The valve expansion balloon 664 with the prosthetic valve 100 disposed therearound can be then advanced into the leaflet opening 52, in a manner similar to that described above and illustrated in FIG. 25B, except for not necessarily including a cover shaft 624, and thus without the need to include a step of retracting a cover shaft as shown in FIG. 25C.

Once positioned in the leaflet opening 52, inflation fluid can be provided through balloon catheter lumen 612 and balloon catheter opening(s) 614, filling expansion balloon cavity 666 so as to inflate the valve expansion balloon 664 and expand the prosthetic valve 100 in a manner similar to that described above and shown in FIG. 25D, mutatis mutandis.

In some examples, as shown in FIG. 27, the hole dilation balloon 650 can include a proximal shoulder 688 and a distal shoulder 686 disposed on both ends of dilation balloon main segment 690. Proximal shoulder 688 can extend between dilation balloon proximal tapering segment 660 and dilation balloon main segment 690, and distal shoulder 686 can extend between dilation balloon distal tapering segment 656 and dilation balloon main segment 690. As shown, each of the shoulders 686, 688 can have an outer shoulder diameter Dₛₕ which is greater than a uniform outer diameter Dₘₛ of the dilation balloon main segment 690 (i.e., Dₛₕ>Dₘₛ) in the inflated state of hole dilation balloon 650. This configuration advantageously prevents the edges of the formed leaflet opening 52 from slipping axially away, either in the proximal or the distal direction, from dilation balloon main segment 690.

While two shoulders 686, 688 are illustrated in FIG. 27, in some examples, only a distal shoulder 686 can be provided, without a proximal shoulder 688, and in some examples, only a proximal shoulder 688 can be provided, without a distal shoulder 686. While both shoulders 686, 688 are shown to have substantially equal diameters Dₛₕ in the illustrated example, it is to be understood that each of the shoulders 686, 688 can be formed, in some examples, with a different outer diameter, yet the diameters of both shoulders 686, 688 will be greater than the diameter of dilation balloon main segment 690. When a hole dilation balloon 650 includes any of the shoulders 686, 688, any reference herein to a diameter D1 thereof being less than the diameter D2 of a valve expansion balloon 664, refers to the diameter Dₘₛ of the main segment 690 of hole dilation balloon 650.

While a hole dilation balloon 650 that includes shoulders 686, 688 disposed on both ends of its main segment 690 is illustrated in FIG. 27 with respect to delivery apparatus 602^{f}, it is to be understood that this is shown by way of illustration and not limitation, and that a hole dilation balloon 650 that includes shoulders 686, 688 according to any example above can be similarly implemented with any other example of delivery apparatus 602 described herein, including exemplary delivery apparatus 602^{a}, 602^{b}, 602^{c} and/or 602^{d} illustrated throughout FIGS. 19-22.

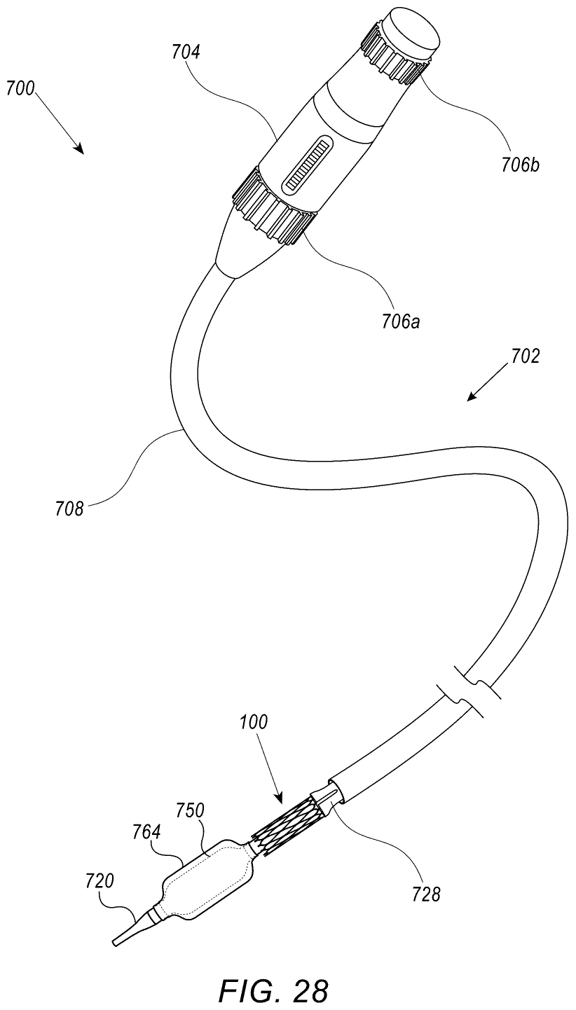

FIG. 28 illustrates an exemplary delivery assembly 700 that includes an exemplary delivery apparatus 702 adapted to deliver a balloon expandable prosthetic valve 100, such as prosthetic valve 100 described above with respect to FIGS. 2A-2B. The delivery apparatus 702 can be similar to delivery apparatus 602, except that instead of being provided with a tandem balloon arrangement as described for the different examples of delivery apparatus 602, delivery apparatus 702 has a balloon-in-balloon arrangement, with an inner balloon 750 disposed within the outer balloon 764. The inner balloon 750 is functionally analogous to hole dilation balloon 650, while the outer balloon 764 is functionally analogous to the valve expansion balloon. In some examples, the spatial arrangement of the two balloons 650, 664 is concentric and symmetrical.

Delivery apparatus 702 can be devoid of a cover shaft. Other components of the delivery apparatus 702 are in all respects similar to components of delivery apparatus 602 described above, and in the interest of brevity will not be described further. For example, delivery apparatus 702 can include: a handle 704 with knobs 706 (for example, knobs 706a and 706^{b}), an outer delivery shaft 708, a balloon catheter 710 defining a balloon catheter lumen 712 and comprising at least one balloon catheter opening 714, a nosecone 720, a nosecone shaft 716 defining a nosecone shaft lumen 718, a push shaft 728, a perforating member 730, and a guidewire 738, which are structurally and functionally similar, respectively, to: the handle 604 with knobs 606 (for example, knobs 606a and 606^{b}), the outer delivery shaft 608, the balloon catheter 610 defining balloon catheter lumen 612 and comprising at least one balloon catheter opening 614, the nosecone 620, the nosecone shaft 616 defining nosecone shaft lumen 618, the push shaft 628, perforating member 630, and the guidewire 638.

The prosthetic valve 100 can be carried by the delivery apparatus 702 during delivery in a crimped state, and expanded by inflation of outer balloon 764 to secure it in a native heart valve annulus (such as aortic annulus 24) or against a previously implanted prosthetic valve. The prosthetic valve 100 can be initially crimped over the balloon catheter 710, proximal to the outer balloon 764. During delivery, the distal end portion of the push shaft 728 is positioned proximal to the outflow end 106 of prosthetic valve 100. The delivery assembly 700 can further include a guidewire 738 extending through the balloon catheter 710, which refers to any example in which the guidewire 738 can pass through a lumen of any shaft extending through the balloon catheter 710, such as a lumen of a nosecone shaft and/or a lumen of a perforating member, as will be described in further detail below.

When reaching the host valve, the delivery apparatus 702 can be utilized to modify at least one host leaflet 10 by positioning the deflated balloons 750, 764 inside a pilot puncture 50 formed in the host leaflet 10, and inflating the inner balloon 750, as will be described in further detail below. The push shaft 728 can be utilized to position the prosthetic valve 100 over the outer balloon 764, such that when the outer balloon 764 is inflated inside the host leaflet 10, it expands the prosthetic valve 100. Once the prosthetic valve 100 is expanded to its functional diameter within a native annulus or within a previously implanted host prosthetic valve, the outer balloon 764, as well as the inner balloon 750, can be deflated, and the delivery apparatus 702 can be retrieved from the patient's body.

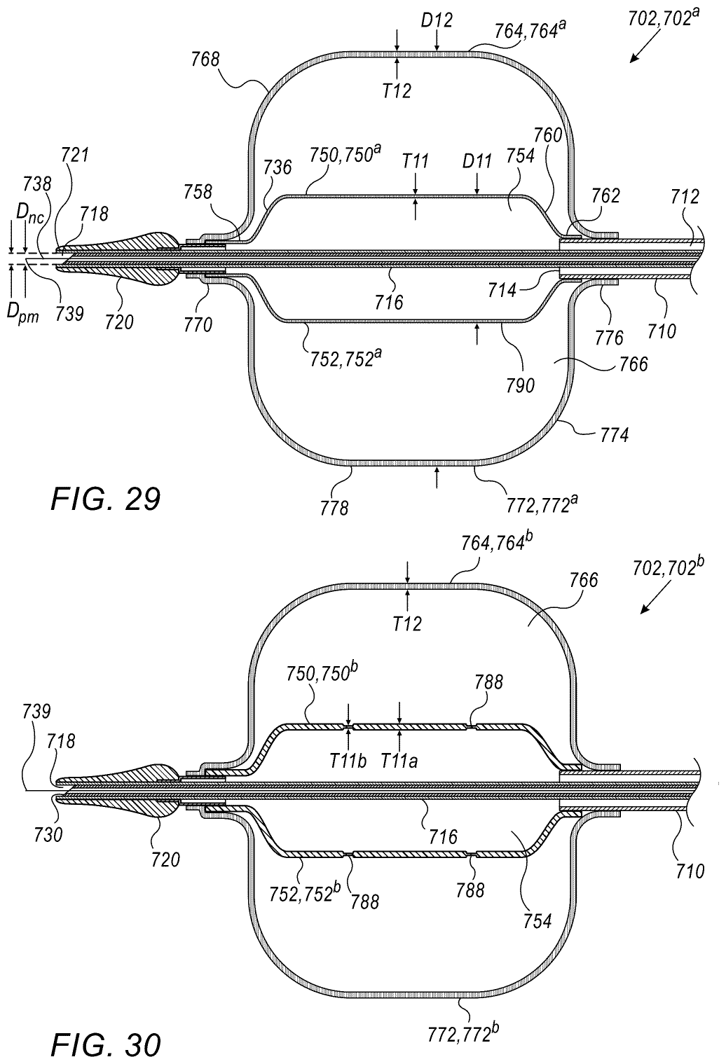

Each of the inner balloon 750 and outer balloon 764 is configured to transition between a deflated state and an inflated state. FIGS. 29-30 show cross-sectional views of a distal portion of some examples of delivery assemblies 700, illustrated in a fully inflated state of both the inner balloon 750 and the outer balloon 764. As shown, the inner balloon 750 is smaller in diameter than the outer balloon 764 in their fully inflated states. In some examples, the maximum diameter D11 of inner balloon 750 in its inflated state is less than 75% of the maximum diameter D12 of the outer balloon 764 in its fully inflated state. In some examples, the maximum diameter D11 of inner balloon 750 in its inflated state is less than 50% of the maximum diameter D12 of the outer balloon 764 in its fully inflated state. In some examples, the maximum diameter D11 of inner balloon 750 in its inflated state is less than 40% of the maximum diameter D12 of the outer balloon 764 in its fully inflated state. In some examples, the maximum diameter D11 of inner balloon 750 in its inflated state is less than 30% of the maximum diameter D12 of the outer balloon 764 in its fully inflated state.

In some examples, the maximum diameter D11 to which the inner balloon 750 can be inflated is equal to or less than 12 mm. In some examples, the maximum diameter D11 is equal to or less than 10 mm. In some examples, the maximum diameter D11 is equal to or less than 8 mm. In some examples, the maximum diameter D12 to which the outer balloon 764 can be inflated is at least 25 mm. In some examples, the maximum diameter D12 is at least 30 mm. In some examples, the maximum diameter D12 is at least 40 mm.

The inner balloon 750 can comprise an inner balloon distal tapering segment 756 configured to assume a tapering configuration in the inflated state of the balloon 750, extending from a narrower diameter at a distal end thereof to a wider diameter at its proximal end, and an inner balloon main segment 790 extending proximally from the inner balloon distal tapering segment 756. In some examples, the inner balloon main segment 790 can have a uniform diameter along its length in the inflated state. In some examples, as shown in FIGS. 29-30, the inner balloon 750 can further comprise an inner balloon proximal tapering segment 760 tapering from a larger diameter of the inner balloon main segment 790 to a smaller diameter at the proximal end of inner balloon proximal tapering segment 760.

The outer balloon 764 can comprise an outer balloon proximal tapering segment 774 configured to assume a tapering configuration in the inflated state of the outer balloon 764, extending from a narrower diameter at a proximal end thereof to a wider diameter at its distal end, and an outer balloon main segment 778 extending distally from the outer balloon proximal tapering segment 774. In some examples, the outer balloon main segment 778 can have a uniform diameter along its length in the inflated state. In some examples, as shown in FIGS. 29-30, the outer balloon 764 can further comprise an outer balloon distal tapering segment 768 tapering from a larger diameter of the outer balloon main segment 778 to a smaller diameter at the distal end of outer balloon distal tapering segment 768.

Inner balloon distal tapering segment 756 can include an inner balloon distal attachment segment 758 that can be secured to a component of the delivery apparatus 702, such as a distal portion of balloon catheter 710, a distal portion of the nosecone shaft 716, a proximal portion of nosecone 720, or to components coupled to nosecone 720. Inner balloon proximal tapering segment 760 can include an inner balloon proximal attachment segment 762 that can be secured to a component of the delivery apparatus 702, such as a balloon catheter 710 or nosecone shaft 716.

Outer balloon distal tapering segment 768 can include an outer balloon distal attachment segment 770 that can be secured to a component of the delivery apparatus 702, such as a distal portion of balloon catheter 710, a distal portion of the nosecone shaft 716, a proximal portion of nosecone 720, or to components coupled to nosecone 720. In some examples, outer balloon distal attachment segment 770 can at least partially overlap and be attached to the inner balloon distal attachment segment 758. Outer balloon proximal tapering segment 774 can include an outer balloon proximal attachment segment 776 that can be secured to a component of the delivery apparatus 702, such as balloon catheter 710. In some examples, outer balloon proximal attachment segment 776 can at least partially overlap and be attached to the inner balloon proximal attachment segment 762. Any of the attachment segments 758, 762, 770, 776 can be secured to another component of the delivery apparatus 702 by force fitting, heat pressing, welding or a suitable adhesive.

As further shown in FIGS. 29-30, the nosecone shaft 616 extends through the balloon catheter lumen 712, and defines a nosecone shaft lumen 718 extending along the entire length of the nosecone shaft 716 and nosecone 720, through which guidewire 738 can pass, such that the entire delivery apparatus 702 can be advanced toward the treatment region over the guidewire 738. In some examples, a perforating member 730 can extend through the balloon catheter 710, such as by extending through balloon catheter lumen 712 and/or a lumen of a shaft extending through balloon catheter 710, such as through a nosecone shaft lumen 718 of a nosecone shaft 716. In some examples, the perforating member 730 is axially movable relative to another component of the delivery apparatus 702, such as the inner balloon 750 and/or outer balloon 764. In some examples, a perforating member 730 can extend through nosecone shaft lumen 718, and be axially movable relative to nosecone shaft 716. The perforating member 730 can define a perforating member lumen 736, through which the guidewire 738 can extend.

Nosecone shaft 716, balloon catheter 710 and optional outer delivery shaft 708 can be formed from any of various suitable materials, such as nylon, braided stainless steel wires, or a polyether block amide (commercially available as Pebax®). In some examples, balloon catheter 710 and optional outer delivery shaft 708 have longitudinal sections formed from different materials in order to vary the flexibility of the shafts along their lengths. In some examples, nosecone shaft 716 has an inner liner or layer formed of Teflon® to minimize sliding friction with a guidewire 738 and/or a perforating member 730.

Nosecone shaft 716 can be similar to nosecone shaft 616, defining an inner diameter D_{nc}, which is the diameter of nosecone shaft lumen 718. Perforating member 730 can be similar to perforating member 630, defining an outer diameter Dₚₘ, and may be implemented as a hollow needle, through which guidewire 738, similar to guidewire 638 and having a guidewire diameter Dew, can extend. As mentioned above with respect to delivery apparatus 602, while many conventional nosecone shafts are designed to have an inner diameter that merely allows a guidewire 738 to pass therethrough, a delivery apparatus 702 that further includes a perforating member 730 passable through the nosecone shaft lumen 718, can include a nosecone shaft 716 designed to have a relatively larger inner diameter D_{nc}, to allow both the perforating member 730 and the guidewire 738 extending therethrough, to freely pass and be axially movable within the nosecone shaft lumen 718. Thus, a perforating member 730 can have perforating lumen 736 dimensioned similarly to conventional nosecone shaft lumens, to allow for similar axial movement of a guidewire 738 therein, necessitating the inner diameter D_{nc} of non-conventional nosecone shaft 716 to be, in turn, greater in size, and specifically, greater than the outer diameter Dₚₘ of the perforating member 730.

Inner balloon 750 comprises an inner balloon wall 752 having a wall thickness T11 and defining an internal cavity 754. Outer balloon 764 comprises an outer balloon wall 772 having a wall thickness T12 and defining an internal cavity 766. In some examples, the inner balloon 750 is configured to transition from a sealed configuration, in which the inner balloon cavity 754 is fluidly sealed from the portion of outer balloon cavity 766 between the inner balloon wall 752 and the outer balloon wall 772, to an unsealed configuration in which the inner balloon cavity 754 is in fluid communication with the outer balloon cavity 766, such that inflation fluid may freely flow through inner balloon cavity 754 to outer balloon cavity 766. In such cases, the inner balloon 750 is retained in the sealed configuration during delivery and during formation of the leaflet opening 52, and can transition to the unsealed configuration prior to prosthetic valve 100 expansion. It is to be understood that the term “outer balloon cavity 766” refers to the space bound between the outer balloon wall 772 and the inner balloon wall 752, when the inner balloon 750 is in the sealed configuration.

The outer diameter of nosecone shaft 716 can be sized such that an annular space is formed within balloon catheter lumen 712 between balloon catheter 710 and nosecone shaft 716 along the length of balloon catheter 710. This annular space can be in fluid communication with one or more balloon catheter openings 714 exposed to the internal cavity 754 of the inner balloon 750, which can be in fluid communication with a fluid source (for example, a syringe or a pump) that can inject an inflation fluid (for example, saline) into inner balloon cavity 754. In this way, fluid from the fluid source can flow through balloon catheter lumen 712, and into inner balloon cavity 754 via balloon catheter opening(s) 714, which serves to inflate the inner balloon 750, for example during formation of leaflet opening 52. When the inner balloon 750 is in the unsealed configuration, inflation fluid can flow further into outer balloon cavity 766, which serves to inflate the outer balloon 764 and expand and deploy a prosthetic valve 100 disposed thereon.

When the inner balloon 750 is in the sealed configuration, the pressure of the inflation fluid within the inner balloon 750 may provide the force that allows the main segment 690 of hole dilation balloon 650 to dilate a leaflet opening 52 in a manner that will be further described below. The pressure of the inflation fluid within outer balloon 764, for example when the inner balloon 750 is in the unsealed configuration, may provide the force that allows the main segment 778 of outer balloon 764 to expand a prosthetic valve 100 disposed thereon. Further, the balloon catheter lumen 712 may be configured to withdraw fluid from inner balloon cavity 754 and/or outer balloon cavity 766 through the balloon catheter opening(s) 714, to deflate the inner balloon 750 and/or outer balloon 764, respectively.

While balloon catheter 710 is shown to terminate at a proximal end of inner balloon 750 in FIGS. 29-30, in some examples, balloon catheter 710 can extend farther in the distal direction, through a portion or through the entire length of inner balloon cavity 754, and one or more balloon catheter opening(s) 714 can be formed on the sidewall of balloon catheter 710, exposed laterally to inner balloon cavity 754.

In some examples, the inner balloon 750 is configured to tear or burst to transition from the sealed configuration to the unsealed configuration. FIG. 29 shows a cross sectional view of an exemplary delivery apparatus 702^{a}. Delivery apparatus 702^{a} is an exemplary implementation of delivery apparatus 702, and thus includes all of the features described for delivery apparatus 702 throughout the current disclosure, except that the inner balloon wall 752^{a} is weakened relative to the outer balloon wall 772^{a}. In some examples, the wall thickness T11 of inner balloon wall 752^{a} is thinner than the wall thickness T12 of the outer balloon wall 772^{a}. Inner balloon wall 752^{a} can have a uniform thickness T11, or a non-uniform thickness, in which case a reference to a thinner balloon wall 752^{a} can refer to the minimal thickness of inner balloon wall 752^{a}. The thickness T11 of inner balloon wall 752^{a} is configured to withstand an inflation pressure which is less than a pressure threshold, and to tear or burst when the pressure within inner balloon cavity 754 exceeds the pressure threshold.

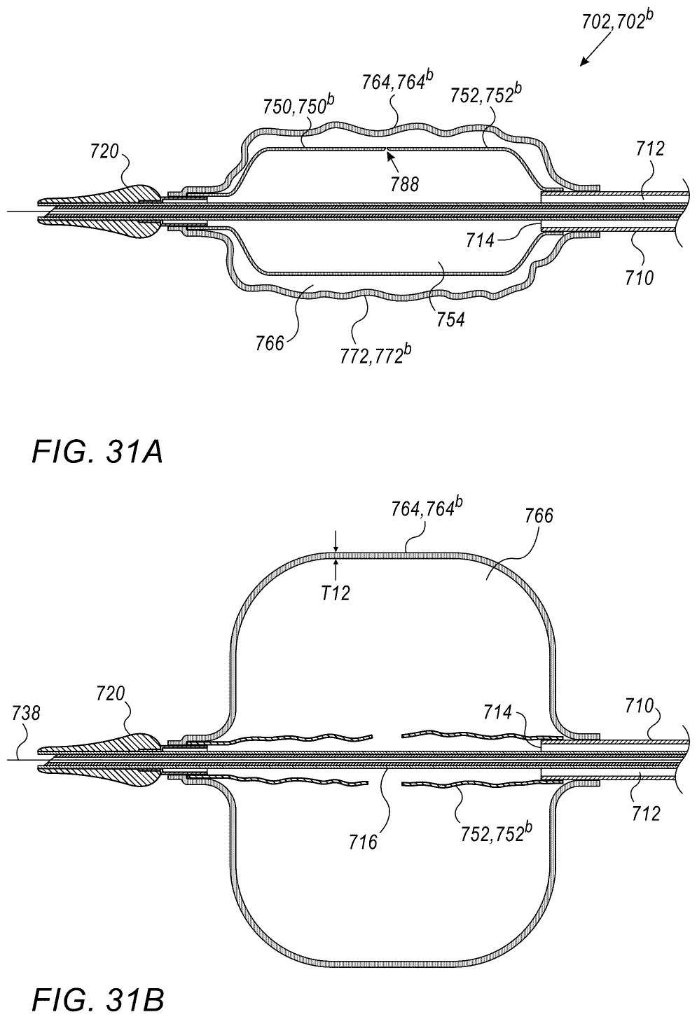

FIGS. 31A-31B show inflation of balloons 750, 764, which can be similar to balloons 750^{a}, 764^{a}, in a sealed configuration (FIG. 31A) and an unsealed configuration (FIG. 31B) of the inner balloon 750. As shown in FIG. 31A, the inner balloon 750^{a} can be fully inflated when filled by inflation fluid at a pressure that is less than a predefined pressure threshold, kept at the sealed configuration. However, when inflation fluid exceeds the pressure threshold, the inner balloon wall 752^{a} is torn, transitioning the inner balloon 750^{a} to the unsealed configuration in which the inflation fluid can fill, through the torn portion, the outer balloon cavity 766, as shown in FIG. 31B. The higher thickness T12 of the outer balloon wall 772^{a} allows it to withstand the higher pressure (exceeding the pressure threshold), facilitating, in turn, inflation of the outer balloon 764^{a}.

In some examples, instead or in addition to having different thicknesses, the balloons 750, 764 can be formed of different materials, such that the material from which the outer balloon wall 772 is made is more tear resistant than the material from which the inner balloon wall 752 is made. For example, the material properties of the inner balloon wall 752 can be configured to tear when the inflation pressure exceed the predefined pressure threshold, while the material properties of the outer balloon wall 772 can be configured to withstand this higher pressure, to facilitate outer balloon 764 inflation. In such examples, the wall thicknesses T11 and T12 can be substantially equal to each other, though non-equal wall thicknesses can be also combined with differing material properties.

FIG. 30 shows a cross sectional view of an exemplary delivery apparatus 702^{b}. Delivery apparatus 702^{b} can be similar to delivery apparatus 702^{a}, with the exception that inner balloon wall 752^{b} has a non-uniform thickness, and more specifically, includes at least one weakened region 788. In some examples, a weakened region 788 is implemented as a thinned region having a wall thickness T11^{b} which is less than the wall thickness T11a of at least one other region of the inner balloon wall 752^{b}. FIG. 30 illustrates an example that includes a plurality of weakened regions 788, but it is to be understood that any number, shape and size of thinned or otherwise weakened regions 788 is contemplated, including a single weakened region 788. When a weakened region 788 is implemented as a thinned region, its thickness T11^{b} is less than the thickness T11^{b} of other region(s) of inner balloon wall 752^{b}, and can be also thinner than the thickness T12 of the outer balloon wall 772^{b}. The majority of the inner balloon wall 752 can be formed to have a thickness T11a, which can be, in some examples, substantially equal to the thickness T12 of the outer balloon wall 772^{b}. The thickness T11^{b} at the thinned region(s) 788 is configured to withstand an inflation pressure which is less than a pressure threshold, and to tear or burst when the pressure within inner balloon cavity 754 exceeds the pressure threshold.

While illustrated as a thinned region in FIG. 30, it is to be understood that a weakened region 788 can be implemented in any other manner by which region 788 is weaker than at least one other region of the inner balloon wall 752^{b}, meaning that a weakened region 788 is configured to withstand an inflation pressure which is less than a pressure threshold, and to tear or burst when the pressure within inner balloon cavity 754 exceeds the pressure threshold, while any other non-weakened region can remain intact. For example, a weakened region 788 can be formed from a different material than other, non-weakened regions of the inner balloon wall 752^{b}. Combinations of weakening modes are also contemplated, such as forming a weakened region 788 from a different material, as well as having smaller thickness such as T11a.

FIGS. 31A-31B, described above with respect to delivery apparatus 702^{a}, can be similarly representative of the sealed and unsealed configuration of delivery apparatus 702^{b}. For example, as shown in FIG. 31A, the inner balloon 750^{b} can be fully inflated when filled by inflation fluid at a pressure that is less than a predefined pressure threshold, kept at the sealed configuration. However, when inflation fluid exceeds the pressure threshold, the weakened region(s) 788 of inner balloon wall 752^{b} is torn, transitioning the inner balloon 750^{b} to the unsealed configuration in which the inflation fluid can fill, through the torn portion, the outer balloon cavity 766, as shown in FIG. 31B. The higher thickness T12 and/or material properties of the outer balloon wall 772^{b} allow it to withstand the higher pressure (exceeding the pressure threshold), facilitating, in turn, inflation of the outer balloon 764^{b}.

While inner balloons 750^{a} and 750^{b} of delivery assemblies 700700^{a} and 700^{b} are configured to passively transition from a sealed to an unsealed configuration, by tearing or bursting in response to an elevated inflation pressure, in some examples, a delivery apparatus 702 of delivery assembly 700 can include a mechanism controllable by an operator of the delivery apparatus 702, to transition the inner balloon 750 to the unsealed configuration.

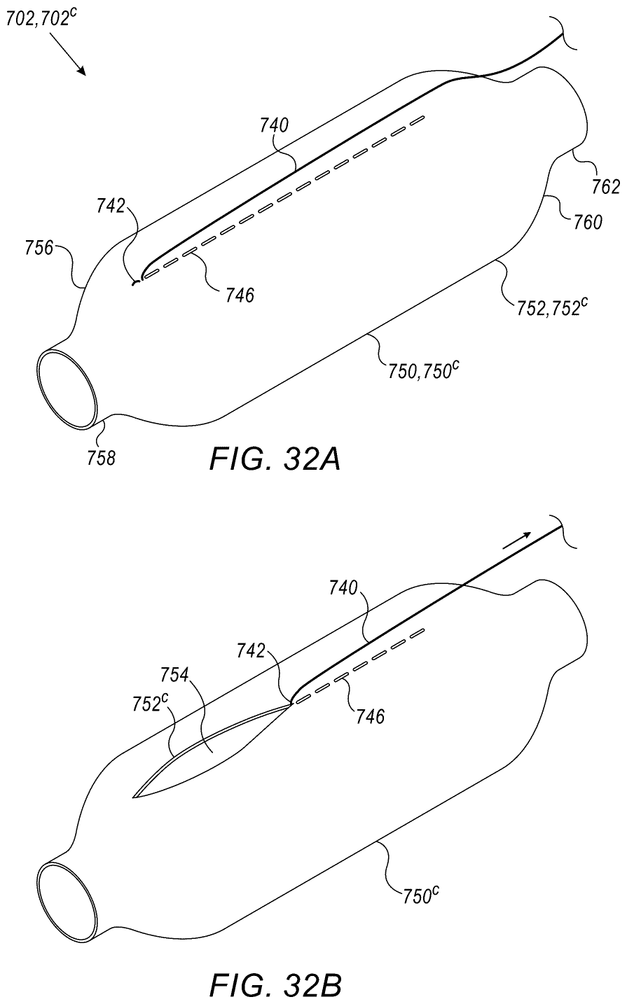

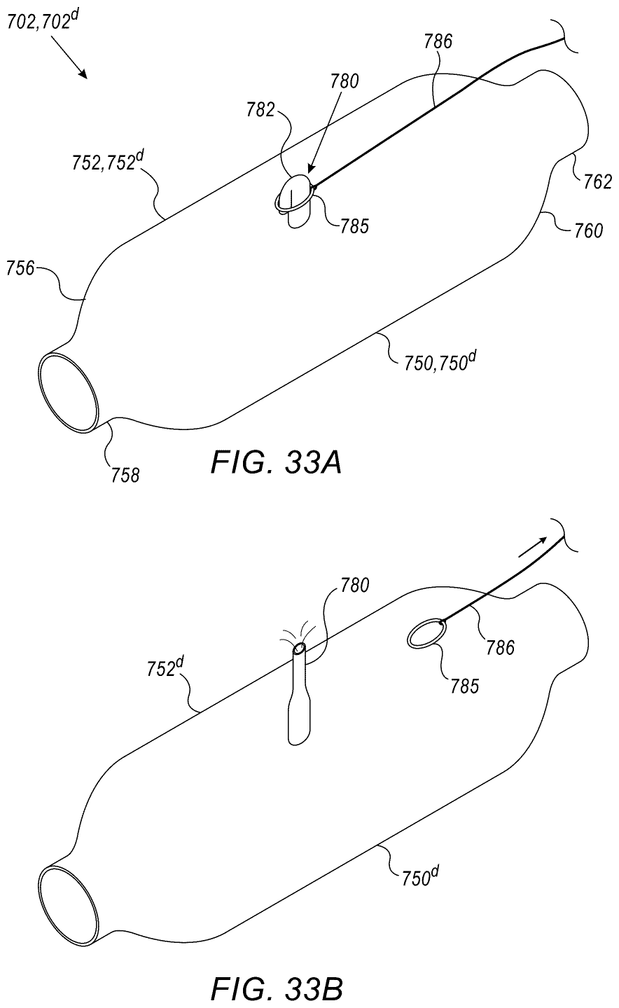

FIGS. 32A and 32B show an inner balloon 750^{c} of a delivery apparatus 702^{c} in a sealed configuration and an unsealed configuration, respectively. For clarity, inner balloon 750^{c} is illustrated without the outer balloon or other components of the delivery apparatus in FIGS. 32A-32B. Delivery apparatus 702^{a} is an exemplary implementation of delivery apparatus 702, and thus includes all of the features described for delivery apparatus 702 throughout the current disclosure, except that the inner balloon wall 752^{c} further comprises a tear-line 746 to which a ripcord 740 is attached at a distal end 742 thereof. The tear-line 746 can be formed as a relatively weakened line extending along a certain length of the inner balloon wall, such as a thinned line or a score-line (as long as the score-line does not include apertures that pass through the whole thickness of the inner balloon wall), which can still maintain structural integrity during inflation of the inner balloon 750 without tearing or bursting, as long as the ripcord 740 is not actively pulled. The ripcord distal end 742 is attached to a distal end of the tear-line 746, and runs proximally from the inner balloon wall 752, along delivery apparatus 702^{c}, for example into the handle 704.

The handle 704 (not shown in FIG. 32A-32B) can include a controllable mechanism for pulling the ripcord 740, such that when the operator pulls the ripcord 740 (for example, controllable by an appropriate knob 706), it causes the tear-line 746 to gradually tear from its distal end to its proximal end, thereby transitioning the inner balloon 750^{c} to the unsealed configuration, as shown in FIG. 32B. The ripcord 740 can be provided as a wire, string, suture, and the like. In some examples, the ripcord 740 can extend from the inner balloon 750^{c} to the handle 704 through a lumen of a shaft of delivery apparatus 702^{c}, such as a lumen of balloon catheter 710^{c} or a lumen of outer delivery shaft 708. In some examples, at least one of the shafts or catheters of delivery apparatus 702^{c}, such as balloon catheter 710^{c}, can be a multi-lumen shaft, including a separate lumen through which ripcord 740 can extend.

FIGS. 33A and 33B show an inner balloon 750^{d} of a delivery apparatus 702^{d} in a sealed configuration and an unsealed configuration, respectively. For clarity, inner balloon 750^{d} is illustrated without the outer balloon or other components of the delivery apparatus in FIGS. 33A-33B. Delivery apparatus 702^{a} is an exemplary example of delivery apparatus 702, and thus includes all of the features described for delivery apparatus 702 throughout the current disclosure, except that the inner balloon 750^{c} further comprises a flexible port 780 extending sideways from inner balloon wall 7524. The flexible port 780 can be a tubular member, optionally made from the same material and integrally formed with the inner balloon wall 752^{a}, that can extend radially or angularly away from the inner balloon wall 752^{d}.

In the sealed configuration shown in FIG. 33A, the flexible port is folded over itself, forming a port fold 782 that block fluid communication therethrough, such that inflation fluid cannot escape from the inner balloon cavity 754 through the folded port 780. A loop 785 can be disposed around the port fold 782 to maintain it in this state, as long as the loop 785 is not actively displaced. A release cord 786 can be coupled to the loop 785, and run proximally therefrom along delivery apparatus 7024, for example into the handle 704.

The handle 704 (not shown in FIGS. 33A-33B) can include a controllable mechanism for pulling the release cord 786, such that when the operator pulls the release cord 786 (for example, controllable by an appropriate knob 706), it causes the loop 785 to slip from the flexible port 780, thereby transitioning the inner balloon 750^{d} to the unsealed configuration, as shown in FIG. 33B, allowing inflation fluid to freely flow from the inner balloon cavity 754 through the unfolded port 780. The release cord 786 can be provided as a wire, string, suture, and the like. The loop 785 can be a suture loop, a band, a ring (for example, in the form of an O-ring), and the like. In some examples, the release cord 786 can extend from the loop 785 to the handle 704 through a lumen of a shaft of delivery apparatus 702^{a}, such as a lumen of balloon catheter 710^{d} or a lumen of outer delivery shaft 708. In some examples, at least one of the shafts or catheters of delivery apparatus 702^{c}, such as balloon catheter 7104, can be a multi-lumen shaft, including a separate lumen through which release cord 786 can extend.

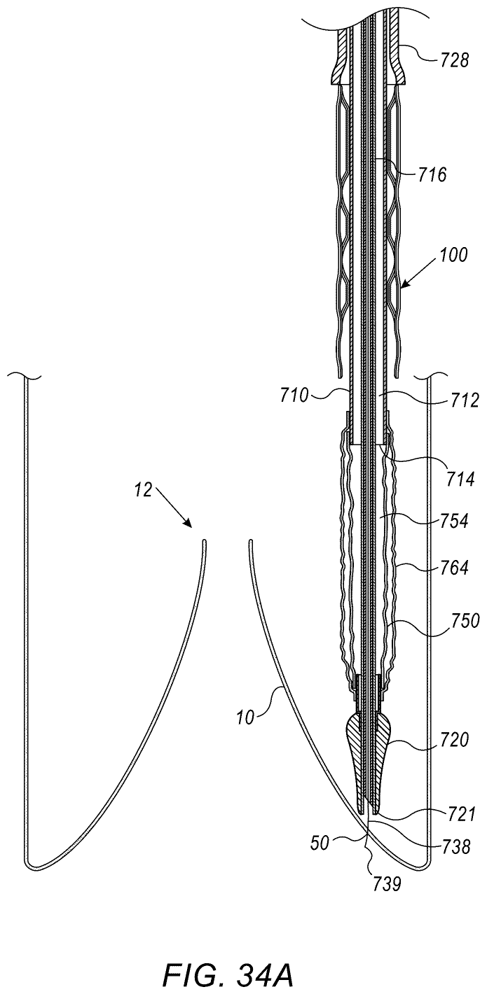

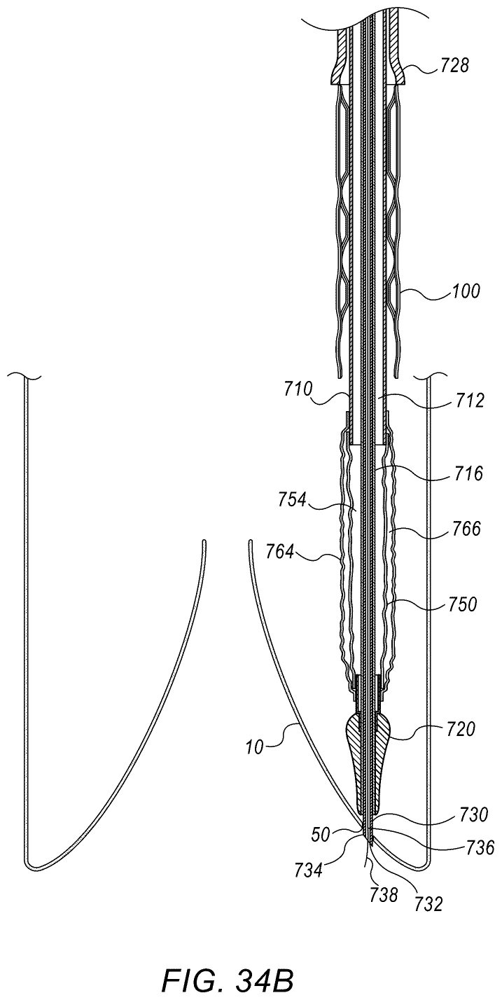

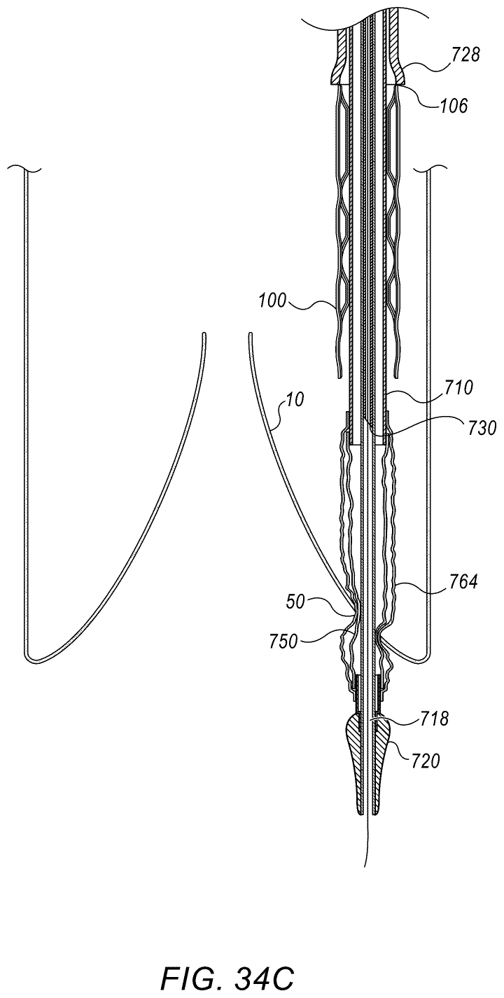

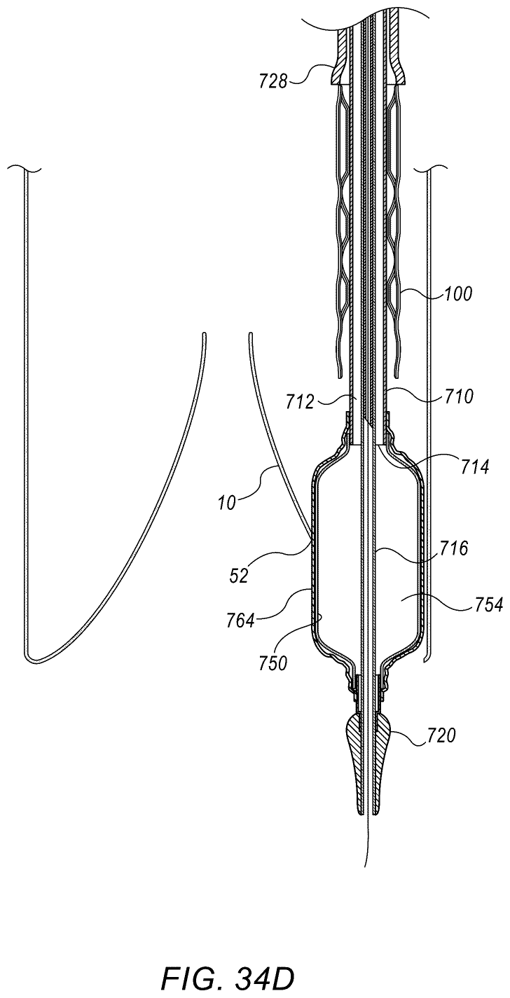

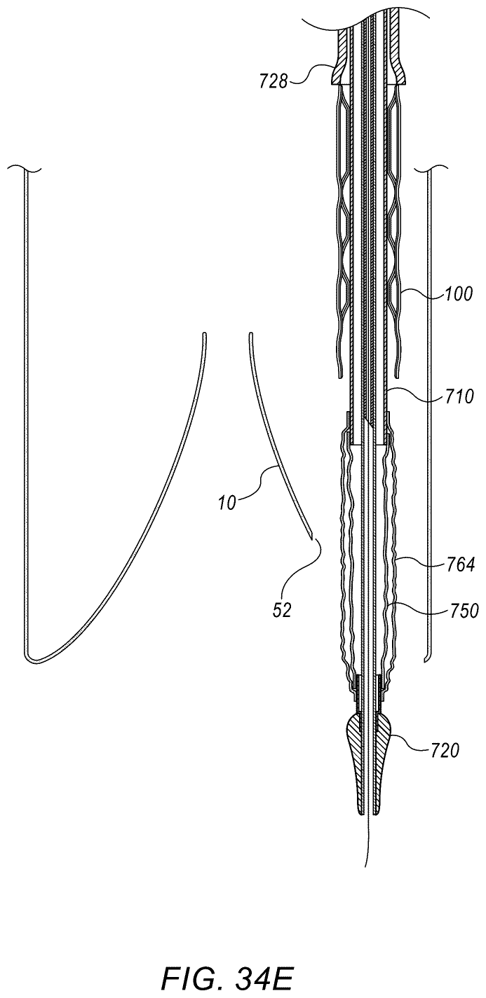

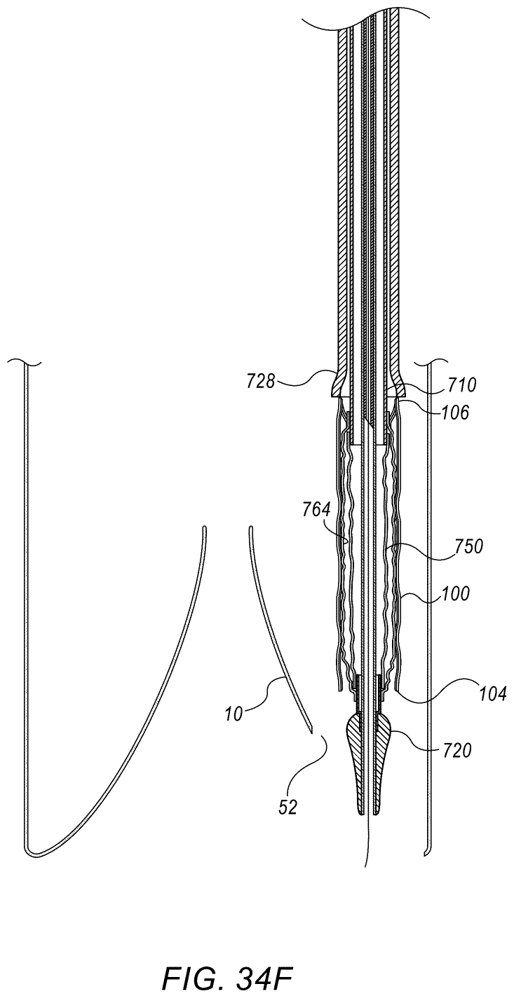

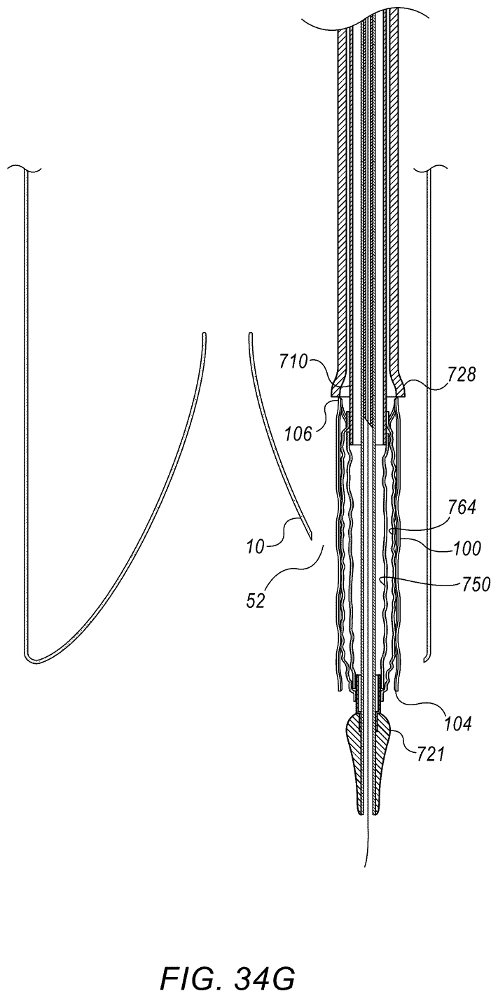

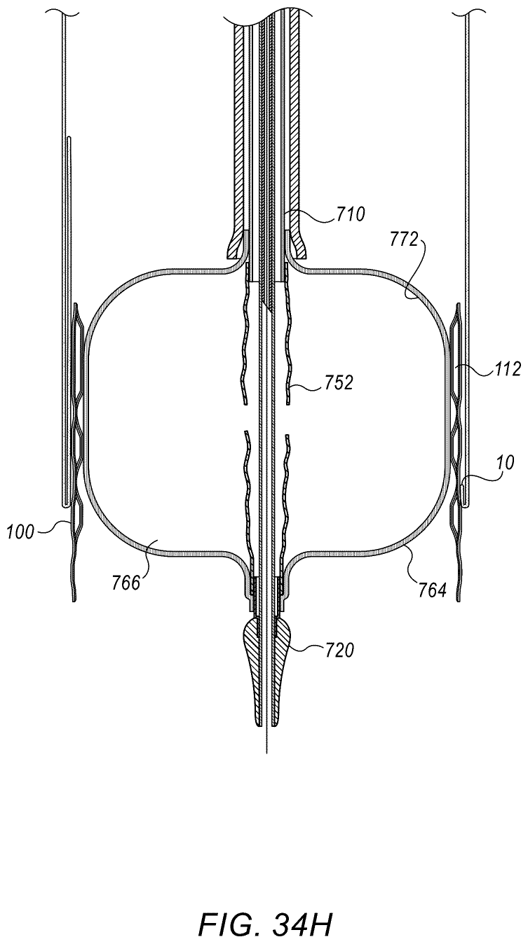

FIGS. 34A-34H illustrate some steps in a method for utilizing a delivery assembly 700 for forming a leaflet hole inside a host leaflet, an implanting a guest prosthetic valve inside the host valvular structure. The delivery assembly 700 can be used to perforate, cut, and/or tear a host leaflet 10, such as a native leaflet 30 or a prosthetic valve leaflet 114 of a previously implanted prosthetic valve. While delivery assembly 700^{a} comprising delivery apparatus 702^{a} is illustrated throughout FIGS. 34A-34H, it is to be understood that other examples of delivery apparatus 702 described in the current specification can be used in a similar manner, as will be further elaborated below.

The distal end portion of the delivery apparatus 702, including nosecone 720, is configured to be inserted into a patient's vasculature, such as within an ascending aorta, and to be advanced towards the host leaflet 10, wherein the guidewire can optionally pierce through the host leaflet 10 as shown in FIG. 34A. Positioning the delivery apparatus 702 relative to the host leaflet 10 may comprise advancing the delivery apparatus 702 toward the leaflet via guidewire 738. As mentioned above, the delivery apparatus 702 can further include a perforating member 730, which can be also referred to as a lacerating member 730. Perforating member 730 comprises a distal end portion 732 configured to pierce a host leaflet 10 of a host valvular structure 12 to form a pilot puncture 50 in the host leaflet 10, when a distal end portion 732 is positioned distal to the inner balloon 750 and/or outer balloon 764.

In some examples, the distal end portion 732 of perforating member 730 is configured to be selectively translated in the proximal or distal directions relative to another component of the delivery apparatus 702, such as any of the inner balloon 750, the outer balloon 764 and/or the nosecone 720. In some examples, the nosecone shaft 716 and the perforating member 730 are configured to be movable axially relative to each other in the proximal and distal directions. The perforating member 730 can be coupled to a handle 704. The handle 704 can have one or more actuators (for example, in the form of rotatable knobs 706) that are operatively coupled to the perforating member 730 to facilitate axial movement thereof. In such examples, the distal end portion 732 can be configured to pierce a host leaflet 10 when axially translated to a position which is distal to any of the inner balloon 750, the outer balloon 764 and/or the nosecone 720. In some examples, the distal end portion 732 is not necessarily configured to be axially translatable relative to the inner balloon 750, in which case it is positioned distal to the inner balloon 750 at all times.

As shown in FIG. 34B, the perforating member 730 is configured to puncture the host leaflet 10 to form a pilot puncture 50 within host leaflet 10. Subsequent to forming the pilot puncture 50, and as shown in FIG. 34C, the deflated balloons 750, 764 may be inserted within the pilot puncture 50. The distal end portion 732 of the perforating member 730 can be concealed within a lumen of the delivery apparatus 702, being positioned proximal to a distal end 721 of the nosecone 720 during delivery, as shown in FIG. 34A, to avoid damage that may be caused to internal anatomical structures of the patient's body due to accidental contact with an optionally sharp distal end portion 732. The distal end portion 732 can be then pushed toward and through the host leaflet 10 to form the pilot puncture 50 as shown in FIG. 34B, after which it can be retracted back to re-conceal the distal end portion 732.

In some examples, the perforating member 730 can be retracted so as to position the distal end portion 732 proximal to the nosecone distal end 721 after forming the pilot puncture 50 and prior to advancing the nosecone 720 to place inner balloon 750 inside the pilot puncture 50, relying on the nosecone distal end 721 being small enough to allow insertion of the tapering nosecone 720 through pilot puncture 50. In some examples, the distal end portion 732 of the perforating member 730 remains distal to the nosecone distal end 721 during advancement of the nosecone 720 and inner balloon 750 through the pilot puncture 50, after which it is retracted to conceal the perforating member 730, positioning its distal end portion 732 proximal to the nosecone distal end 721, as illustrated in FIG. 34C.

With the inner balloon 750 received within the pilot puncture 50, inflating the inner balloon 750 to transition it from the radially deflated state (FIG. 34C) to the radially inflated state (FIG. 34D) can expand the pilot puncture 50 to form a leaflet opening 52 that is sized to receive the prosthetic valve 100 in the radially compressed or crimped configuration.

In some examples, inflating the inner balloon 750 within the host leaflet 10 serves to increase a diameter of the pilot puncture 50 such that the resulting leaflet opening 52 is a hole with an increased diameter relative to the pilot puncture 50. Since the inner balloon 750 is in a sealed configuration at this stage, as it is inflated, it pushes against the outer balloon wall 772, causing the outer balloon wall 772 to press, in turn, against the borders of the leaflet opening 52 during formation thereof, as shown in 22D. In some examples in which the leaflet opening 52 is a hole, the leaflet opening 52 may be a substantially circular hole. In other examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the inner balloon 750 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet). As further shown, the prosthetic valve 100 is positioned in a crimped configuration over the balloon catheter 710 at a position proximal to the balloons 750, 764, up to and during formation of the leaflet opening 52.

The delivery apparatus 702 may be configured to form the leaflet opening 52 in any of a variety of host valvular structures 12. In the example of FIGS. 34A-34H, the host valvular structure 12 can be the valvular structure 113 of a previously implanted prosthetic valve, such as the prosthetic valve 100a of FIG. 3. In such examples, using the delivery assembly 700 as described herein to form the leaflet opening 52 in a previously implanted prosthetic valve may be followed by steps for implanting a guest prosthetic valve 100^{b} within the previously implanted prosthetic valve 100a (for example, via a ViV procedure).

Similarly, the host valvular structure 12 in the example of FIGS. 34A-34H can be a valvular structure 29 of a native heart valve, such as the native aortic valve 20 shown in FIGS. 2A-2B. In such examples, the perforating member 730 can be configured to puncture a native leaflet 30 of the native aortic valve 20. In other examples, the host valvular structure and/or the native valve may refer to another valve of a patient's heart, such as a mitral valve, a pulmonary valve, or a tricuspid valve.

In some examples, the perforating member 730 may include and/or be a needle, such as a spring-loaded needle and/or a Veress needle. As shown in FIGS. 34A-34C, the distal end portion 732 of the perforating member 730 can terminate at an angled surface 734. The angled surface 734 can have a sharp cutting edge to facilitate piercing the host leaflet 10 when the needle is pressed against the leaflet.

Delivery apparatus 702 may include any of a variety of features to facilitate positioning the nosecone 720 and/or the perforating member 730 relative to the host leaflet 10. For example, the nosecone shaft 716, balloon catheter 710, outer delivery shaft 708, and/or the perforating member 730, may be pre-formed, shaped, and/or curved so as to be directed and/or angled toward the host leaflet 10 when positioned in the vicinity of the host valvular structure. Furthermore, one or more shafts of delivery apparatus 702, such as outer delivery shaft 708, can have a steering mechanism (for example, a pull wire and a corresponding adjustment mechanism in the handle 704) to steer or adjust its distal end.

As further mentioned above, in some examples, the perforating member 730 can comprise a perforating member lumen 736, configured to accommodate a guidewire 738 that can extend through the perforating member lumen 736. In such examples, the guidewire 738 can be inserted into the patient's vasculature, and then the perforating member 730 and/or other shafts of the delivery apparatus 702 may be advanced toward the host leaflet 10 over the guidewire 738.

In some examples, the guidewire 738 can be used as a perforating or lacerating member for forming a pilot puncture 50. In such examples, the guidewire 738 can be a relatively stiff wire having a distal tip 739 configured to pierce the host leaflet 10 when the guidewire 738 is pressed against the leaflet. In some examples, the guidewire 738 can include a radio-frequency (RF) energy delivery tip 739 to assist with penetration through the leaflet tissue. For this purpose, a suitable RF energy device may be coupled to the guidewire 738, and the RF energy device can apply the RF energy to the tip 739 of the guidewire 738 to penetrate the host leaflet 10. In any examples disclosed herein wherein a guidewire is used to puncture a leaflet, the guidewire can be coupled to a source of RF energy that applies RF energy to the tip of the guidewire. When the guidewire 738 is used to pierce the leaflet 10, the perforating member 730 in the form of a needle can be omitted, or it can be used in combination with the guidewire 738 that forms an initial puncture in the leaflet 10. For example, the guidewire 738 can be used to form an initial pilot puncture 50 (see FIG. 34A), after which the perforating member 730 can be advanced through the leaflet to form a slightly larger pilot puncture (see FIG. 34B) for subsequent advancement of the balloons 750, 764 through the host leaflet 10.

In some examples, the guidewire 738 is used as a perforating member without any additional separate perforating member, such as a needle, disposed thereover, such that the guidewire 738 can be utilized as the sole component that forms the pilot puncture 50, allowing other components of the delivery apparatus, such as nosecone 720, to pass through the pilot puncture 50, in a manner similar to that illustrated in FIG. 34B, but without the needle 730.

In some examples, the guidewire 738 is used as a perforating member that can be used in addition to perforating member (for example, needle) 730, such that the guidewire 738 can form an initial puncture via a sharp tip 739 or an RF energy delivery tip 739, as illustrated in FIG. 34A, followed by penetration of the perforating member 730 into the leaflet 10 to form the pilot puncture 50, or a pilot puncture 50 which is greater in size than an initial puncture formed by the guidewire tip 739, as shown in FIG. 34B.

In some examples, the guidewire tip 739 is not necessarily sharp enough or otherwise configured to puncture through the host leaflet 10, in which case the guidewire 738 can be utilized for advancement of the delivery apparatus 702 toward the valvular structure 12, but terminate in proximity of the host leaflet 10 without piercing through it (for example, remaining above host leaflet 10 instead of passing through the tissue as shown in FIG. 34A), and the distal end portion 732 of perforating member 730 can be then advanced toward and into the host leaflet 10, to form the pilot puncture 50 in a similar manner to that illustrated in FIG. 34B.

After the inner balloon 750 is inflated to radially push outwards the outer balloon wall 772 therewith, so as to form the leaflet opening 52 as shown in FIG. 34D, the inner balloon 750 is deflated, as shown in FIG. 34E, allowing the push shaft 728 to be utilized to distally advance the crimped prosthetic valve 100 toward and around the outer balloon 764, for example as shown in FIG. 34G.

In some examples, the balloon 750, 764 are retracted away from the leaflet opening, positioned proximal to the host leaflet 10, after inner balloon 750 deflation and prior to placement of the prosthetic valve 100 over the outer balloon 764, after which the push shaft 728 is utilized to push the prosthetic valve 100 over and around the outer balloon 764, as shown in FIG. 34F, after which both balloons 750, 764 can be advanced, along with prosthetic valve 100 disposed thereover, back into leaflet opening 52, as shown in FIG. 34G. Retraction of balloons 750, 764 after formation of the leaflet opening 52, as shown in FIG. 34F, can be advantageous in that it allows the prosthetic valve 100 to be maneuvered more conveniently as it is placed over the outer balloon 764. Nevertheless, in some examples, retraction shown in FIG. 34F is skipped, such that the prosthetic valve 100 is pushed over the outer balloon 764, as shown in FIG. 34G, right after inner balloon deflation (shown in FIG. 34E).

At this stage, a passive or active mechanism of the types described above with respect to FIGS. 29-33B can be utilized to transition the inner balloon 750 from the sealed configuration to an unsealed configuration, such that delivering inflation fluid into the inner balloon cavity 754, and therefrom into the outer balloon cavity 766, allows the outer balloon 764 to inflate and expand the prosthetic valve 100, as shown in FIG. 34H. As described in more detail below, expanding the guest prosthetic valve 100 to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame 102 of the guest prosthetic valve 100.

In some examples, a delivery apparatus 702 that includes an inner balloon 750 configured to tear or burst in response to internal fluid pressure exceeding a predetermined pressure threshold, such as inner balloon 750^{a} or inner balloon 750^{b}, can be utilized in the method illustrated in FIGS. 34A-34H. In such cases, inflation fluid can be provided into inner balloon cavity 754 during the stage of leaflet opening 52 formation shown in FIG. 34D, at a pressure that does not exceed the predetermined pressure threshold, while in FIG. 34H, the inflation fluid is supplied at a pressure higher than the threshold pressure, which serves to tear the inner balloon 750 and allow the inflation fluid to fill the outer balloon cavity 766, so as to inflate the outer balloon 764 and expand prosthetic valve 100.

In some examples, delivery apparatus 702^{c}, described above with respect to FIGS. 32A-32B, can be utilized in the method illustrated in FIGS. 34A-34H. In such cases, inflation fluid can be provided into inner balloon cavity 754 during the stage of leaflet opening 52 formation shown in FIG. 34D, while the tear-line 746 remains intact and the inner balloon 750^{c} is kept in a sealed configuration. After the prosthetic valve 100 is positioned, over the outer balloon 764c, within the leaflet opening 52, the ripcord 740 can be pulled so as to form a slit or tear along tear-line 746, allowing inflation fluid to fill the outer balloon cavity 766 as shown in FIG. 34H, so as to inflate the outer balloon 764^{c} and expand prosthetic valve 100.

In some examples, delivery apparatus 7024, described above with respect to FIGS. 33A-33B, can be utilized in the method illustrated in FIGS. 34A-34H. In such cases, inflation fluid can be provided into inner balloon cavity 754 during the stage of leaflet opening 52 formation shown in FIG. 34D, while the flexible port 780 remains folded over itself by loop 785 disposed therearound, keeping the inner balloon 750^{d} in a sealed configuration. After the prosthetic valve 100 is positioned, over the outer balloon 7644, within the leaflet opening 52, the release cord 786 can be pulled so remove the loop 785 from the flexible port 780, allowing the flexible port 780 to unfold such that the inflation fluid can fill the outer balloon cavity 766 as shown in FIG. 34H, so as to inflate the outer balloon 764^{a} and expand prosthetic valve 100.

As mentioned above, the method can comprise, in some examples, repeating one or more steps to form a plurality of punctures and openings in the host valvular structure. For example, steps described above with respect to FIGS. 34A-34D, can be performed for forming a first leaflet opening in a first host leaflet, after which the inner balloon can be deflated, for example in a similar manner to that illustrated in FIG. 34E, and the delivery apparatus can be retracted from the first host leaflet and steered toward another host leaflet, after which the same steps can be repeated to form a second leaflet opening within the second host leaflet. The procedure can be optionally repeated to form further leaflet openings, such as a third leaflet opening in a third host leaflet.

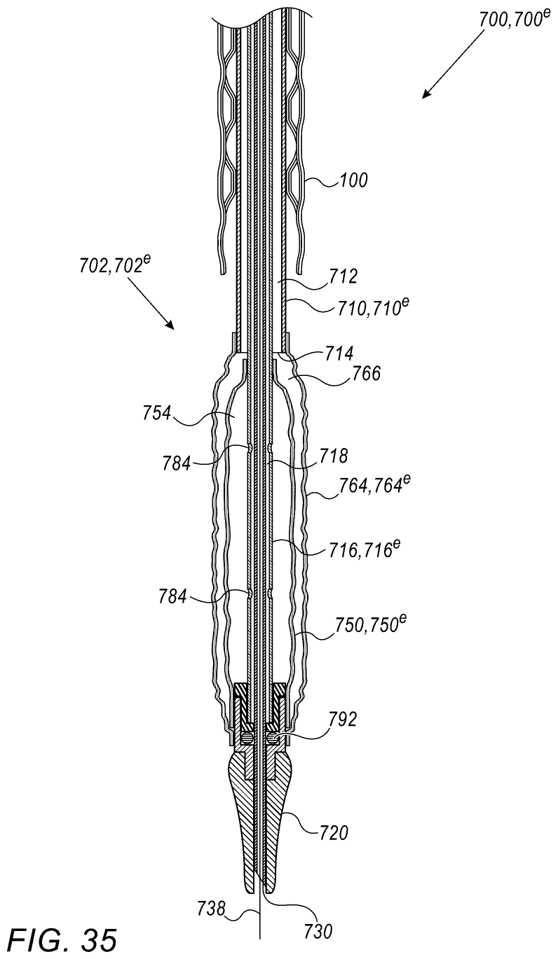

FIG. 35 shows a cross sectional view of an exemplary delivery apparatus 702^{e}. Delivery apparatus 702^{e} is an exemplary implementation of delivery apparatus 702, and thus includes all of the features described for delivery apparatus 702 throughout the current disclosure, except that the inner balloon cavity 754 and outer balloon cavity 766 are sealed from each other throughout the whole procedure. That is to say, inner balloon 750^{e} is kept in the sealed configuration at all times, without transitioning to an unsealed configuration, wherein each of the inner balloon 750^{e} and the outer balloon 664^{e} is separately and independently inflatable.

Balloon catheter 710^{e} can comprise at least one balloon catheter opening 714 in fluid communication with outer balloon cavity 766. The distal end of balloon catheter 710^{e} can terminate at a proximal portion of outer balloon 764^{e}, as illustrated in FIG. 35, wherein the one or more balloon catheter opening(s) 714 can be formed as an open end at the distal end of balloon catheter 710^{e}, defined between a distal edge of balloon catheter 710^{e} and the nosecone shaft 716^{e}.

Nosecone shaft 716^{e} extends through inner balloon cavity 754, and since balloon catheter 710^{e} terminates proximal to inner balloon cavity 754, the outer surface of the nosecone shaft 716^{e} is exposed to inner balloon cavity 754. The inner balloon proximal attachment segment 762 can be coupled to the nosecone shaft 716^{e}, distal to balloon catheter opening(s) 714, such that the balloon catheter opening(s) 714 are not exposed to the inner balloon cavity 754.

At least one nosecone shaft side opening 784 is formed on the sidewall of nosecone shaft 716^{e}, exposed laterally to inner balloon cavity 754. Thus, one or more nosecone shaft side opening(s) 784 provide fluid communication between the nosecone shaft lumen 718 and inner balloon cavity 754, through which inflation fluid can flow to fill inner balloon cavity 754. When a perforating member 730, such as a hollow needle, extends through nosecone shaft 716^{e}, the inner diameter D_{nc} of nosecone shaft 716^{e} is greater than the outer diameter Dₚₘ of perforating member 730, allowing flow of inflation fluid through the space between the inner surface of nosecone shaft 716^{e} and the outer surface of perforating member 730. When a perforating member 730 is not passed through nosecone shaft 716^{e}, the inner diameter D_{nc} of nosecone shaft 716^{e} is greater than the diameter D_{gw} of guidewire 738, allowing flow of inflation fluid through the space between the inner surface of nosecone shaft 716^{e} and the guidewire 738.

In some examples, delivery apparatus 702^{e} can further include a distal seal 792 disposed around the nosecone shaft 716^{e} distal to inner balloon cavity 754, such as between inner balloon cavity 754 and nosecone 720, to distally seal inner balloon cavity 754.

Each of the balloon catheter lumen 712 and the nosecone shaft lumen 718 can be separately fed by inflation fluid, for example by being fluidly coupled to separate fluid inflation sources (for example, different syringes), or to different outlets of a common fluid inflation source, controllable to independently feed each of the lumens 712, 718. Thus, inflation fluid provided through nosecone shaft lumen 718 can flow, through nosecone shaft side opening(s) 784, into inner balloon cavity 754, so as to inflate the inner balloon 750^{e}, without affecting the outer balloon 764. Inflation fluid can be similarly drawn from inner balloon cavity 754 to deflate the inner balloon 750^{e}. Likewise, inflation fluid provided through balloon catheter lumen 712 can flow, through balloon catheter opening(s) 714, into outer balloon cavity 766, so as to inflate the outer balloon 764^{e}, without affecting the inner balloon 750^{e}. Inflation fluid can be similarly drawn from outer balloon cavity 766 to deflate the outer balloon 764^{e}.

The method described above with respect to FIGS. 34A-34H can be adapted for utilization of delivery assembly 700^{e}. Specifically, formation of pilot puncture 50 and advancement of deflated balloons 750^{e}, 764^{e} into pilot puncture 50 of host leaflet 10 can be performed in the same manner described above with respect to FIGS. 34A-34C. This is followed by filling inflation fluid into the inner balloon cavity 754, for example feeding inflation fluid into nosecone shaft lumen 718, passing through nosecone shaft side opening(s) 784 into inner balloon cavity 754. This allows inner balloon 750^{e} to inflate and form a leaflet opening 52 in a similar manner to that shown in FIG. 34D. Inflation fluid is not fed into balloon catheter lumen 712 yet, allowing the outer balloon 764^{e} to remain deflated at this stage.

The method can then proceed with deflation of the inner balloon 750^{e}, for example by drawing the inflation fluid from inner balloon cavity 754 back through nosecone shaft lumen 718, to result in a deflated inner balloon 750^{e} in a similar manner to that shown in FIG. 34E.

The push shaft 728 can be then utilized to push the prosthetic valve 100 over the outer balloon 764^{e}, either while the outer balloon 764^{e} is still positioned within leaflet opening 52, as shown in FIG. 34G, or by first retracting balloons 750^{e}, 764^{e} out of the leaflet opening 52 and pushing the prosthetic valve 100 over the outer balloon 764^{e} as shown in FIG. 34F, followed by repositioning of balloons 750^{e}, 764^{e}, along with prosthetic valve 100 disposed around the outer balloon 764^{e}, back into the leaflet opening 52, as illustrated in FIG. 34G.

Once positioned in the leaflet opening 52, inflation fluid can be provided through balloon catheter lumen 712 and balloon catheter opening(s) 714, filling outer balloon cavity 766 so as to inflate the outer balloon 764^{e} and expand the prosthetic valve 100 in a manner similar to that described above and shown in FIG. 34H, mutatis mutandis.

With the guest prosthetic valve 100 received within the leaflet opening 52, radially expanding the guest prosthetic valve, as shown in FIG. 34H, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell opening 112 of the guest prosthetic valve 100.

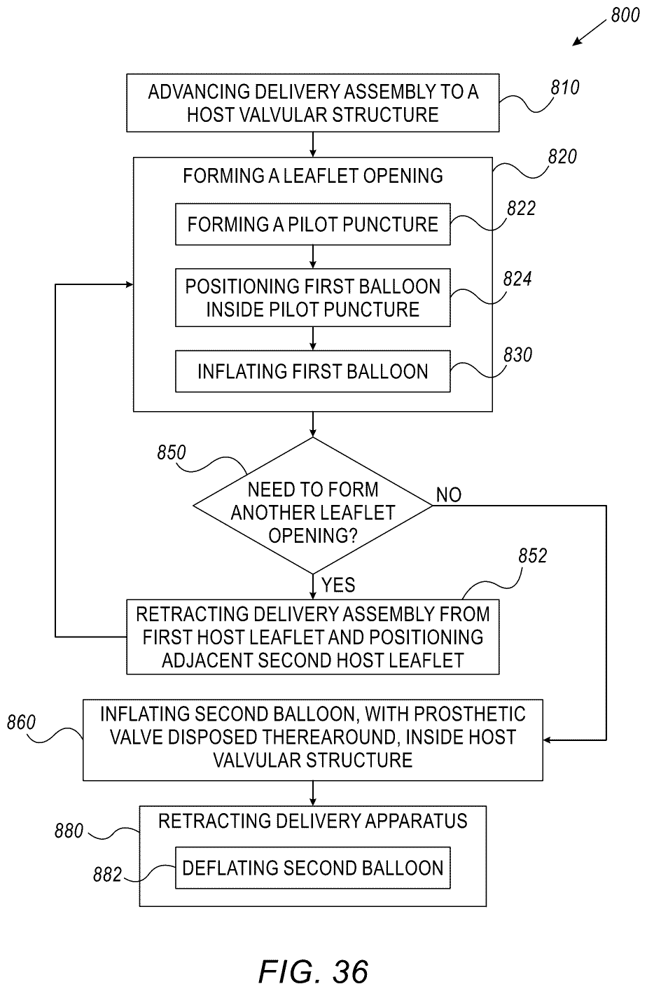

FIG. 36 is a flow chart depicting examples of a method 800 of implanting a prosthetic valve 100 within a host valvular structure 12, utilizing a delivery apparatus comprising a first balloon and a second balloon. The host valvular structure 12 may include and/or be any of a variety of structures, examples of which include a native valvular structure 29 of a native heart valve 20 or a valvular structure 113 of a previously implanted prosthetic valve. As shown in FIG. 36, the method comprises advancing, at 810, a delivery assembly to the host valvular structure. The delivery assembly comprises the prosthetic valve (i.e., the guest prosthetic valve 100) disposed over a component of a delivery apparatus, such as over a balloon catheter or a balloon of the delivery apparatus.

The delivery apparatus includes a first balloon and a second balloon. The first balloon is configured to expand a pilot puncture formed in a host leaflet, so as to form a leaflet opening, while the second balloon is configured to expand a prosthetic valve within the host valvular structure, subsequent to formation of at least one leaflet opening within at least one host leaflet of the host valvular structure. In some examples, the first balloon is a hole dilation balloon 650 and the second balloon is a valve expansion balloon 664 of a delivery apparatus 602. In some examples, the first balloon is an inner balloon 750 and the second balloon is an outer balloon 764 of a delivery apparatus 702.

The delivery assembly further comprises a perforating member 630, 730, which can be axially movable relative to the first balloon. In some examples, advancing the delivery assembly at 810 comprises approximating the perforating member 630, 730, or at least a distal end portion thereof 632, 732, to a target host leaflet 10 of the host valvular structure 12.

The method additionally comprises forming, at 820, a leaflet opening 52 within the host leaflet 10. The forming the leaflet opening 52 is performed with the perforating member 630, 730 and the first balloon 650, 750. The forming the leaflet opening at 820 can include forming, at 822, a pilot puncture 50. The forming the pilot puncture is performed by a perforating member 630, 730, which can be, in some examples, in the form of a needle having a distal angled surface 634, 734, a guidewire 638, 738 having a guidewire tip 639, 739 configured to be sharp enough and/or deliver RF energy so as to pierce the host leaflet 10, as well as any combination thereof. The forming the pilot puncture 822 can comprise distally advancing the perforating member 630, 730 through the host leaflet 10. In some examples, the perforating member 630, 730 can be retracted from the leaflet 10 at any stage subsequent to formation of the pilot puncture 50.

The forming the leaflet opening at 820 can additionally include positioning, at 824, the first balloon 650, 750, in a deflated state thereof, inside the pilot puncture 50. The forming the leaflet opening at 820 can additionally include inflating, at 70830, the first balloon 650, 750 inside the pilot puncture, so as to expand the pilot puncture 50, thus forming the leaflet opening 52.

In some examples, a leaflet opening 52 is formed inside one of the host leaflet 10 of the host valvular structure 12. In some examples, it may be desirable to for leaflet openings 52 in at least two host leaflets 10 of the host valvular structure 12. In some examples, the method comprises checking, at 850, whether a leaflet opening 52 needs to be formed in another host leaflet. If formation of another leaflet opening 52 in another host leaflet is desired, the method can include retracting, at 852, the delivery assembly 600, 700 from the host leaflet in which a leaflet opening was formed, which can optionally be referred to as a first host leaflet, and positioning the delivery assembly 600, 700 adjacent a second host leaflet, followed by forming, at 820, a second leaflet opening, in a similar manner to that described above. In some examples, the method can include, prior to the retracting the delivery assembly at 852, deflating the first balloon.

The method additionally comprises inflating, at 860, the second balloon 664, 764 inside the valvular structure 12. This can be performed subsequent to forming a leaflet opening inside a single host leaflet, without performing any of the checking at 850 or retracting at 852, or after deciding that no additional leaflet opening is required at 850. The inflating the second balloon at 860 can include either inflating the second balloon between host leaflets 10 of the host valvular structure 12, or inflating the second balloon inside the leaflet opening 52. If more than one leaflet opening 52 was formed in more than one host leaflet 10, the second balloon can be inflated inside the latest formed leaflet opening.

In some examples, the method comprises, prior to inflating the second balloon at 860, positioning the second balloon inside the host valvular structure 12. In some examples, when the second balloon is a valve expansion balloon 664 of a tandem balloon arrangement, it can be distally advanced to position it inside the leaflet opening prior to inflating inside the leaflet opening. In some examples, when the first balloon is retracted away from the leaflet opening 52 after formation of the leaflet opening at 820, the second balloon can be distally advanced to position it inside the leaflet opening prior to inflating it at 860. In some examples, when a valve is to be expanded between the host leaflets, the delivery assembly, comprising both the first and second balloons, can be retracted from the host leaflet 10 in which a leaflet opening 52 was formed at 820, and the second balloon can be navigated and positioned between the host leaflet prior to inflating it between the host leaflets.

The inflating the second balloon at 860 is performed while the prosthetic valve 100 is positioned around the second balloon, such that the prosthetic valve 100 is expanded within the host valvular structure 12 when the second balloon is inflated. In some examples, the prosthetic valve 100 is crimped over the second balloon 664, 764 prior to the advancing the delivery assembly at 810. In some examples, the prosthetic valve 100 positioned proximal to the second balloon during the advancing the delivery assembly at 810, in which case a push shaft 628, 728 of the delivery apparatus can be optionally utilized advance and position the prosthetic valve 100 around the second balloon, prior to inflation of the second balloon.

The method can additionally comprise retracting, at 880, the delivery apparatus from the patient, leaving the prosthetic valve 100 implanted against the host valvular structure. The retracting 880 can comprise deflating, at 882, the second balloon, prior to pulling the delivery apparatus away from the implanted prosthetic valve.

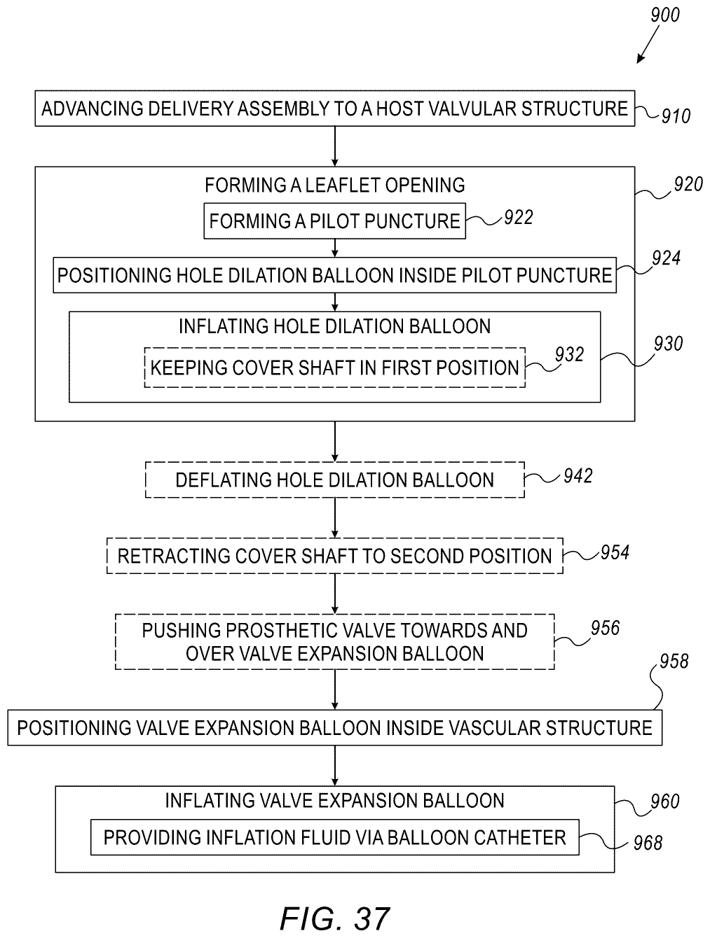

FIG. 37 is a flow chart depicting examples of a method 900 of implanting a prosthetic valve within a host valvular structure, utilizing a delivery apparatus comprising a hole dilation balloon 650 and a valve expansion balloon 664 proximal to the hole dilation balloon 650. The method 900 is an example of method 800, implemented with a delivery assembly 600 that includes a tandem balloon arrangement. The method 900 comprises advancing, at 910, the delivery assembly 600, in a similar manner to that described for the advancing at 810 with respect to FIG. 36. The method additionally comprises forming, at 920, a leaflet opening 52, which can include forming, at 922, a pilot puncture 50; positioning, at 924, the hole dilation balloon 650 inside the pilot puncture 50; and inflating, at 930, the hole dilation balloon 650, in a similar manner to that described for the forming the leaflet opening at 820, forming the pilot puncture at 822, positioning the first balloon at 824, and inflating the hole dilation balloon at 70830, respectively, and for the sake of brevity will not be described further.

While not explicitly shown in FIG. 37, it is to be understood that the method 900 can include stages equivalent to checking whether formation of another leaflet opening is required at 850 and if so, retracting and repositioning the delivery assembly at 852. The method 900 additionally comprises inflating, at 960, the valve expansion balloon 664 inside the host valvular structure 12, which can be performed in a similar manner to that described for the inflating the second balloon at 860, and can include inflating either between the host leaflets or inside a leaflet opening, as described with respect to FIG. 36. Furthermore, while not explicitly shown in FIG. 37, it is to be understood that the method 900 can include, subsequent to inflating the valve expansion balloon, retracting the delivery apparatus 602, in a similar manner described above with respect to the retracting at 880 and/or deflating the second balloon at 882.

In some examples, the delivery apparatus 602 can include a cover shaft movable between a first position and a second position, wherein a distal end of the cover shaft 624 is disposed around the valve expansion balloon 664 when the cover shaft 624 is in the first position, and wherein the distal end of the cover shaft 624 is proximal to the valve expansion balloon 664 in the second position of the cover shaft 624. In some examples, the inflating the hole dilation balloon 650 at 930 an optional step of keeping the cover shaft, at 932, in the first position during such inflation, so as to prevent simultaneous expansion of the valve expansion balloon 664 while the hole dilation balloon is inflated. In some examples, the distal portion of the cover shaft 624 is disposed between the valve expansion balloon 664 and the prosthetic valve 100 in the first position of the cover shaft, as described above and illustrated, for example, in FIG. 25A. In some examples, the distal portion of the cover shaft 624 is disposed around both the valve expansion balloon 664 and the prosthetic valve 100 in the first position of the cover shaft, as described above and illustrated, for example, in FIG. 26A.

In some examples, the inflating the hole dilation balloon 650 at 930 comprises providing inflation fluid via a balloon catheter 610 of the delivery apparatus 602. A lumen 612 of the balloon catheter 610 can be in fluid communication with the valve expansion balloon, while the valve expansion balloon 664 can be in fluid communication with the hole dilation balloon 650, such that inflation fluid can flow through the balloon catheter lumen 612 into a cavity 666 of the valve expansion balloon 664, and therefrom, into a cavity 654 of the hole dilation balloon 650, so as to inflate the hole dilation balloon 650.

While a cover shaft 624 can be utilized, as described above, to retain the valve expansion balloon in a deflated state while inflating the hole dilation balloon 650, in some examples, inflating balloon 650 while keeping balloon 664 deflated can achieved by controlling the pressure and/or volume of the inflation fluid. In some examples, providing inflation fluid via the balloon catheter comprises providing inflation fluid volume which is less than a volume threshold, set to be sufficient to expand the hole dilation balloon to a maximum diameter D1, but insufficient to expand the valve expansion balloon to a diameter that is greater than D1. In some examples, providing inflation fluid via the balloon catheter comprises providing inflation fluid at a pressure which is less than a pressure threshold, set to be sufficient to expand the hole dilation balloon to a maximum diameter D1, but insufficient to expand the valve expansion balloon to a diameter that is greater than D1.

In some examples, the hole dilation balloon 650 and the valve expansion balloon 664 are fluidly sealed from each other, each configured to be fed via a lumen of a different shaft or catheter of the delivery apparatus. In some examples, the inflating the hole dilation balloon 650 at 930 comprises providing inflation fluid via a nosecone shaft 616. A lumen 618 of the nosecone shaft 616 can be in fluid communication with the hole dilation balloon 650, while the balloon catheter lumen 612 can be in fluid communication with the valve expansion balloon, allowing each balloon to be independently inflated.

In some examples, the method 900 includes an optional step of deflating, at 942, the hole dilation balloon 650. This This can be performed, for example, to allow easier repositioning of the delivery assembly 600 after formation of the leaflet opening 52. In some examples, such as when a cover shaft 624 was optionally kept in the first position at 932, the method 900 can further include an optional step of retracting, at 954, the cover shaft 954, moving it to the second position. This will expose the valve expansion balloon 664, optionally allowing inflation thereof at 960.

As mentioned above, the prosthetic valve 100 can be positioned, during the advancing at 910, proximal to the valve expansion balloon 664. In such examples, the method 900 can include an optional step of pushing, at 956, the prosthetic valve 100 towards and over the valve expansion balloon 956, optionally by utilizing a push shaft 628 in a similar manner to that described above with respect to method 800.

Method 900 additionally comprises positioning, at 958, the valve expansion balloon 664 inside the valvular structure 12. Depending on whether valve expansion is desired between the host leaflets 10 or inside the leaflet opening 52, the valve expansion balloon 664 can be appropriately positioned, in a similar manner to that described above with respect to method 800.

It is to be understood that any order between the deflating at 942, retracting at 954, pushing at 956, and positioning at 958, is contemplated, and that any of the deflating at 942, retracting at 954, and/or pushing at 956, are merely optional. For example, when method 900 includes pushing the prosthetic valve at 956, it can be performed before or after any of the deflating at 942, the retracting at 954, and/or the positioning at 958. Moreover, it can be performed before, after, or during the forming the leaflet opening at 920. Furthermore, when the method 900 includes pushing the prosthetic valve at 956 prior to positioning the valve expansion balloon inside the valvular structure at 958, the push shaft 628 can be retained in position, pressed against an outflow end 106 of the prosthetic valve 100 during advancement of the valve expansion balloon 664, such as into the leaflet opening 52, so as to provide a counterforce that prevents the prosthetic valve from proximally slipping away from the balloon 664 during this maneuver.

The inflating the valve expansion balloon at 960 comprises providing, at 968, inflation fluid via the balloon catheter 610. In some examples, such as when both balloons 650, 664 are fluidly sealed and inflating the hole dilation balloon at 930 included providing inflation fluid via the nosecone shaft, the providing the inflation fluid via balloon catheter at 968 can separately inflate the valve expansion balloon 664, without affecting the hole dilation balloon.

In some examples, such as when the inflating the hole dilation balloon at 930 included providing inflation fluid volume which is less than a volume threshold, the providing inflation fluid via the balloon catheter at 968 comprises providing inflation fluid volume which is greater than or equal to the volume threshold, sufficient to expand the valve expansion balloon and the prosthetic valve disposed therearound to a diameter that is greater than D1.

In some examples, such as when the inflating the hole dilation balloon at 930 included providing inflation fluid pressure which is less than a pressure threshold, the providing inflation fluid via the balloon catheter at 968 comprises providing inflation fluid pressure which is greater than or equal to the pressure threshold, sufficient to expand the valve expansion balloon and the prosthetic valve disposed therearound to a diameter that is greater than D1.

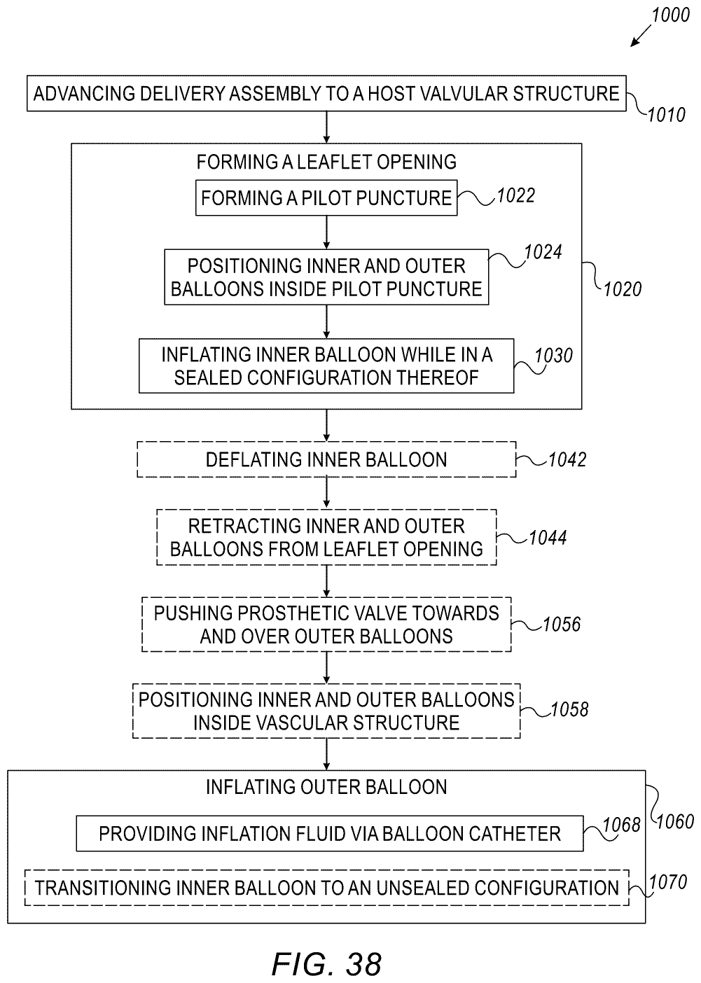

FIG. 38 is a flow chart depicting examples of a method 1000 of implanting a prosthetic valve within a host valvular structure, utilizing a delivery apparatus comprising an inner balloon 750 and an outer balloon 764 disposed around the inner balloon 750. The method 1000 is an example of method 800, implemented with a delivery apparatus 700 that includes a balloon-in-balloon arrangement. The method 1000 comprises advancing, at 1010, the delivery assembly 700, in a similar manner to that described for the advancing at 810 with respect to FIG. 36. The method additionally comprises forming, at 1020, a leaflet opening 52, which can include forming, at 1022, a pilot puncture 50; positioning, at 1024, the inner balloon 750 (along with the outer balloon 764 disposed therearound) inside the pilot puncture 50; and inflating, at 1030, the inner balloon 750, in a similar manner to that described for the forming the leaflet opening at 820, forming the pilot puncture at 822, positioning the first balloon at 824, and inflating the hole dilation balloon at 70830, respectively, and for the sake of brevity will not be described further.

While not explicitly shown in FIG. 38, it is to be understood that the method 1000 can include stages equivalent to checking whether formation of another leaflet opening is required at 850 and if so, retracting and repositioning the delivery assembly at 852. The method 1000 additionally comprises inflating, at 1060, the outer balloon 764 inside the host valvular structure 12, which can be performed in a similar manner to that described for the inflating the second balloon at 860, and can include inflating either between the host leaflets or inside a leaflet opening, as described with respect to FIG. 36. Furthermore, while not explicitly shown in FIG. 38, it is to be understood that the method 1000 can include, subsequent to inflating the outer balloon, retracting the delivery apparatus 702, in a similar manner described above with respect to the retracting at 880 and/or deflating the second balloon at 882.

The inner balloon 750 is provided, during the advancing at 1010, in a sealed configuration, such that it is fluidly sealed from the portion of the cavity 666 of outer balloon 664 disposed between the inner balloon wall 752 and the outer balloon wall 772. During the inflating the inner balloon 1030, inner balloon 750 is kept in this sealed configuration, such that it is inflated up to a maximum diameter D1, pushing against the outer balloon wall 273 to similarly expand the outer balloon 764 therewith, so as to expand the pilot puncture 50 to form the leaflet opening 52.

In some examples, the inflating the inner balloon 750 at 1030 comprises providing, at 634, inflation fluid via a balloon catheter 710 of the delivery apparatus 702. A lumen 712 of the balloon catheter 710 can be in fluid communication with the inner balloon, and the inner balloon 750 can be configured to transition to an unsealed configuration at a certain condition. In some examples, the inner balloon 750 is made of, or comprises, a weakened wall or region, such as a thinned wall or region. In such examples, providing inflation fluid via the balloon catheter can comprise providing inflation fluid at a pressure which is less than a pressure threshold, set to retain the inner balloon 750 in the sealed configuration, without tearing its wall. In some examples, the inner balloon 750 comprises a tear-line 746 which is kept intact during the inflating the inner balloon at 1030. In some examples, the inner balloon 750 comprises a flexible port 782 which is kept folded over itself to keep the inner balloon in the sealed configuration during the inflating the inner balloon at 1030.

In some examples, the inner balloon 750 can remain in a sealed configuration during the entire implantation procedure, wherein each of the inner and outer balloons is configured to be fed via a lumen of a different shaft or catheter of the delivery apparatus. In some examples, the inflating the inner balloon 750 at 1030 comprises providing inflation fluid via a nosecone shaft 716. A lumen 718 of the nosecone shaft 716 can be in fluid communication with the inner balloon 750, while the balloon catheter lumen 712 can be in fluid communication with the outer balloon 764, allowing each balloon to be independently inflated.

As mentioned above, the prosthetic valve 100 can be positioned, during the advancing at 1010, proximal to the outer balloon 764. In such examples, the method 1000 includes an optional step of pushing, at 1056, the prosthetic valve 100 towards and over the outer balloon 1056, optionally by utilizing a push shaft 728 in a similar manner to that described above with respect to method 800. In some examples, the method 1000 includes an optional step of retracting, at 1044, the inner balloon 750 (along with the outer balloon 764) from the leaflet opening 52. This can be performed, for example, to allow easier pushing of the prosthetic valve over the outer balloon at 1056. In some examples, the method 1000 includes an optional step of deflating, at 1042, the inner balloon 750. This can be performed, for example, to allow easier retraction of the inner balloon at 1044.

In some examples, method 1000 includes an optional step of positioning, at 1058, the outer balloon 764 (with the inner balloon 750 disposed therein) inside the valvular structure 12. This can be performed if the inner balloon 750 (with outer balloon 764) was retracted at 1044, and/or if prosthetic valve expansion is desired between the host leaflets 10.

It is to be understood that any order between the deflating at 1042, retracting at 654, pushing at 1056, and positioning at 1058, is contemplated, and that any of these stages is merely optional. For example, when method 1000 includes pushing the prosthetic valve at 1056, it can be performed before or after any of the deflating at 1042, the retracting at 654, and/or the positioning at 1058. Moreover, it can be performed before, after, or during the forming the leaflet opening at 1020. Furthermore, when the method 1000 includes pushing the prosthetic valve at 1056 subsequent to the retracting at 1044 and prior to positioning the outer balloon inside the valvular structure at 1058, the push shaft 728 can be retained in position, pressed against an outflow end 106 of the prosthetic valve 100 during advancement of the outer balloon 764, such as into the leaflet opening 52, so as to provide a counterforce that prevents the prosthetic valve from proximally slipping away from the balloon 764 during this maneuver.

The inflating the valve expansion balloon at 1060 comprises providing, at 1068, inflation fluid via the balloon catheter 1010. In some examples, such as when inflating the inner balloon at 1030 included providing inflation fluid via the nosecone shaft, the providing the inflation fluid via balloon catheter at 1068 can separately inflate the outer balloon 764, without affecting the inner balloon.

In some examples, the inflating the outer balloon at 1060 includes an optional step of transitioning, at 1070, the inner balloon 750 to the unsealed configuration, such that inflation fluid can freely flow from balloon catheter lumen 712, via inner balloon 750, into the cavity 766 of outer balloon 764 to inflate the outer balloon and expand the prosthetic valve. In some examples, such as when the inflating the inner at 1030 included providing inflation fluid pressure which is less than a pressure threshold, the transitioning to the unsealed configuration at 1070 comprises providing, inflation fluid pressure which is greater than or equal to the pressure threshold, sufficient to tear the inner balloon wall 752 or a weakened region thereof. In some examples, such as when the inner balloon 750 comprises a tear-line 746, the transitioning to the unsealed configuration at 1070 comprises tearing, the tear-line 746, by proximally pulling a ripcord 740 attached at a distal end 742 thereof to the tear-line 746. In some examples, such as when the inner balloon 750 comprises a pre-folded flexible port 780, the transitioning to the unsealed configuration at 1070 comprises unfolding, the flexible port 780, by proximally pulling a release cord 786 equipped with a loop 785 disposed around a port fold 782 of the flexible port 780.

Any of the systems, devices, apparatuses, etc. herein can be sterilized (for example, with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Technology

Some examples of the above-described technology are enumerated below. It should be noted that one feature of an example in isolation or more than one feature of the example taken in combination and, optionally, in combination with one or more features of one or more further examples are further examples also falling within the disclosure of this application.

Example 1. A leaflet perforation tool, comprising: a catheter comprising a distal end portion configured to be inserted into a patient's vasculature, wherein the catheter comprises a lumen; a lacerating member disposed within the lumen of the catheter, wherein the lacerating member is configured to pierce a leaflet of a host valvular structure to form a pilot puncture in the leaflet; and an expansion member supported by the distal end portion of the catheter, wherein the expansion member is configured to be inserted within the pilot puncture and to be selectively transitioned between a radially compressed configuration and a radially expanded configuration; wherein, when the expansion member is received within the pilot puncture, transitioning the expansion member from the radially compressed configuration to the radially expanded configuration expands the pilot puncture to form a leaflet opening.

Example 2. The leaflet perforation tool of any example herein, particularly example 1, wherein the host valvular structure is a valvular structure of a native heart valve.

Example 3. The leaflet perforation tool of any example herein, particularly example 1, wherein the host valvular structure is a valvular structure of a previously implanted prosthetic valve that is implanted within a native heart valve.

Example 4. The leaflet perforation tool of any example herein, particularly any one of examples 1-3, wherein the lacerating member comprises a distal end portion that is configured to be selectively translated in a proximal direction and in a distal direction relative to the distal end portion of the catheter.

Example 5. The leaflet perforation tool of any example herein, particularly example 4, wherein the distal end portion of the lacerating member terminates at an angled surface.

Example 6. The leaflet perforation tool of any example herein, particularly example 5, wherein the distal end portion of the catheter terminates at an angled surface, and wherein, when the distal end portion of the lacerating member is axially aligned with the distal end portion of the catheter, the angled surface of the lacerating member is at least substantially coplanar with the angled surface of the catheter.

Example 7. The leaflet perforation tool of any example herein, particularly any one of examples 1-6, wherein the lacerating member comprises a needle.

Example 8. The leaflet perforation tool of any example herein, particularly example 7, wherein the needle is one or both of a spring-loaded needle and a Veress needle.

Example 9. The leaflet perforation tool of any example herein, particularly any one of examples 1-8, wherein the lacerating member comprises a lumen that is configured to receive a perforation tool guidewire of the leaflet perforation tool.

Example 10. The leaflet perforation tool of any example herein, particularly any one of examples 1-6, wherein the lacerating member comprises a guidewire, and optionally an RF energy source coupled to the guidewire that provides RF energy to a tip of the guidewire.

Example 11. The leaflet perforation tool of any example herein, particularly any one of examples 1-10, wherein one or both of the catheter and the lacerating member is curved to facilitate positioning the catheter and the lacerating member relative to the leaflet.

Example 12. The leaflet perforation tool of any example herein, particularly any one of examples 1-11, wherein the expansion member extends at least substantially around a circumference of the catheter.

Example 13. The leaflet perforation tool of any example herein, particularly any one of examples 1-12, wherein the expansion member has a circular profile when in the radially expanded configuration.

Example 14. The leaflet perforation tool of any example herein, particularly any one of examples 1-13, wherein the expansion member comprises an inflatable balloon that is configured to be selectively inflated and deflated to transition the expansion member between the radially compressed configuration and the radially expanded configuration.

Example 15. The leaflet perforation tool of any example herein, particularly example 14, wherein the radially compressed configuration is a deflated state of the inflatable balloon, and wherein the radially expanded configuration is an inflated state of the inflatable balloon.

Example 16. The leaflet perforation tool of any example herein, particularly any one of examples 1-13, wherein the expansion member is configured to permit blood to flow therethrough.

Example 17. The leaflet perforation tool of any example herein, particularly any one of examples 1-13 or 16, wherein the expansion member comprises an expandable frame comprising a collapsible support structure that is radially expandable and compressible to transition the expandable frame between the radially compressed configuration and the radially expanded configuration.

Example 18. The leaflet perforation tool of any example herein, particularly example 17, wherein the collapsible support structure comprises a plurality of frame members arranged and connected such that the collapsible support structure expands radially as the collapsible support structure is compressed axially.

Example 19. The leaflet perforation tool of any example herein, particularly example 18, wherein the frame members define one or more diamond shaped cells such that the collapsible support structure expands radially when the diamond shaped cells are compressed axially.

Example 20. The leaflet perforation tool of any example herein, particularly any one of examples 17-19, wherein the expandable frame comprises a distal end portion and a proximal end portion that are operatively coupled to one another via a screw mechanism, wherein rotation of the screw mechanism in a first direction causes a distance between the distal end portion and the proximal end portion to decrease and produce radial expansion of the expandable frame, and wherein rotation of the screw mechanism is a second direction causes a distance between the distal end portion and the proximal end portion to increase and produce radial compression of the expandable frame.

Example 21. The leaflet perforation tool of any example herein, particularly any one of examples 1-20, further comprising one or more positioning arms supported by the catheter and configured to facilitate positioning the catheter relative to the host valvular structure.

Example 22. The leaflet perforation tool of any example herein, particularly example 21, wherein at least one positioning arm of the one or more positioning arms is configured to engage a pocket formed along a cusp edge of the leaflet.

Example 23. The leaflet perforation tool of any example herein, particularly any one of examples 21-22, wherein at least one positioning arm of the one or more positioning arms is configured to engage a free edge of the leaflet.

Example 24. The leaflet perforation tool of any example herein, particularly any one of examples 1-23, wherein the leaflet perforation tool is sterilized.

Example 25. A leaflet perforation tool, comprising: a catheter comprising a distal end portion configured to be inserted into a patient's vasculature; and a nosecone coupled to the distal end portion of the catheter; wherein the nosecone comprises a main body with an external surface and at least one blade coupled to the main body, and wherein the nosecone is configured to be inserted through and form a leaflet opening in a leaflet of a host valvular structure.

Example 26. The leaflet perforation tool of any example herein, particularly example 25, wherein the host valvular structure is a valvular structure of a native heart valve.

Example 27. The leaflet perforation tool of any example herein, particularly example 25, wherein the host valvular structure is a valvular structure of a previously implanted prosthetic valve that is implanted within a native heart valve.

Example 28. The leaflet perforation tool of any example herein, particularly any one of examples 25-27, wherein each blade of the at least one blade is positioned fully radially inward of the external surface.

Example 29. The leaflet perforation tool of any example herein, particularly any one of examples 25-28, wherein, for each blade of the at least one blade, the blade comprises a blade edge that is recessed relative to the external surface.

Example 30. The leaflet perforation tool of any example herein, particularly any one of examples 25-29, wherein the nosecone extends along a central axis, and wherein each blade of the at least one blade comprises a respective blade edge that extends along a direction that is at least substantially coplanar with the central axis.

Example 31. The leaflet perforation tool of any example herein, particularly example 30, wherein, for each blade of the at least one blade, the blade edge extends along a direction that is at least substantially parallel to the central axis.

Example 32. The leaflet perforation tool of any example herein, particularly any one of examples 25-31, wherein the nosecone comprises at least one groove that is recessed relative to the external surface of the main body, and wherein the at least one blade comprises a first blade that is positioned within a first groove of the at least one groove.

Example 33. The leaflet perforation tool of any example herein, particularly example 32, wherein each groove of the at least one groove extends at least partially circumferentially around the nosecone.

Example 34. The leaflet perforation tool of any example herein, particularly any one of examples 32-33, wherein each groove of the at least one groove extends only partially circumferentially around the nosecone.

Example 35. The leaflet perforation tool of any example herein, particularly any one of examples 32-33, wherein each groove of the at least one groove extends fully circumferentially around the nosecone.

Example 36. The leaflet perforation tool of any example herein, particularly any one of examples 32-35, wherein the at least one blade further comprises a second blade positioned within the first groove.

Example 37. The leaflet perforation tool of any example herein, particularly example 36, wherein the first blade and the second blade are at least partially axially aligned with one another.

Example 38. The leaflet perforation tool of any example herein, particularly any one of examples 36-37, wherein the first blade and the second blade are circumferentially offset from one another by an angle that is about 180 degrees.

Example 39. The leaflet perforation tool of any example herein, particularly any one of examples 25-31, wherein the nosecone comprises a plurality of grooves that are axially offset from one another, and wherein each groove of the plurality of grooves receives a respective first blade of the at least one blade and a respective second blade of the at least one blade.

Example 40. The leaflet perforation tool of any example herein, particularly any one of examples 25-31, wherein the nosecone comprises a plurality of grooves that are at least partially axially offset from one another, wherein the at least one blade comprises a plurality of blades, and wherein each blade of the plurality of blades is received within a respective groove of the plurality of grooves.

Example 41. The leaflet perforation tool of any example herein, particularly example 40, wherein the plurality of grooves are positioned such that, for each blade of the plurality of blades, at least a portion of the blade is axially offset from each other blade of the plurality of blades.

Example 42. The leaflet perforation tool of any example herein, particularly any one of examples 40-41, wherein two grooves of the plurality of grooves are positioned such that the respective blades positioned within the two grooves are circumferentially offset from one another by an angle that is about 180 degrees.

Example 43. The leaflet perforation tool of any example herein, particularly any one of examples 25-42, wherein the nosecone comprises a lumen that is configured to receive a perforation tool guidewire of the leaflet perforation tool.

Example 44. The leaflet perforation tool of any example herein, particularly any one of examples 25-43, wherein at least a portion of the nosecone is substantially conical in shape.

Example 45. The leaflet perforation tool of any example herein, particularly any one of examples 25-44, wherein the nosecone comprises a distal portion and a proximal portion that have different shapes.

Example 46. The leaflet perforation tool of any example herein, particularly example 45, wherein the distal portion is at least substantially conical in shape, and wherein the proximal portion is at least substantially frustoconical in shape.

Example 47. The leaflet perforation tool of any example herein, particularly any one of examples 25-46, further comprising a lacerating member configured to pierce the leaflet to form a pilot puncture in the leaflet, and wherein the nosecone is configured to extend within the pilot puncture and to expand the pilot puncture to form the leaflet opening.

Example 48. The leaflet perforation tool of any example herein, particularly example 47, wherein the lacerating member comprises a needle.

Example 49. The leaflet perforation tool of any example herein, particularly example 48, wherein the needle is one or both of a spring-loaded needle and a Veress needle.

Example 50. The leaflet perforation tool of any example herein, particularly any one of examples 47-49, wherein the nosecone comprises a lumen, wherein the lacerating member is disposed within the lumen of the nosecone, and wherein the lacerating member comprises a distal end that is configured to be selectively translated in a proximal direction and in a distal direction relative to a distal end of the nosecone.

Example 51. The leaflet perforation tool of any example herein, particularly any one of examples 47-50, wherein the lacerating member comprises a lumen that is configured to receive a perforation tool guidewire of the leaflet perforation tool.

Example 52. The leaflet perforation tool of any example herein, particularly example 47, wherein the lacerating member comprises a guidewire, and optionally an RF energy source coupled to the guidewire that provides RF energy to a tip of the guidewire.

Example 53. The leaflet perforation tool of any example herein, particularly any one of examples 25-52, wherein the leaflet perforation tool is sterilized.

Example 54. A leaflet perforation tool, comprising: a catheter comprising a distal end portion configured to be inserted into a patient's vasculature; and a nosecone coupled to the distal end portion of the catheter; wherein the nosecone comprises a main body with an external surface and at least one blade recessed within the main body, and wherein the leaflet perforation tool is configured such that, when the nosecone is inserted within a leaflet of a host valvular structure, the nosecone forms a leaflet opening within the leaflet and the blade forms a leaflet slit that is connected to the leaflet opening.

Example 55. The leaflet perforation tool of any example herein, particularly example 54, wherein each blade of the at least one blade is positioned fully radially inward of the external surface.

Example 56. The leaflet perforation tool of any example herein, particularly any one of examples 54-55, wherein each blade of the at least one blade comprises a respective blade edge that is recessed relative to the external surface.

Example 57. The leaflet perforation tool of any example herein, particularly any one of examples 54-56, wherein the nosecone comprises at least one groove that is recessed relative to the external surface and that extends fully circumferentially around the nosecone, and wherein the at least one blade comprises a first blade and a second blade positioned within a first groove of the at least one groove.

Example 58. The leaflet perforation tool of any example herein, particularly example 57, wherein the first blade and the second blade are axially aligned with one another.

Example 59. The leaflet perforation tool of any example herein, particularly any one of examples 54-56, wherein the nosecone comprises a plurality of grooves that are recessed relative to the external surface of the main body, wherein the plurality of grooves are axially offset from one another, wherein the at least one blade comprises a plurality of blades, and wherein each blade of the plurality of blades is received within a respective groove of the plurality of grooves.

Example 60. The leaflet perforation tool of any example herein, particularly example 59, wherein the plurality of grooves are positioned such that, for each blade of the plurality of blades, at least a portion of the blade is axially offset from each other blade of the plurality of blades.

Example 61. The leaflet perforation tool of any example herein, particularly any one of examples 54-60, further comprising a lacerating member configured to pierce the leaflet to form a pilot puncture in the leaflet, and wherein the nosecone is configured to extend within the pilot puncture and to expand the pilot puncture to form the leaflet opening.

Example 62. The leaflet perforation tool of any example herein, particularly example 61, wherein the lacerating member comprises a needle.

Example 63. The leaflet perforation tool of any example herein, particularly example 62, wherein the needle is one or both of a spring-loaded needle and a Veress needle.

Example 64. The leaflet perforation tool of any example herein, particularly any one of examples 61-63, wherein the nosecone comprises a lumen, wherein the lacerating member is disposed within the lumen of the nosecone, and wherein the lacerating member comprises a distal end that is configured to be selectively translated in a proximal direction and in a distal direction relative to a distal end of the nosecone.

Example 65. The leaflet perforation tool of any example herein, particularly any one of examples 61-64, wherein the lacerating member comprises a lumen that is configured to receive a perforation tool guidewire of the leaflet perforation tool.

Example 66. The leaflet perforation tool of any example herein, particularly example 61, wherein the lacerating member comprises a guidewire.

Example 67. The leaflet perforation tool of any example herein, particularly any one of examples 54-66, wherein the leaflet perforation tool is sterilized.

Example 68. A method of implanting a replacement prosthetic valve within a host valvular structure, the method comprising: forming, with a leaflet perforation tool, a leaflet opening within a leaflet of a host valvular structure; positioning a replacement prosthetic valve in a radially compressed configuration within the leaflet opening; and radially expanding the replacement prosthetic valve.

Example 69. The method of any example herein, particularly example 68, further comprising, prior to the forming the leaflet opening, positioning a distal end portion of the leaflet perforation tool adjacent the leaflet.

Example 70. The method of any example herein, particularly example 69, wherein the leaflet perforation tool comprises a catheter and a positioning arm supported by the catheter, and wherein the positioning the leaflet perforation tool relative to the leaflet comprises positioning the catheter such that the positioning arm engages a pocket formed along a cusp edge of the leaflet.

Example 71. The method of any example herein, particularly example 69, wherein the leaflet perforation tool comprises a catheter and a positioning arm supported by the catheter, and wherein the positioning the leaflet perforation tool relative to the leaflet comprises positioning the catheter such that the positioning arm engages a free edge of the leaflet.

Example 72. The method of any example herein, particularly any one of examples 69-71, wherein the positioning the leaflet perforation tool relative to the leaflet comprises advancing the leaflet perforation tool toward the leaflet over a perforation tool guidewire.

Example 73. The method of any example herein, particularly any one of examples 68-69, wherein the forming the leaflet opening comprises: forming, with the leaflet perforation tool, a pilot puncture within the leaflet; inserting a portion of the leaflet perforation tool into the pilot puncture; and expanding the pilot puncture to form the leaflet opening.

Example 74. The method of any example herein, particularly example 73, wherein the leaflet perforation tool comprises a catheter and a lacerating member disposed within a lumen of the catheter, and wherein the forming the pilot puncture within the leaflet comprises translating the lacerating member in a distal direction relative to the catheter to pierce the leaflet to form the pilot puncture.

Example 75. The method of any example herein, particularly any one of examples 68-69, wherein the leaflet perforation tool comprises a catheter and an expansion member supported by the catheter, and wherein the forming the leaflet opening comprises: forming, with the leaflet perforation tool, a pilot puncture within the leaflet; positioning the expansion member within the pilot puncture; and transitioning the expansion member from a radially compressed configuration to a radially expanded configuration to expand the pilot puncture into the leaflet opening.

Example 76. The method of any example herein, particularly example 75, wherein the leaflet perforation tool comprises a lacerating member disposed within a lumen of the catheter, wherein the forming the pilot puncture within the leaflet comprises translating the lacerating member in a distal direction relative to the catheter to pierce the leaflet to form the pilot puncture, and where the method further comprises, subsequent to the forming the pilot puncture and prior to the transitioning the expansion member from the radially compressed configuration to the radially expanded configuration, retracting the lacerating member away from the leaflet.

Example 77. The method of any example herein, particularly any one of examples 75-76, wherein the expansion member comprises an inflatable balloon, and wherein the transitioning the expansion member from the radially compressed configuration to the radially expanded configuration comprises inflating the inflatable balloon to transition the inflatable balloon from a deflated state to an inflated state.

Example 78. The method of any example herein, particularly any one of examples 75-76, wherein the expansion member comprises an expandable frame, and wherein the transitioning the expansion member from the radially compressed configuration to the radially expanded configuration comprises mechanically expanding the expandable frame.

Example 79. The method of any example herein, particularly any one of examples 68-69, wherein the leaflet perforation tool comprises a catheter and a nosecone coupled to a distal end of the catheter, wherein the nosecone comprises a main body and at least one blade coupled to the main body, and wherein the forming the leaflet opening comprises inserting the nosecone into the leaflet such that the nosecone forms the leaflet opening within the leaflet and such that the at least one blade forms a leaflet slit that is connected to the leaflet opening.

Example 80. The method of any example herein, particularly example 79, wherein the at least one blade comprises a plurality of blades, and wherein the inserting the nosecone into the leaflet comprises advancing the nosecone through the leaflet such that each blade of the plurality of blades forms a respective leaflet slit.

Example 81. The method of any example herein, particularly any one of examples 79-80, wherein the forming the leaflet opening comprises, prior to the inserting the leaflet perforation tool into the leaflet, forming a pilot puncture in the leaflet with a lacerating member of the leaflet perforation tool, and wherein the inserting the leaflet perforation tool into the leaflet comprises inserting the nosecone into the pilot puncture to expand the pilot puncture into the leaflet opening.

Example 82. The method of any example herein, particularly example 81, wherein the nosecone comprises a lumen, wherein the lacerating member is disposed within the lumen of the nosecone, and wherein the forming the pilot puncture in the leaflet comprises translating the lacerating member in a distal direction relative to the nosecone and through the leaflet to form the pilot puncture.

Example 83. The method of any example herein, particularly any one of examples 68-69, wherein the positioning the replacement prosthetic valve within the leaflet opening comprises advancing the replacement prosthetic valve into the leaflet opening via a replacement valve guidewire.

Example 84. The method of any example herein, particularly example 83, wherein the positioning the leaflet perforation tool relative to the leaflet comprises advancing the leaflet perforation tool toward the leaflet via the replacement valve guidewire.

Example 85. The method of any example herein, particularly any one of examples 83-84, wherein the positioning the leaflet perforation tool relative to the leaflet comprises advancing the leaflet perforation tool toward the leaflet via a perforation tool guidewire that extends alongside the replacement valve guidewire.

Example 86. The method of any example herein, particularly any one of examples 68-85, wherein the radially expanding the replacement prosthetic valve increases a size of the leaflet opening.

Example 87. The method of any example herein, particularly any one of examples 68-86, wherein the radially expanding the replacement prosthetic valve tears the leaflet.

Example 88. The method of any example herein, particularly any one of examples 68-87, wherein the radially expanding the replacement prosthetic valve modifies the leaflet such that the leaflet does not obstruct a frame opening in a frame of the replacement prosthetic valve.

Example 89. The method of any example herein, particularly any one of examples 68-88, wherein the radially expanding the replacement prosthetic valve moves the leaflet to a location upstream of a downstream edge of an outer skirt of the replacement prosthetic valve.

Example 90. The method of any example herein, particularly any one of examples 68-89, wherein the radially expanding the replacement prosthetic valve comprises inflating a valve delivery inflatable balloon.

Example 91. The method of any example herein, particularly any one of examples 68-90, wherein the radially expanding the replacement prosthetic valve comprises actuating a mechanical actuator of the replacement prosthetic valve.

Example 92. The method of any example herein, particularly any one of examples 68-69, wherein the leaflet is a first leaflet, wherein the leaflet opening is a first leaflet opening formed in the first leaflet, and wherein the method further comprises: forming a second leaflet opening within a second leaflet of the host valvular structure.

Example 93. The method of any example herein, particularly example 92, wherein the leaflet perforation tool comprises a catheter and an expansion member supported by the catheter, wherein the forming the first leaflet opening comprises: forming, with the leaflet perforation tool, a first pilot puncture within the first leaflet; positioning the expansion member within the first pilot puncture; and transitioning the expansion member from a radially compressed configuration to a radially expanded configuration to expand the first pilot puncture into the first leaflet opening; and wherein the forming the second leaflet opening comprises: forming, with the leaflet perforation tool, a second pilot puncture within the second leaflet; positioning the expansion member within the second pilot puncture; and transitioning the expansion member from the radially compressed configuration to the radially expanded configuration to expand the second pilot puncture into the second leaflet opening.

Example 94. The method of any example herein, particularly example 92, wherein the leaflet perforation tool further comprises a catheter and a lacerating member disposed within a lumen of the catheter, wherein the forming the second leaflet opening comprises forming a second pilot puncture within the second leaflet by translating the lacerating member in a distal direction relative to the catheter to pierce the second leaflet.

Example 95. The method of any example herein, particularly example 92, wherein the leaflet perforation tool comprises a catheter and a nosecone coupled to a distal end of the catheter, wherein the nosecone comprises a main body and at least one blade coupled to the main body, and wherein the forming the second leaflet opening comprises inserting the leaflet perforation tool into the second leaflet such that the nosecone forms the second leaflet opening within the second leaflet and such that the at least one blade forms a leaflet slit that is connected to the second leaflet opening.

Example 96. The method of any example herein, particularly example 92, further comprising: positioning the replacement prosthetic valve in the radially compressed configuration within the first leaflet opening; and radially expanding the replacement prosthetic valve within the first leaflet.

Example 97. The method of any example herein, particularly example 96, wherein the radially expanding the replacement prosthetic valve operates to modify the host valvular structure such that at least a portion of the second leaflet does not obstruct a frame opening in a frame of the replacement prosthetic valve.

Example 98. The method of any example herein, particularly example 96, wherein the radially expanding the replacement prosthetic valve operates to modify the host valvular structure such the second leaflet opening is aligned with a frame opening in a frame of the replacement prosthetic valve.

Example 99. A method comprising: forming a pilot puncture within a leaflet of a host valvular structure with a laceration member of a leaflet perforation tool; positioning an expansion member of the leaflet perforation tool within the pilot puncture; and transitioning the expansion member from a radially compressed configuration to a radially expanded configuration to expand the pilot puncture into the leaflet opening.

Example 100. The method of any example herein, particularly example 99, wherein the expansion member comprises an inflatable balloon, and wherein the transitioning the expansion member from the radially compressed configuration to the radially expanded configuration comprises inflating the inflatable balloon to transition the inflatable balloon from a deflated state to an inflated state.

Example 101. The method of any example herein, particularly any one of examples 99-100, wherein the expansion member comprises an expandable frame, and wherein the transitioning the expansion member from the radially compressed configuration to the radially expanded configuration comprises mechanically expanding the expandable frame.

Example 102. The method of any example herein, particularly any one of examples 99-101, further comprising: positioning a replacement prosthetic valve in a radially compressed configuration within the leaflet opening; and radially expanding the replacement prosthetic valve.

Example 103. The method of any example herein, particularly example 102, wherein the radially expanding the replacement prosthetic valve comprises inflating a valve delivery inflatable balloon.

Example 104. The method of any example herein, particularly any one of examples 102-103, wherein the radially expanding the replacement prosthetic valve comprises actuating a mechanical actuator of the replacement prosthetic valve.

Example 105. A method comprising: forming, with a leaflet perforation tool, a tear or opening within a leaflet of a host valvular structure; wherein the leaflet perforation tool comprises a catheter and a nosecone coupled to a distal end of the catheter, and wherein the nosecone comprises a main body and at least one blade coupled to the main body.

Example 106. The method of any example herein, particularly example 105, wherein the forming the tear or opening comprises inserting the nosecone into the leaflet such that the nosecone forms the tear or opening within the leaflet and such that the at least one blade forms a leaflet slit that is connected to the tear or opening.

Example 107. The method of any example herein, particularly example 105, wherein the at least one blade comprises a plurality of blades, and wherein the forming the tear or opening comprises inserting the nosecone into the leaflet and advancing the nosecone through the leaflet such that each blade of the plurality of blades forms a respective leaflet slit.

Example 108. The method of any example herein, particularly any one of examples 105-107, wherein the forming the tear or opening comprises, prior to the inserting the leaflet perforation tool into the leaflet, forming a pilot puncture in the leaflet with a lacerating member of the leaflet perforation tool, and wherein the inserting the leaflet perforation tool into the leaflet comprises inserting the nosecone into the pilot puncture to expand the pilot puncture into the tear or opening.

Example 109. The method of any example herein, particularly example 108, wherein the nosecone comprises a lumen, wherein the lacerating member is disposed within the lumen of the nosecone, and wherein the forming the pilot puncture in the leaflet comprises translating the lacerating member in a distal direction relative to the nosecone and through the leaflet to form the pilot puncture.

Example 110. The method of any example herein, particularly any one of examples 105-109, further comprising: positioning a replacement prosthetic valve in a radially compressed configuration within the tear or opening; and radially expanding the replacement prosthetic valve.

Example 111. The method of any example herein, particularly example 110, wherein the radially expanding the replacement prosthetic valve comprises inflating a valve delivery inflatable balloon.

Example 112. The method of any example herein, particularly any one of examples 110-111, wherein the radially expanding the replacement prosthetic valve comprises actuating a mechanical actuator of the replacement prosthetic valve.

Example 113. A delivery assembly, comprising:
a delivery apparatus comprising:
   a handle;
   a first balloon configured to transition between deflated and inflated states thereof;
   a second balloon configured to transition between deflated and inflated states thereof;
   a balloon catheter extending from the handle, the balloon catheter defining a balloon catheter lumen, wherein the balloon catheter lumen is in fluid communication with at least one of the first balloon and the second balloon;
   a perforating member configured to pierce a host leaflet of a host valvular structure to form a pilot puncture in the host leaflet; and
a prosthetic valve comprising a frame movable between a radially compressed and a radially expanded configuration;
wherein the first balloon is configured to be inserted within the pilot puncture.

Example 114. The delivery assembly of any example herein, particularly example 113, wherein the perforating member comprises a distal end portion configured to be positioned distal to the first balloon for formation of the pilot puncture.

Example 115. The delivery assembly of any example herein, particularly example 113 or 114, wherein the maximum diameter of the second balloon in its inflated state is greater than the maximum diameter of the first balloon in its inflated state.

Example 116. The delivery assembly of any example herein, particularly any one of examples 113 to 115, wherein inflation of the first balloon, while positioned within the pilot puncture, is configured to expand the pilot puncture to form a leaflet opening.

Example 117. The delivery assembly of any example herein, particularly any one of examples 113 to 116, wherein inflation of the second balloon, while positioned within the leaflet opening with the prosthetic valve disposed around the second balloon, is configured to expand the prosthetic valve to implant the prosthetic valve in the host valvular structure.

Example 118. The delivery assembly of any example herein, particularly example 114, wherein the distal end portion of the perforating member is axially movable relative to the first balloon.

Example 119. The delivery assembly of any example herein, particularly example 118, wherein the distal end portion of the perforating member terminates at an angled surface.

Example 120. The delivery assembly of any example herein, particularly example 118 or 119, wherein the perforating member comprises a needle.

Example 121. The delivery assembly of any example herein, particularly example 120, wherein the needle is one or both of a spring-loaded needle and a Veress needle.

Example 122. The delivery assembly of any example herein, particularly any one of examples 118 to 121, wherein the perforating member comprises a perforating member lumen.

Example 123. The delivery assembly of any example herein, particularly example 122, further comprising a guidewire extending through the perforating member lumen.

Example 124. The delivery assembly of any example herein, particularly example 123, wherein the guidewire comprises a sharp tip configured to penetrate through the host leaflet.

Example 125. The delivery assembly of any example herein, particularly example 123, further comprising an RF energy source coupled to the guidewire and configured to provide RF energy to a tip of the guidewire.

Example 126. The delivery assembly of any example herein, particularly any one of examples 113 to 117, wherein the perforating member is a guidewire extending through the balloon catheter.

Example 127. The delivery assembly of any example herein, particularly example 126, wherein the guidewire comprises a sharp tip configured to penetrate through the host leaflet.

Example 128. The delivery assembly of any example herein, particularly example 126, further comprising an RF energy source coupled to the guidewire and configured to provide RF energy to a tip of the guidewire.

Example 129. The delivery assembly of any example herein, particularly any one of examples 113 to 128, wherein the prosthetic valve is disposed around at least one of the balloon catheter and the second balloon.

Example 130. The delivery assembly of any example herein, particularly any one of examples 113 to 129, wherein the prosthetic valve and the second balloon are axially movable relative to each other.

Example 131. The delivery assembly of any example herein, particularly any one of examples 113 to 130, wherein the prosthetic valve is movable between a position proximal to the second balloon and a position around the second balloon.

Example 132. The delivery assembly of any example herein, particularly any one of examples 113 to 130, wherein the delivery apparatus further comprises a push shaft configured to push the prosthetic valve from a position proximal to the second balloon toward the second balloon.

Example 133. The delivery assembly of any example herein, particularly any one of examples 113 to 132, wherein the second balloon is a valve expansion balloon, and wherein the first balloon is a hole dilation balloon positioned distal to the valve expansion balloon.

Example 134. The delivery assembly of any example herein, particularly example 133, wherein the valve expansion balloon mounted on the balloon catheter and is in fluid communication with the balloon catheter lumen.

Example 135. The delivery assembly of any example herein, particularly example 134, wherein the hole dilation balloon comprises a dilation balloon cavity, and wherein the valve expansion balloon comprises an expansion balloon cavity.

Example 136. The delivery assembly of any example herein, particularly example 135, wherein the delivery apparatus further comprises a nosecone shaft extending through the balloon catheter lumen and defining a nosecone shaft lumen.

Example 137. The delivery assembly of any example herein, particularly example 136, wherein the delivery apparatus further comprises a nosecone attached to a distal end of the nosecone shaft.

Example 138. The delivery assembly of any example herein, particularly example 137, wherein the nosecone is distal to the hole dilation balloon.

Example 139. The delivery assembly of any example herein, particularly any one of examples 136 to 138, wherein the perforating member extends through the nosecone shaft.

Example 140. The delivery assembly of any example herein, particularly example 139, wherein the perforating member is axially movable relative to the nosecone shaft.

Example 141. The delivery assembly of any example herein, particularly any one of examples 135 to 140, wherein the hole dilation balloon is in fluid communication with the valve expansion balloon.

Example 142. The delivery assembly of any example herein, particularly example 141, wherein the delivery apparatus further comprises an intermediate connector disposed between the dilation balloon cavity and the expansion balloon cavity.

Example 143. The delivery assembly of any example herein, particularly example 142, wherein the intermediate connector comprises an intermediate channel in fluid communication with the dilation balloon cavity and the expansion balloon cavity.

Example 144. The delivery assembly of any example herein, particularly example 142 or 143, wherein the intermediate connector comprises a connector inner surface attached to the balloon catheter.

Example 145. The delivery assembly of any example herein, particularly any one of examples 136 to 140, wherein the hole dilation balloon is in fluid communication with the valve expansion balloon, and wherein the delivery apparatus further comprises an intermediate connector disposed between an internal cavity of the hole dilation balloon and an internal cavity of the valve expansion balloon, and wherein the intermediate connector comprises a connector inner surface attached to the nosecone shaft.

Example 146. The delivery assembly of any example herein, particularly any one of examples 142 to 145, wherein the intermediate connector comprises a connector outer surface attached to the hole dilation balloon.

Example 147. The delivery assembly of any example herein, particularly any one of examples 142 to 146, wherein the intermediate connector comprises a connector outer surface attached to the valve expansion balloon.

Example 148. The delivery assembly of any example herein, particularly example 141, wherein the hole dilation balloon and the valve expansion balloon are integrally formed, such that the hole dilation balloon is a distal portion of a unitary balloon, and the valve expansion balloon is a proximal portion of the unitary balloon.

Example 149. The delivery assembly of any example herein, particularly example 141, wherein the hole dilation balloon comprises a dilation balloon proximal attachment segment, and the valve expansion balloon comprises an expansion balloon distal attachment segment which is attached to the dilation balloon proximal attachment segment.

Example 150. The delivery assembly of any example herein, particularly any one of examples 141 to 149, wherein the delivery apparatus further comprises a cover shaft disposed around and axially movable relative to the balloon catheter.

Example 151. The delivery assembly of any example herein, particularly example 150, wherein the cover shaft is movable between a first position and a second position, wherein a distal end of the cover shaft is disposed around the valve expansion balloon when the cover shaft is in the first position, and wherein the distal end of the cover shaft is proximal to the valve expansion balloon when the cover shaft is in the second position.

Example 152. The delivery assembly of any example herein, particularly example 151, wherein the distal end of the cover shaft is disposed between the valve expansion balloon and the prosthetic valve in the first position.

Example 153. The delivery assembly of any example herein, particularly example 151, wherein the distal end of the cover shaft is disposed around both the valve expansion balloon and the prosthetic valve in the first position.

Example 154. The delivery assembly of any example herein, particularly any one of examples 141 to 144, wherein the hole dilation balloon comprises a dilation balloon wall having a wall thickness T1, and wherein the valve expansion balloon comprises an expansion balloon wall having a wall thickness T2, and wherein the wall thickness T2 is greater than the wall thickness T1.

Example 155. The delivery assembly of any example herein, particularly example 154, wherein the wall thickness T1 is configured to allow the hole dilation balloon to be inflated when filled by inflation fluid at a pressure less than a pressure threshold, wherein the wall thickness T2 is configured to restrict inflation of the valve expansion balloon when filled by the inflation fluid at a pressure less than a pressure threshold, yet allow the valve expansion balloon to be inflated when filled by the inflation fluid at a pressure greater than or equal to the pressure threshold.

Example 156. The delivery assembly of any example herein, particularly any one of examples 136 to 140, wherein the hole dilation balloon is in fluid communication with the nosecone shaft lumen.

Example 157. The delivery assembly of any example herein, particularly example 156, wherein the valve expansion balloon and the hole dilation balloon are fluidly separated from each other.

Example 158. The delivery assembly of any example herein, particularly example 157, wherein the delivery apparatus further comprises an intermediate seal disposed between the dilation balloon cavity and the expansion balloon cavity.

Example 159. The delivery assembly of any example herein, particularly example 157 or 158, wherein the hole dilation balloon comprises a dilation balloon proximal attachment segment which is attached to the nosecone shaft.

Example 160. The delivery assembly of any example herein, particularly example 159, wherein the delivery apparatus further comprises a distal seal disposed around the nosecone shaft, distally to the dilation balloon cavity.

Example 161. The delivery assembly of any example herein, particularly any one of examples 133 to 160, wherein the hole dilation balloon comprises a distal shoulder, a proximal shoulder, and a dilation balloon main segment extending between the distal shoulder and the proximal shoulder, and wherein the diameters of the proximal and distal shoulders are greater than the diameter of the dilation balloon main segment in the inflated state of the hole dilation balloon.

Example 162. The delivery assembly of any example herein, particularly any one of examples 113 to 132, wherein the first balloon is an inner balloon comprising an inner balloon wall, and wherein the second balloon is an outer balloon disposed around the inner balloon, the outer balloon comprising an outer balloon wall.

Example 163. The delivery assembly of any example herein, particularly example 162, wherein the delivery apparatus further comprises a nosecone shaft extending through the balloon catheter lumen and defining a nosecone shaft lumen.

Example 164. The delivery assembly of any example herein, particularly example 163, wherein the delivery apparatus further comprises a nosecone attached to a distal end of the nosecone shaft.

Example 165. The delivery assembly of any example herein, particularly example 164, wherein the nosecone is distal to the inner balloon.

Example 166. The delivery assembly of any example herein, particularly any one of examples 163 to 165, wherein the perforating member extends through the nosecone shaft.

Example 167. The delivery assembly of any example herein, particularly example 166, wherein the perforating member is axially movable relative to the nosecone shaft.

Example 168. The delivery assembly of any example herein, particularly any one of examples 162 to 167, wherein the inner balloon is mounted on the balloon catheter and is in fluid communication with the balloon catheter lumen.

Example 169. The delivery assembly of any example herein, particularly example 168, wherein the inner balloon is configured to transition from a sealed configuration to an unsealed configuration.

Example 170. The delivery assembly of any example herein, particularly example 169, wherein the inner balloon wall is thinner than the outer balloon wall.

Example 171. The delivery assembly of any example herein, particularly example 170, wherein the thickness of the inner balloon wall is configured to allow the inner balloon to be inflated when filled with inflation fluid at a pressure which is less than a pressure threshold, while the inner balloon is maintained in a sealed configuration, and is configured to tear when the pressure of the inflation fluid exceeds the pressure threshold, thereby transitioning the inner balloon to the unsealed configuration.

Example 172. The delivery assembly of any example herein, particularly example 169, wherein the inner balloon wall comprises a weakened region which is weaker than the outer balloon wall.

Example 173. The delivery assembly of any example herein, particularly example 172, wherein the weakened region is a thinned region which is thinner than the outer balloon wall.

Example 174. The delivery assembly of any example herein, particularly example 173, wherein the thinned region is configured to allow the inner balloon to be inflated when filled with inflation fluid at a pressure which is less than a pressure threshold, while the inner balloon is maintained in a sealed configuration, and is configured to tear when the pressure of the inflation fluid exceeds the pressure threshold, thereby transitioning the inner balloon to the unsealed configuration.

Example 175. The delivery assembly of any example herein, particularly example 171 or 174, wherein the outer balloon wall is configured to withstand an inflation fluid pressure greater than or equal to the pressure threshold without tearing.

Example 176. The delivery assembly of any example herein, particularly example 169, wherein the inner balloon wall comprises a tear-line and the delivery apparatus further comprises a ripcord comprising a ripcord distal end attached to the tear line.

Example 177. The delivery assembly of any example herein, particularly example 176, wherein the tear-line is configured to remain intact as long as the ripcord is not pulled in a proximal direction.

Example 178. The delivery assembly of any example herein, particularly example 176 or 177, wherein the tear-line is configured to tear when the ripcord is pulled in a proximal direction, so as to transition the inner balloon to the unsealed configuration.

Example 179. The delivery assembly of any example herein, particularly any one of examples 176 to 178, wherein the ripcord extends from the ripcord distal end to the handle.

Example 180. The delivery assembly of any example herein, particularly example 169, wherein the inner balloon wall comprises a flexible port which is folded over itself in a sealed configuration of the inner balloon, so as to prevent fluid flow through the flexible port when folded over itself.

Example 181. The delivery assembly of any example herein, particularly example 180, wherein the flexible port is configured to allow fluid flow therethrough when unfolded, so as to transition the inner balloon to the unsealed configuration.

Example 182. The delivery assembly of any example herein, particularly example 181, wherein the delivery apparatus further comprises a loop disposed around the flexible port and configured to retain it folded over itself in a sealed configuration of the inner balloon.

Example 183. The delivery assembly of any example herein, particularly example 182, wherein the delivery apparatus further comprises a release cord attached to the loop, wherein the release cord is configured, when pulled in a proximal direction, to release the loop from the flexible port, allowing the flexible port to unfold and the inner balloon to transition to the unsealed configuration.

Example 184. The delivery assembly of any example herein, particularly example 183, wherein the release cord extends from the loop to the handle.

Example 185. The delivery assembly of any example herein, particularly any one of examples 163 to 167, wherein the outer balloon is mounted on the balloon catheter and is in fluid communication with the balloon catheter lumen.

Example 186. The delivery assembly of any example herein, particularly example 185, wherein the inner balloon is in fluid communication with the nosecone shaft lumen.

Example 187. The delivery assembly of any example herein, particularly example 186, wherein the inner balloon and the outer balloon are fluidly separated from each other.

Example 188. The delivery assembly of any example herein, particularly example 187, wherein the balloon catheter terminates proximal to the inner balloon.

Example 189. The delivery assembly of any example herein, particularly example 187 or 188, wherein the delivery apparatus further comprises a distal seal disposed around the nosecone shaft, distally to the inner balloon.

Example 190. The delivery assembly of any example herein, particularly any one of examples 187 to 189, wherein the inner balloon comprises an inner balloon proximal attachment segment which is attached to the nosecone shaft.

Example 191. The delivery assembly of any example herein, particularly any one of examples 113 to 190, wherein the host valvular structure is a native valvular structure of native heart valve.

Example 192. The delivery assembly of any example herein, particularly any one of examples 113 to 191, wherein the host valvular structure is a valvular structure of previously implanted prosthetic valve that is implanted within a native heart valve.

Example 193. The delivery assembly of any example herein, particularly any one of examples 113 to 192, wherein the delivery apparatus is sterilized.

Example 194. A method of implanting a prosthetic valve within a host valvular structure, the method comprising:
advancing a delivery assembly that comprises a delivery apparatus carrying a prosthetic valve in a radially compressed state, to a host valvular structure, wherein the delivery apparatus comprises a perforating member, a first balloon, and a second balloon;
forming, with the perforating member and the first balloon, a leaflet opening within a host leaflet of the host valvular structure; and
inflating the second balloon, while the prosthetic valve is disposed therearound, inside the host valvular structure, so as to radially expand the prosthetic valve.

Example 195. The method of any example herein, particularly example 194, wherein the forming the leaflet opening comprises forming, with the perforating member, a pilot puncture within the host leaflet.

Example 196. The method of any example herein, particularly example 195, wherein the forming the leaflet opening further comprises positioning the first balloon, in a deflated state thereof, within the pilot puncture.

Example 197. The method of any example herein, particularly example 196, wherein the forming the leaflet opening further comprises inflating the first balloon to expand the pilot puncture and form the leaflet opening within the host leaflet.

Example 198. The method of any example herein, particularly example 197, wherein the first balloon is inflatable to a maximum diameter D1.

Example 199. The method of any example herein, particularly example 196 or 197, further comprising, subsequent to inflating the first balloon and prior to inflating the second balloon, deflating the first balloon.

Example 200. The method of any example herein, particularly example 198, wherein the inflating the second balloon comprises inflating the second balloon to a diameter that exceeds D1.

Example 201. The method of any example herein, particularly any one of examples 194 to 200, further comprising, prior to the inflating the second balloon, positioning the second balloon in a deflated state thereof, with the prosthetic valve disposed in a compressed state over the second balloon, inside the host valvular structure.

Example 202. The method of any example herein, particularly example 201, wherein the positioning the second balloon inside the host valvular structure comprises positioning the second balloon between host leaflets of the valvular structure.

Example 203. The method of any example herein, particularly example 202, further comprising, prior to positioning the second balloon, proximally retracting the deflated first balloon out of the leaflet opening.

Example 204. The method of any example herein, particularly any one of examples 194 to 203, wherein the inflating the second balloon inside the host valvular structure comprises inflating the second balloon between host leaflets of the valvular structure.

Example 205. The method of any example herein, particularly example 201, wherein the positioning the second balloon inside the host valvular structure comprises positioning the second balloon inside the leaflet opening.

Example 206. The method of any example herein, particularly any one of examples 194 to 201, wherein the inflating the second balloon inside the host valvular structure comprises inflating the second balloon inside the leaflet opening.

Example 207. The method of any example herein, particularly example 205 or 206, wherein the inflating the second balloon to radially expand the prosthetic valve increases the size of the leaflet opening.

Example 208. The method of any example herein, particularly any one of examples 205 to 207, wherein the inflating the second balloon to radially expand the prosthetic valve tears the host leaflet.

Example 209. The method of any example herein, particularly any one of examples 194 to 208, wherein the inflating the second balloon to radially expand the prosthetic valve modifies the host leaflet such that the host leaflet does not obstruct a cell opening of a frame of the prosthetic valve.

Example 210. The method of any example herein, particularly any one of examples 194 to 208, wherein the inflating the second balloon to radially expand the prosthetic valve moves the host leaflet to a location upstream of a downstream edge of an outer skirt of the prosthetic valve.

Example 211. The method of any example herein, particularly any one of examples 194 to 210, further comprising, prior to forming the leaflet opening, positioning the perforating member adjacent the host leaflet.

Example 212. The method of any example herein, particularly any one of examples 195 to 200, wherein the forming the pilot puncture comprises translating the perforating member in a distal direction relative to the first balloon to pierce the host leaflet to form the pilot puncture.

Example 213. The method of any example herein, particularly any one of examples 195 to 200, wherein the perforating member comprises a needle.

Example 214. The method of any example herein, particularly any one of examples 195 to 200, wherein the perforating member comprises a perforating member lumen.

Example 215. The method of any example herein, particularly example 214, wherein the forming the pilot puncture comprises perforating the host leaflet by a guidewire extending through the perforating member, followed by piercing the host leaflet by the perforation member, advanced over the guidewire to form the pilot puncture.

Example 216. The method of any example herein, particularly example 215, wherein the perforating the host leaflet by the guidewire comprises applying RF energy to a tip of the guidewire.

Example 217. The method of any example herein, particularly any one of examples 195 to 201, wherein the perforating member is a guidewire.

Example 218. The method of any example herein, particularly example 217, wherein the forming the pilot puncture comprises applying RF energy to a tip of the guidewire.

Example 219. The method of any example herein, particularly any one of examples 194 to 218, further comprising, prior to inflating the second balloon inside the host valvular structure, distally pushing the prosthetic valve, by a push shaft of the delivery apparatus, towards and over the second balloon.

Example 220. The method of any example herein, particularly example 219, further comprising, subsequent to distally pushing the prosthetic valve, keeping the push shaft in close proximity to a proximal end of the prosthetic valve, so as to provide a counterforce to prevent the prosthetic valve from proximally slipping from the second balloon.

Example 221. The method of any example herein, particularly any one of examples 196 to 200, wherein the delivery apparatus further comprises a nosecone distal to the first balloon, and a nosecone shaft attached to the nosecone.

Example 222. The method of any example herein, particularly example 221, wherein the positioning the first balloon within the pilot puncture comprises advancing the nosecone through the pilot puncture.

Example 223. The method of any example herein, particularly example 221 or 222, wherein the perforating member extends through a lumen of the nosecone shaft.

Example 224. The method of any example herein, particularly example 223, wherein the forming the pilot puncture comprises translating the perforating member in a distal direction relative to the nosecone to pierce the host leaflet to form the pilot puncture.

Example 225. The method of any example herein, particularly example 223 or 224, further comprising, subsequent to forming the pilot puncture and prior to inflating the second balloon, retracting the perforating member into the nosecone shaft.

Example 226. The method of any example herein, particularly any one of examples 194 to 220, wherein the first balloon is a hole dilation balloon, and wherein the second balloon is a valve expansion balloon proximal to the hole dilation balloon.

Example 227. The method of any example herein, particularly example 226, wherein the delivery apparatus further comprises a balloon catheter defining a balloon catheter lumen, and wherein the valve expansion balloon is in fluid communication with the balloon catheter lumen.

Example 228. The method of any example herein, particularly example 226 or 227, wherein the hole dilation balloon is in fluid communication with the valve expansion balloon.

Example 229. The method of any example herein, particularly example 228, wherein the delivery apparatus further comprises an intermediate connector disposed between an internal cavity of the valve expansion balloon and an internal cavity of the hole dilation balloon, wherein the intermediate connector comprises an intermediate channel fluidly connecting between the cavities of the valve expansion balloon and the hole dilation balloon.

Example 230. The method of any example herein, particularly example 228 or 229, wherein the hole dilation balloon and the valve expansion balloon are integrally formed, such that the hole dilation balloon is a distal portion of a unitary balloon, and the valve expansion balloon is a proximal portion of the unitary balloon.

Example 231. The method of any example herein, particularly example 228 or 229, wherein the hole dilation balloon comprises a dilation balloon proximal attachment segment, and the valve expansion balloon comprises an expansion balloon distal attachment segment which is attached to the dilation balloon proximal attachment segment.

Example 232. The method of any example herein, particularly any one of examples 228 to 231, wherein the inflating the first balloon comprises keeping a cover shaft of the delivery apparatus disposed around the valve expansion balloon, wherein the cover shaft is configured to restrict expansion of the valve expansion balloon during inflation of the hole dilation balloon.

Example 233. The method of any example herein, particularly example 232, further comprising, prior to inflating the second balloon, retracting the cover shaft proximally to the valve expansion balloon.

Example 234. The method of any example herein, particularly example 233, wherein the cover shaft is disposed between the prosthetic valve and the valve expansion balloon prior to retracting the cover shaft.

Example 235. The method of any example herein, particularly example 233, wherein the cover shaft is disposed around the prosthetic valve and the valve expansion balloon prior to retracting the cover shaft.

Example 236. The method of any example herein, particularly any one of examples 228 to 231, wherein the valve expansion balloon comprises an expansion balloon wall having a wall thickness T2 which is greater than a wall thickness T1 of a dilation balloon wall of the hole dilation balloon.

Example 237. The method of any example herein, particularly example 236, wherein the forming the leaflet opening comprises inflating the hole dilation balloon by providing inflation fluid at a pressure which is lower than a pressure threshold, sufficient to inflate the hole inflation balloon, yet lower than the pressure threshold required to inflate the thicker valve expansion balloon.

Example 238. The method of any example herein, particularly example 237, wherein the inflating the valve expansion balloon comprises providing inflation fluid at a pressure exceeding the pressure threshold.

Example 239. The method of any example herein, particularly any one of examples 228 to 231, wherein the forming the leaflet opening comprises inflating the hole dilation balloon by providing inflation fluid at a volume which is sufficient to simultaneously inflate both the hole dilation balloon and the valve expansion balloon, without the valve expansion balloon expanding to a diameter greater than the diameter of the hole dilation balloon.

Example 240. The method of any example herein, particularly example 239, wherein the inflating the valve expansion balloon comprises providing inflation fluid at a volume which is sufficient to inflate the valve expansion balloon to a diameter greater than that of the hole dilation balloon.

Example 241. The method of any example herein, particularly example 226, wherein the valve expansion balloon and the hole dilation balloon are fluidly separated.

Example 242. The method of any example herein, particularly example 241, wherein the delivery apparatus further comprises a balloon catheter defining a balloon catheter lumen, wherein the valve expansion balloon is in fluid communication with the balloon catheter lumen, and wherein the hole expansion balloon is in fluid communication with a lumen of a nosecone shaft of the delivery apparatus.

Example 243. The method of any example herein, particularly example 242, wherein the delivery apparatus further comprises an intermediate seal, sealing between an internal cavity of the hole dilation balloon and an internal cavity of the valve expansion balloon.

Example 244. The method of any example herein, particularly example 242 or 243, wherein the delivery apparatus further comprises a distal seal disposed around the nosecone shaft, positioned distally to the hole dilation balloon.

Example 245. The method of any example herein, particularly any one of examples 242 to 244, wherein the hole dilation balloon comprises a dilation balloon proximal attachment segment which is attached to a nosecone shaft of the delivery apparatus.

Example 246. The method of any example herein, particularly any one of examples 242 to 245, wherein the forming the leaflet opening comprises inflating the hole dilation balloon by providing inflation fluid into the hole dilation balloon via a lumen of the nosecone shaft.

Example 247. The method of any example herein, particularly any one of examples 242 to 246, wherein the inflating the valve expansion balloon comprises providing inflation fluid into the valve expansion balloon via the balloon catheter lumen.

Example 248. The method of any example herein, particularly any one of examples 194 to 220, wherein the first balloon is an inner balloon, and wherein the second balloon is an outer balloon disposed around the inner balloon.

Example 249. The method of any example herein, particularly example 248, wherein, during the advancing the delivery assembly to the host valvular structure, the inner balloon is in a sealed configuration, in which it is fluidly sealed from a cavity of the outer balloon.

Example 250. The method of any example herein, particularly example 249, wherein the delivery apparatus further comprises a balloon catheter defining a balloon catheter lumen, and wherein the inner balloon is in fluid communication with the balloon catheter lumen.

Example 251. The method of any example herein, particularly example 250, wherein the inner balloon comprises an inner balloon wall, wherein the outer balloon comprises an outer balloon wall, and wherein the method further comprises, subsequent to forming the leaflet opening and prior to inflating the outer balloon, transitioning the inner balloon to an unsealed configuration.

Example 252. The method of any example herein, particularly example 251, wherein the inner balloon wall is thinner than the outer balloon wall.

Example 253. The method of any example herein, particularly example 252, wherein the forming the leaflet opening comprises inflating the inner balloon by providing inflation fluid to the inner balloon at a pressure that does not exceed a pressure threshold, so as to inflate the inner balloon while maintaining the inner balloon in the sealed configuration without tearing the inner balloon wall.

Example 254. The method of any example herein, particularly example 253, wherein the transitioning the inner balloon to the unsealed configuration comprises providing inflation fluid to the inner balloon at a pressure that is greater than or equal to the pressure threshold, thereby tearing the inner balloon wall.

Example 255. The method of any example herein, particularly example 251, wherein the inner balloon wall comprises a weakened region which is weaker than the outer balloon wall.

Example 256. The method of any example herein, particularly example 255, wherein the weakened region is a thinned region which is thinner than the outer balloon wall.

Example 257. The method of any example herein, particularly example 256, wherein the forming the leaflet opening comprises inflating the inner balloon by providing inflation fluid to the inner balloon at a pressure that does not exceed a pressure threshold, so as to inflate the inner balloon while maintaining the inner balloon in a sealed configuration without tearing the thinned region.

Example 258. The method of any example herein, particularly example 256 or 257, wherein the transitioning the inner balloon to the unsealed configuration comprises providing inflation fluid to the inner balloon at a pressure that is greater than or equal to the pressure threshold, thereby tearing the weakened region.

Example 259. The method of any example herein, particularly any one of examples 252 to 258, wherein the outer balloon wall is configured to withstand an inflation fluid pressure that is greater than or equal to the pressure threshold without tearing.

Example 260. The method of any example herein, particularly example 251, wherein the inner balloon wall comprises a tear-line and the delivery apparatus further comprises a ripcord comprising a ripcord distal end attached to the tear line.

Example 261. The method of any example herein, particularly example 260, wherein the tear-line is configured to remain intact during the inflating the inner balloon.

Example 262. The method of any example herein, particularly example 260 or 261, wherein the transitioning the inner balloon to an unsealed configuration comprises pulling the ripcord in a proximal direction, thereby tearing the tear-line.

Example 263. The method of any example herein, particularly example 251, wherein the inner balloon comprises a flexible port extending sideways from the inner balloon wall, and wherein the inflating the inner balloon comprises maintaining the flexible port folded over itself in a manner that prevent fluid flow therethrough.

Example 264. The method of any example herein, particularly example 263, wherein the delivery apparatus further comprises a loop disposed around the flexible port when folded over itself, and wherein maintaining the flexible port folded over itself comprises maintaining the loop disposed around a port fold of the flexible port.

Example 265. The method of any example herein, particularly example 264, wherein the delivery apparatus further comprises a release cord attached to the loop.

Example 266. The method of any example herein, particularly example 265, wherein the transitioning the inner balloon to the unsealed configuration comprises pulling the release cord in a proximal direction, thereby releasing the loop from the flexible port and allowing the flexible port to unfold and allow fluid flow therethrough.

Example 267. The method of any example herein, particularly example 249, wherein the delivery apparatus further comprises a nosecone shaft defining a nosecone shaft lumen, and wherein the inner balloon is in fluid communication with the nosecone shaft lumen.

Example 268. The method of any example herein, particularly example 267, wherein the delivery apparatus further comprises a balloon catheter defining a balloon catheter lumen, and wherein the outer balloon is in fluid communication with the balloon catheter lumen.

Example 269. The method of any example herein, particularly example 268, wherein the forming the leaflet opening comprises inflating the inner balloon by providing inflation fluid into the inner balloon via the nosecone shaft lumen.

Example 270. The method of any example herein, particularly example 268 or 269, wherein the inflating the outer balloon comprises providing inflation fluid into the outer balloon via the balloon catheter lumen.

Example 271. The method of any example herein, particularly any one of examples 194 to 270, wherein the host valvular structure is a native valvular structure of a native heart valve.

Example 272. The method of any example herein, particularly any one of examples 194 to 270, wherein the host valvular structure is a valvular structure of previously implanted prosthetic valve that is implanted within a native heart valve.

Example 273. The method of any example herein, particularly any one of examples 194 to 272, wherein the forming the leaflet opening within the host leaflet comprises forming a first leaflet opening within a first host leaflet, and wherein, subsequent to forming the first leaflet opening, the method further comprises:
retracting the delivery apparatus from the first host leaflet; and
forming, with the perforating member and the first balloon, a second leaflet opening within a second host leaflet of the host valvular structure.

Example 274. The method of any example herein, particularly example 273, wherein the inflating the second inside the host valvular structure comprises inflating the second balloon inside the second leaflet opening.

Example 275. The method of any example herein, particularly any one of examples 194 to 274, further comprising, subsequent to inflating the valve expansion balloon to radially expand the prosthetic valve, deflating the valve expansion balloon and retrieving the delivery apparatus.

Example 276. A delivery assembly, comprising:
a guest prosthetic valve comprising a frame movable between a radially compressed and a radially expanded configuration; and
a delivery apparatus comprising:
   a handle;
   a balloon catheter extending from the handle, the balloon catheter defining a balloon catheter lumen;
   a valve expansion balloon mounted on the balloon catheter and in fluid communication with the balloon catheter lumen, the valve expansion balloon configured to transition between deflated and inflated states thereof;
   a hole dilation balloon positioned distal to the valve expansion balloon and in fluid communication with the valve expansion balloon, the hole dilation balloon configured to transition between deflated and inflated states thereof; and
   a perforating member configured to pierce a host leaflet of a host valvular structure to form a pilot puncture in the host leaflet, when a distal end portion of the perforating member is positioned distal to the hole dilation balloon;
wherein the maximum diameter of the valve expansion balloon in its inflated state is greater than the maximum diameter of the hole dilation balloon in its inflated state;
wherein the hole dilation balloon is configured to be inserted within the pilot puncture;
wherein inflation of the hole dilation balloon, when positioned within the pilot puncture, is configured to expand the pilot puncture to form a leaflet opening; and
wherein, when the guest prosthetic valve is disposed around the valve expansion balloon and positioned within the host valvular structure, inflation of the valve expansion balloon expands the guest prosthetic valve to implant the guest prosthetic valve in the host valvular structure.

Example 277. A delivery assembly, comprising:
a guest prosthetic valve comprising a frame movable between a radially compressed and a radially expanded configuration; and
a delivery apparatus comprising:
   a handle;
   a balloon catheter extending from the handle and defining a balloon catheter lumen;
   a nosecone shaft extending through the balloon catheter lumen and defining a nosecone shaft lumen;
   a nosecone attached to a distal end of the nosecone shaft;
   a valve expansion balloon mounted on the balloon catheter and in fluid communication with the balloon catheter lumen, the valve expansion balloon configured to transition between deflated and inflated states thereof;
   a hole dilation balloon positioned distal to the valve expansion balloon and in fluid communication with the nosecone shaft lumen, the hole dilation balloon configured to transition between deflated and inflated states thereof; and
   a perforating member configured to pierce a host leaflet of a host valvular structure to form a pilot puncture in the host leaflet, when a distal end portion of the perforating member is positioned distal to the hole dilation balloon;
wherein the maximum diameter of the valve expansion balloon in its inflated state is greater than the maximum diameter of the hole dilation balloon in its inflated state;
wherein the valve expansion balloon and the hole dilation balloon are fluidly separated from each other;
wherein the hole dilation balloon is configured to be inserted within the pilot puncture;
wherein inflation of the hole dilation balloon, when positioned within the pilot puncture, is configured to expand the pilot puncture to form a leaflet opening; and
wherein, when the guest prosthetic valve is disposed around the valve expansion balloon and positioned within the host valvular structure, inflation of the valve expansion balloon expands the guest prosthetic valve to implant the guest prosthetic valve in the host valvular structure.

Example 278. A delivery assembly, comprising:
a guest prosthetic valve comprising a frame movable between a radially compressed and a radially expanded configuration; and
a delivery apparatus comprising:
   a handle;
   a balloon catheter extending from the handle and defining a balloon catheter lumen;
   an inner balloon mounted on the balloon catheter and in fluid communication with the balloon catheter lumen, the inner balloon comprising an inner balloon wall, wherein the inner balloon is configured to transition between deflated and inflated states thereof;
   an outer balloon disposed around the inner balloon, the outer balloon comprising an outer balloon wall, wherein the outer balloon is configured to transition between deflated and inflated states thereof; and
   a perforating member configured to pierce a host leaflet of a host valvular structure to form a pilot puncture in the host leaflet, when a distal end portion of the perforating member is positioned distal to the inner;
wherein the maximum diameter of the outer balloon in its inflated state is greater than the maximum diameter of the inner balloon in its inflated state;
wherein the inner balloon is configured to transition from a sealed configuration to an unsealed configuration;
wherein the inner and outer balloons are configured to be inserted within the pilot puncture;
wherein inflation of the inner balloon, when the inner and outer balloons are positioned within the pilot puncture, is configured to expand the pilot puncture to form a leaflet opening; and
wherein, when the guest prosthetic valve is disposed around the outer balloon and positioned within the host valvular structure, inflation of the outer balloon expands the guest prosthetic valve to implant the guest prosthetic valve in the host valvular structure.

Example 279. A delivery assembly, comprising:
a guest prosthetic valve comprising a frame movable between a radially compressed and a radially expanded configuration; and
a delivery apparatus comprising:
   a handle;
   a balloon catheter extending from the handle and defining a balloon catheter lumen;
   a nosecone shaft extending through the balloon catheter lumen and defining a nosecone shaft lumen;
   a nosecone attached to a distal end of the nosecone shaft;
   an outer balloon mounted on the balloon catheter and in fluid communication with the balloon catheter lumen, the outer balloon configured to transition between deflated and inflated states thereof;
   an inner balloon disposed inside the outer balloon and in fluid communication with the nosecone shaft lumen, the inner balloon configured to transition between deflated and inflated states thereof; and
   a perforating member configured to pierce a host leaflet of a host valvular structure to form a pilot puncture in the host leaflet, when a distal end portion of the perforating member is positioned distal to the inner balloon;
wherein the maximum diameter of the outer balloon in its inflated state is greater than the maximum diameter of the inner balloon in its inflated state;
wherein the inner balloon and the outer balloon are fluidly separated from each other;
wherein the inner and outer balloons are configured to be inserted within the pilot puncture;
wherein inflation of the inner balloon via the nosecone shaft lumen, when the inner and outer balloons are positioned within the pilot puncture, is configured to expand the pilot puncture to form a leaflet opening; and
wherein, when the guest prosthetic valve is disposed around the outer balloon and positioned within the host valvular structure, inflation of the outer balloon via the balloon catheter lumen expands the guest prosthetic valve to implant the guest prosthetic valve in the host valvular structure.

Example 280. A method of implanting a guest prosthetic valve within a host valvular structure, the method comprising:
advancing a delivery assembly that comprises a delivery apparatus carrying a guest prosthetic valve in a radially compressed state, to a host valvular structure, wherein the delivery apparatus comprises a perforating member, a hole dilation balloon, and a valve expansion balloon proximal to the hole dilation balloon,
forming, with the perforating member, a pilot puncture within a host leaflet of the host valvular structure;
positioning the hole dilation balloon, in a deflated state thereof, within the pilot puncture;
inflating the hole dilation balloon to expand the pilot puncture and form a leaflet opening within the host leaflet, wherein the hole dilation balloon is inflatable to a maximum diameter D1;
deflating the hole dilation balloon;
positioning the valve expansion balloon in a deflated state thereof, along with the guest prosthetic valve disposed in a compressed state over the valve expansion balloon, inside the host valvular structure; and
inflating the valve expansion balloon to a diameter that exceeds D1, so as to radially expand the guest prosthetic valve.

Example 281. A method of implanting a guest prosthetic valve within a host valvular structure, the method comprising:
advancing a delivery assembly that comprises a delivery apparatus carrying a guest prosthetic valve in a radially compressed state, to a host valvular structure, wherein the delivery apparatus comprises an inner balloon, an outer balloon disposed around the inner balloon, and a perforating member, wherein the inner balloon is in a sealed configuration, in which it is fluidly sealed from a cavity of the outer balloon, during advancing of the delivery assembly to the host valvular structure;
forming, with the perforating member, a pilot puncture within a host leaflet of the host valvular structure;
positioning the inner and outer balloons, in deflated states thereof, within the pilot puncture;
inflating the inner balloon to expand the pilot puncture and form a leaflet opening within the host leaflet, wherein the inner balloon is inflatable to a maximum diameter D1; and
inflating the outer balloon with the guest prosthetic valve disposed in a compressed state thereover, inside the host valvular structure to a diameter that exceeds D1, so as to radially expand the guest prosthetic valve.

It is appreciated that certain features of the invention, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

We claim:

1. A leaflet perforation tool, comprising:
a catheter comprising a distal end portion configured to be inserted into a patient's vasculature, wherein the catheter comprises a lumen;
a lacerating member disposed within the lumen of the catheter, wherein the lacerating member is configured to pierce a leaflet of a host valvular structure to form a pilot puncture in the leaflet; and
an expansion member supported by the distal end portion of the catheter, wherein the expansion member is configured to be inserted within the pilot puncture and to be selectively transitioned between a radially compressed configuration and a radially expanded configuration;
wherein, when the expansion member is received within the pilot puncture, transitioning the expansion member from the radially compressed configuration to the radially expanded configuration expands the pilot puncture to form a leaflet opening.

2. The leaflet perforation tool of claim 1, wherein the lacerating member comprises a distal end portion that is configured to be selectively translated in a proximal direction and in a distal direction relative to the distal end portion of the catheter.

3. The leaflet perforation tool of claim 1, wherein the lacerating member comprises a needle.

4. The leaflet perforation tool of claim 1, wherein the lacerating member comprises a lumen that is configured to receive a perforation tool guidewire of the leaflet perforation tool.

5. The leaflet perforation tool of claim 1, wherein the lacerating member comprises a guidewire.

6. The leaflet perforation tool of claim 4, further comprising an RF energy source coupled to the guidewire and configured to provide RF energy to a tip of the guidewire.

7. The leaflet perforation tool of claim 1, wherein the expansion member extends at least substantially around a circumference of the catheter.

8. The leaflet perforation tool of claim 1, wherein the expansion member comprises an inflatable balloon that is configured to be selectively inflated and deflated to transition the expansion member between the radially compressed configuration and the radially expanded configuration.

9. The leaflet perforation tool of claim 1, wherein the expansion member comprises an expandable frame comprising a collapsible support structure that is radially expandable and compressible to transition the expandable frame between the radially compressed configuration and the radially expanded configuration.

10. The leaflet perforation tool of claim 9, wherein the collapsible support structure comprises a plurality of frame members arranged and connected such that the collapsible support structure expands radially as the collapsible support structure is compressed axially.

11. The leaflet perforation tool of claim 9, wherein the expandable frame comprises a distal end portion and a proximal end portion that are operatively coupled to one another via a screw mechanism, wherein rotation of the screw mechanism in a first direction causes a distance between the distal end portion and the proximal end portion to decrease and produce radial expansion of the expandable frame, and wherein rotation of the screw mechanism is a second direction causes a distance between the distal end portion and the proximal end portion to increase and produce radial compression of the expandable frame.

12. The leaflet perforation tool of claim 1, further comprising one or more positioning arms supported by the catheter and configured to facilitate positioning the catheter relative to the host valvular structure.

13. A method of implanting a replacement prosthetic valve within a host valvular structure, the method comprising:
forming, with a leaflet perforation tool, a leaflet opening within a leaflet of the host valvular structure;
positioning a replacement prosthetic valve in a radially compressed configuration within the leaflet opening; and
radially expanding the replacement prosthetic valve.

14. The method of claim 13, wherein the forming the leaflet opening comprises:
forming, with the leaflet perforation tool, a pilot puncture within the leaflet;
inserting a portion of the leaflet perforation tool into the pilot puncture; and
expanding the pilot puncture to form the leaflet opening.

15. The method of claim 14, wherein the leaflet perforation tool comprises a catheter and a lacerating member disposed within a lumen of the catheter, and wherein the forming the pilot puncture within the leaflet comprises translating the lacerating member in a distal direction relative to the catheter to pierce the leaflet to form the pilot puncture.

16. The method of claim 13, wherein the leaflet perforation tool comprises a catheter and an expansion member supported by the catheter, and wherein the forming the leaflet opening comprises:
forming, with the leaflet perforation tool, a pilot puncture within the leaflet;
positioning the expansion member within the pilot puncture; and
transitioning the expansion member from a radially compressed configuration to a radially expanded configuration to expand the pilot puncture into the leaflet opening.

17. The method of claim 16, wherein the leaflet perforation tool comprises a lacerating member disposed within a lumen of the catheter, wherein the forming the pilot puncture within the leaflet comprises translating the lacerating member in a distal direction relative to the catheter to pierce the leaflet to form the pilot puncture, and where the method further comprises, subsequent to the forming the pilot puncture and prior to the transitioning the expansion member from the radially compressed configuration to the radially expanded configuration, retracting the lacerating member away from the leaflet.

18. The method of claim 16, wherein the expansion member comprises an inflatable balloon, and wherein the transitioning the expansion member from the radially compressed configuration to the radially expanded configuration comprises inflating the inflatable balloon to transition the inflatable balloon from a deflated state to an inflated state.

19. The method of claim 13, wherein the positioning the replacement prosthetic valve within the leaflet opening comprises advancing the replacement prosthetic valve into the leaflet opening via a replacement valve guidewire.

20. The method of claim 19, wherein the positioning the leaflet perforation tool relative to the leaflet comprises advancing the leaflet perforation tool toward the leaflet via the replacement valve guidewire.

21. The method of claim 19, wherein the positioning the leaflet perforation tool relative to the leaflet comprises advancing the leaflet perforation tool toward the leaflet via a perforation tool guidewire that extends alongside the replacement valve guidewire.

22. The method of claim 13, wherein the radially expanding the replacement prosthetic valve comprises inflating a valve delivery inflatable balloon.

23. The method of claim 13, wherein the leaflet is a first leaflet, wherein the leaflet opening is a first leaflet opening formed in the first leaflet, and wherein the method further comprises:
forming a second leaflet opening within a second leaflet of the host valvular structure.
